(19) United States
(12) Patent Application Publication
(10) Pub. No.: US 2024/0109947 A1
(43) Pub. Date: Apr. 4, 2024

(54) IMMUNOSTIMULATORY CYTOKINE COMBINATION AND THERAPEUTIC USE THEREOF

(71) Applicant: THE TRUSTEES OF DARTMOUTH COLLEGE, Hanover, NH (US)

(72) Inventors: Yina HUANG, Lebanon, NH (US); Rachel BROG, Lebanon, NH (US); Prathna KUMAR, Lebanon, NH (US); Jia ZOU, Lebanon, NH (US)

(21) Appl. No.: 18/359,373

(22) Filed: Jul. 26, 2023

Related U.S. Application Data
(63) Continuation of application No. PCT/US2022/014017, filed on Jan. 27, 2022.
(60) Provisional application No. 63/142,158, filed on Jan. 27, 2021.
(60) Provisional application No. 63/219,620, filed on Jul. 8, 2021.

(51) Int. Cl. C07K 14/55; A61K 39/00; A61P 35/00; C07K 14/54

(52) U.S. Cl. CPC C07K 14/55; A61K 39/4631; A61P 35/00; C07K 14/54; A61K 2239/13; A61K 2239/21

(57) ABSTRACT

Novel cytokine combinations comprising the combination of a type I cytokine, e.g., interleukin-2 (IL-2), or an IL-2 superkine variant (super-2); and a type II cytokine, e.g., IL-33 or IL-25 are provided for use in treating cancer or infection, e.g., solid tumors. These cytokines may be administered in soluble form or may be expressed by cells, e.g., immune cells which express an endogenous or chimeric receptor which binds to target cells, e.g., cancer cells such as those in a solid tumor.

### RELATED APPLICATIONS

The present application claims priority to Provisional Application No. 63/142,158 filed on Jan. 27, 2021, and Provisional Application No. 63/219,620 filed on Jul. 8, 2021, the contents of both of which are incorporated by reference in their entirety herein.

### FIELD OF THE INVENTION

The present invention relates to improved methods for treating cancer or infection, particularly solid tumors using specific cytokine combinations. Also, the invention relates to compositions for use in such methods, e.g., cytokine containing compositions and/or cells which express such cytokines, e.g., CAR-T cells and CAR-T constructs.

### BACKGROUND OF THE INVENTION

Immunotherapy and CAR-T cells in particular have been successfully used for treating many non-solid cancers (e.g., hematological cancers such as leukemias and lymphomas). By contrast the use of immunotherapy and CAR-T cells to treat solid cancers has not been equally successful in part because of the difficulty in delivering the active therapeutic agent, e.g., an antibody, cytolytic enzyme or cytokine to the microenvironment of the solid tumor.

The present invention seeks to address such problems by providing novel methods and compositions that effectively treat solid tumors.

### SUMMARY OF THE INVENTION

The present invention in general relates to an immunostimulatory cytokine combination comprising a type 1 cytokine preferably comprising an IL-21 or IL-2 like cytokine, e.g., human IL-2 or Super-2, and a type 2 cytokine, e.g. IL-33, IL-25 or TSLP et al., compositions which contain and/or recombinant cells engineered to express same and the use of the foregoing as a therapeutic, e.g., to treat cancer or an infectious condition.

In some exemplary embodiments the invention provides a method of treating cancer or infection, the method comprising administering to a subject in need thereof an immunostimulatory cytokine combination comprising a prophylactically therapeutically effective amount of
(a) at least one Type I cytokine which comprises interleukin-21 or an interleukin-2-like (IL-2-like) cytokine, and
(b) at least one Type II cytokine, optionally IL-33 or IL-25, wherein said at least one cytokine (a) and (b) are administered in the same or different compositions, and preferably at dosages wherein said at least one cytokine (a) and (b) in combination elicit a synergistic or additive effect on immunity compared to cytokine (a) or cytokine (b) alone.

In some exemplary embodiments the administered Type 1 cytokine is selected from human IL-2 (IL2), desleukin (Proleukin), Interking (recombinant IL-2 with a serine at residue 125), Neoleukin 2/15, IL2 fused to serum albumin (e.g., MSA/IL-2), or Super-2 (Super2) and other IL2 variants and the Type 2 cytokine is selected from TSLP, IL-25 (IL25), IL-33, IL-1(IL1), IL-4 (IL4), IL-5 (IL5), IL-6 (IL6), IL-10 (IL10)4 (IL4), and IL-13 (IL13).

In some exemplary embodiments the administered immunostimulatory combination comprises Superkine IL-2 (Super-2) and IL-33 or IL-25.

In some exemplary embodiments the administered immunostimulatory combination is used to treat at least one cancer.

In some exemplary embodiments the administered immunostimulatory combination is used to treat at least one solid tumor, optionally wherein the solid tumor is selected from bladder cancer, bone cancer, brain cancer, breast cancer, carcinomas, cervical cancer, colon cancer, colorectal cancer, desmoid tumor, endometrial cancer, esophageal cancer, fibromatosis, glioblastoma, head and neck cancer, liver cancer, lung cancer, lymphoma, leukemias, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, testicular cancer, thyroid cancer, urothelial cancer, or a combination of any of the foregoing.

In some exemplary embodiments the administered immunostimulatory combination additively or synergistically activates the endogenous immune response providing for enhanced inhibition of tumor growth, migration and/or metastasis.

In some exemplary embodiments either or both of said cytokines are administered in soluble form, optionally modified to enhance (increase) in vivo half-life and/or delivery to target cells.

In some exemplary embodiments either or both of said administered cytokines are expressed by a recombinant cell, e.g., an immune cell.

In some exemplary embodiments either or both of said administered cytokines are expressed by a recombinant cell, e.g., a cell which expresses an endogenous or chimeric receptor or binding domain, e.g., an antibody binding domain, which binds to a ligand or receptor expressed by target cells, e.g., cancer or infected cells.

In some exemplary embodiments either or both of said administered cytokines are expressed by a CAR expressing cell, e.g., a CAR-T or CAR-NK cell or other immune cell.

In some exemplary embodiments either or both of said administered cytokines are expressed by cells which express either or both of Super-2 and IL-33 or IL-25 and further express a receptor or binding domain that binds to an antigen or ligand expressed on target cells, e.g., a natural or synthetic or chimeric receptor or an antibody or antibody fragment that binds to target (e.g., tumor) cells.

In some exemplary embodiments the Type I and Type II cytokines are administered separately.

In some exemplary embodiments said at least one Type I and Type II cytokines are administered in combination.

In some exemplary embodiments either or both of the Type I and Type II cytokines are administered as a soluble protein optionally modified to enhance (increase) in vivo half-life and/or delivery to target cells, e.g., via injection, optionally by intraperitoneal administration, intravenous infusion, intramuscularly, intratumorally, subcutaneously; sublingually, intranasally, or other conventional route of administration of soluble proteins.

In some exemplary embodiments said at least one Type I and Type II cytokine in combination elicit a synergistic effect on immunity, optionally on antitumor immunity.

In some exemplary embodiments either or both of said Type I and Type II cytokines are expressed by a recombinant or isolated cell which expresses either or both of said Type I and Type 11 cytokines on the same or different constructs under constitutive or inducible conditions.

In some exemplary embodiments said recombinant or isolated cell optionally comprises an immune cell, further optionally selected from the group consisting of a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH17 cell, a TH9 cell, a TH22 cell, a follicular helper T cells, an a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, an interferon gamma-deficient cell, a B cell, a myeloid cell, a monocyte, a macrophage, an interferon gamma-deficient cell, and a dendritic cell, preferably a CAR-T cell.

In some exemplary embodiments the recombinant or isolated cell expresses at least one natural or chimeric antigen receptor (CAR) which binds to an antigen expressed by target cells, optionally tumor, infected or other diseased cells.

In some exemplary embodiments the recombinant cell expresses at least one chimeric antigen receptor (CAR) comprises:
(a) an antigen-binding (AB) domain that binds to a target molecule which is expressed on the surface of a tumor, a virus, or virus infected cell in a patient or which is overexpressed or aberrantly expressed in cancer, viral infection, or other disease associated with expression of the antigen bound by the AB domain,
(b) a transmembrane (TM) domain,
(c) an intracellular signaling (ICS) domain,
(d) optionally a hinge that joins said AB domain and said TM domain, and
(e) optionally one or more additional costimulatory (CS) domains.

In some exemplary embodiments the administered recombinant or isolated cell comprises at least one nucleic acid encoding a CAR and at least one nucleic acid encoding said Type I cytokine and/or said Type II cytokine as afore described which can be on the same or different nucleic acid constructs.

In some exemplary embodiments:
(i) the cell is activated or stimulated to proliferate when the CAR binds to its target molecule;
(ii) the cell expresses said type 1 and/or said type II 2 cytokine constitutively, or is activated or stimulated to release or secrete said type 1 and/or said type II 2 cytokine when the CAR binds to its target molecule;
(iii) the cell exhibits cytotoxicity against cells expressing the target molecule when the CAR binds to the target molecule;
(iv) administration of the cell alone or in combination with a type I and/or type II cytokine shifts the disease microenvironment from immune suppressive to immune stimulatory;
(v) administration of the cell alone or in combination with a type I and/or type II cytokine increases populations of infiltrating immune cells within the disease microenvironment;
(vi) administration of the cell ameliorates a disease, e.g., an infectious disease or a neoplastic disease, e.g., a solid tumor associated condition, when the CAR binds to its target molecule; and/or
(vii) administration of the cell alone or in combination with a type I or type II cytokine results in enhanced killing of target cells because of the additive or synergistic effects of the combination of the type I and type II cytokine on immunity, optionally antitumor immunity.

In some exemplary embodiments:
(a) both Type I and Type II cytokines are administered as soluble proteins,
(b) both Type I and Type II cytokines are expressed by a recombinant or isolated cell, e.g., a CAR-T cell engineered to express said Type I and Type II cytokines, or
(c) either the type I cytokine or the type II cytokine is administered as a soluble protein, optionally modified to improve in vivo half-life or delivery to target cells, and the other cytokine is expressed by a recombinant or isolated cell, e.g., a CAR-T cell engineered to express said Type I or Type II cytokine.

In some exemplary embodiments the immunostimulatory combination is used for stimulating an immune response in a subject in need thereof, optionally a synergistic immune response, further optionally a synergistic antitumor immune response, preferably against a solid tumor is stimulated in subject in need thereof which is immunoreplete and is similarly stimulated in a subject in need thereof which lacks sufficient endogenous response from immune cells selected from the group comprising CD8, CD4, NK, and/or ILC2 cells.

In some exemplary embodiments the immunostimulatory combination is used for stimulating an immune response in a subject in need thereof, optionally a synergistic immune response, further optionally a synergistic antitumor immune response, preferably against a solid tumor, optionally wherein the cancer is selected from the group consisting of bladder cancer, bone cancer, brain cancer, breast cancer, cervical cancer, colon cancer, colorectal cancer, desmoid tumor, endometrial cancer, esophageal cancer, fibromatosis, glioblastoma, head and neck cancer, liver cancer, lung cancer, lymphoma, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, testicular cancer, thyroid cancer, urothelial cancer, or a combination of any of the foregoing.

In some exemplary embodiments the immunostimulatory combination is used for the treatment of infectious disease, wherein the infection is selected from the group consisting of viral, bacterial, yeast, fungal, or parasite.

In some exemplary embodiments the invention provides a composition comprising an immunostimulatory combination comprising at least one immune stimulating Type I cytokine, optionally selected from the group consisting of interleukin-21, interleukin-2 (IL-2), and an IL-2 superkine variant (super-2); preferably super-2 and a Type 11 cytokine optionally selected from the group consisting of interleukin-4 (IL-4), interleukin-5 (IL-5), interleukin-25 (IL-25), interleukin-33 (IL-33), and Thymic stromal lymphopoietin (TSLP), or an immunologically active fragment, variant or fusion protein comprising any of the foregoing, preferably IL-33 or an IL-33 fusion protein and/or IL-25 or an IL-25 fusion protein or TSLP or a TSLP fusion protein, which Type I and Type 11 cytokine when administered in combination elicit an additive or synergistic effect on immunity.

In some exemplary embodiments the invention provides a composition comprising an immunostimulatory combination as above described wherein the Type I cytokine comprises or consists of an amino acid sequence at least 80%, at least 85%, at least 90%, at least 92%, at least 94%, at least 95%, at least 96%, at least 97%, at least 98%, at least 99%, or 100% identical to human IL-21, IL-2 or Super-2, optionally modified to enhance (increase) in vivo half-life and/or delivery to target cells.

In some exemplary embodiments the invention provides a composition comprising an immunostimulatory combination as above described wherein the Type II cytokine is selected from the group consisting of interleukin-4 (IL-4), interleukin-5 (IL-5), interleukin-25 (IL-25), interleukin-33 (IL-33), and Thymic stromal lymphopoietin (TSLP), optionally modified to enhance (increase) in vivo half-life and/or delivery to target cells, and immunologically active fragments, variants and fusion proteins comprising any of the foregoing.

In some exemplary embodiments the invention provides a composition comprising an immunostimulatory combination as above described wherein the Type II cytokine has an amino acid sequence at least 80%, at least 85%, at least 90%, at least 92%, at least 94%, at least 95%, at least 96%, at least 97%, at least 98%, at least 99%, or 100% identical to that of IL-4, IL-5, IL-25, IL-33, or TSLP, preferably IL-33, IL-25 or TSLP.

In some exemplary embodiments the invention provides an isolated or recombinant cell which is engineered to express at least one Type I cytokine which comprises IL-21 or an IL-2 like cytokine, optionally selected from the group consisting of interleukin-21 (IL-21) or interleukin-2 (IL-2), further optionally human IL-2; an IL-2 superkine variant (super-2) or other IL-2 variant; and at least one Type II cytokine, optionally selected from the group consisting of interleukin-4 (IL-4), interleukin-5 (IL-5), interleukin-25 (IL-25), interleukin-33 (IL-33), and Thymic stromal lymphopoietin (TSLP), preferably IL-33 or an IL-33 fusion protein, IL-25 or an IL-25 fusion protein or TSLP or a TSLP fusion protein, which cytokines when expressed in combination elicit an additive or synergistic effect on immunity, optionally antitumor immunity, further optionally an immune cell, optionally selected from the group consisting of a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH17 cell, a TH9 cell, a TH22 cell, a follicular helper T cells, an a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, and a dendritic cell.

In some exemplary embodiments the invention provides a recombinant cell as above described, which further expresses a chimeric antigen receptor (CAR) comprising:
(a) an antigen-binding (AB) domain that binds to a target molecule which is expressed on the surface of a tumor, a virus, or virus infected cell or which is overexpressed or aberrantly expressed in cancer or viral infection, or other disease,
(b) a transmembrane (TM) domain,
(c) an intracellular signaling (ICS) domain,
(d) optionally a hinge that joins said AB domain and said TM domain, and
(e) optionally one or more costimulatory (CS) domains.

In some exemplary embodiments the invention provides a recombinant cell expressing a CAR as above described, wherein the CAR binds to a target molecule selected from an antigen, ligand or receptor expressed by tumor or infected cells, optionally a Tyrosinase-related protein 1 (TYRP1), an induced-self antigen from the MIC and/or RAET1/ULBP families which are inducible by stress, malignant transformation, or infection (Rael et al.), or a stress-inducible natural killer (NK) cell ligand (B7H6 et al.).

In some exemplary embodiments the invention provides a recombinant cell expressing a CAR as above described, wherein the AB domain in the CAR comprises an antibody (Ab) or an antigen-binding fragment thereof that binds to said target molecule, wherein said Ab or antigen-binding fragment thereof is optionally selected from a group consisting of a monoclonal Ab, a monospecific Ab, a polyspecific Ab, a humanized Ab, a tetrameric Ab, a tetravalent Ab, a multispecific Ab, a single chain Ab, a domain-specific Ab, a single-domain Ab (dAb), a domain-deleted Ab, an scFc fusion protein, a chimeric Ab, a synthetic Ab, a recombinant Ab, a hybrid Ab, a mutated Ab, CDR-grafted Ab, a fragment antigen-binding (Fab), an F(ab′)2, an Fab′ fragment, a variable fragment (Fv), a single-chain Fv (scFv) fragment, an Fd fragment, a dAb fragment, a diabody, a nanobody, a bivalent nanobody, a shark variable IgNAR domain, a VHH Ab, a camelid Ab, and a minibody.

In some exemplary embodiments the invention provides a recombinant cell expressing a CAR as above described, wherein the TM domain in the CAR is derived from the TM region, or a membrane-spanning portion thereof, of a protein selected from the group consisting of CD28, CD3e, CD4, CD5, CD8, CD9, CD16, CD22, CD33, CD37, CD45, CD64, CD80, CD86, CD134, CD137, CD154, TCRa, TCRb, and CD3z.

In some exemplary embodiments the invention provides a recombinant cell expressing a CAR as above described, wherein the CAR comprises a TM domain is derived from the TM region of CD28, or a membrane-spanning portion thereof.

In some exemplary embodiments the invention provides a recombinant cell expressing a CAR as above described, wherein the CAR comprises an ICS domain derived from a cytoplasmic signaling sequence, or a functional fragment thereof, of a protein selected from the group consisting of CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, and DAP12.

In some exemplary embodiments the invention provides a recombinant cell expressing a CAR as above described, wherein the CAR comprises an ICS domain derived from a cytoplasmic signaling sequence of CD3z, or a functional fragment thereof.

In some exemplary embodiments the invention provides a recombinant cell expressing a CAR as above described, wherein the CAR comprises a hinge derived from CD28 or other costimulatory protein.

In some exemplary embodiments the invention provides a recombinant cell expressing a CAR as above described, wherein the CAR comprises one or more CS domains if present is/are derived from a cytoplasmic signaling sequence, or functional fragment thereof, of a protein selected from the group consisting of CD28, DAP10, 4-1BB (CD137), CD2, CD4, CD5, CD7, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAMI, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and CD83 ligand.

In some exemplary embodiments the invention provides a nucleic acid construct or nucleic acid constructs which separately or in combination comprise a nucleic acid that encodes for a type I cytokine, optionally selected from the group consisting of interleukin-21, interleukin-2 (IL-2), and an IL-2 superkine variant (super-2); preferably super-2, and a nucleic acid that encodes for a type II cytokine, optionally selected from the group consisting of interleukin-4 (IL-4), interleukin-5 (IL-5), interleukin-25 (IL-25), interleukin-33 (IL-33), and Thymic stromal lymphopoietin (TSLP), preferably IL-33 and/or IL-25, which Type I and Type II cytokine when expressed in combination elicit an additive or synergistic effect on immunity.

In some exemplary embodiments the invention provides a nucleic acid construct or nucleic acid constructs as above-described, which further encodes a natural or endogenous receptor or a CAR which binds to target cells, wherein the CAR optionally comprises
(a) an AB domain or receptor that binds to a ligand, antigen or receptor expressed by target cells, e.g., tumor or infected cells,
(b) a transmembrane (TM) domain,
(c) an intracellular signaling (ICS) domain,
(d) optionally a hinge that joins said AB domain and said TM domain, and
(e) optionally one or more costimulatory (CS) domains.

In some exemplary embodiments the invention provides a nucleic acid construct as above-described, which encodes an Ab or Ab fragment, wherein the Ab or Ab fragment binds to an antigen expressed on tumor cells, e.g., bladder cancer, bone cancer, brain cancer, breast cancer, carcinomas, cervical cancer, colon cancer, colorectal cancer, desmoid tumor, endometrial cancer, esophageal cancer, fibromatosis, glioblastoma, head and neck cancer, liver cancer, lung cancer, lymphoma, leukemias, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, testicular cancer, thyroid cancer, urothelial cancer, or a combination of any of the foregoing.

In some exemplary embodiments the invention provides a nucleic acid construct or nucleic acid constructs as above-described, containing a CAR that comprises a TM domain derived from the TM region, or a membrane-spanning portion thereof, of a protein selected from the group consisting of CD28, CD3e, CD4, CD5, CD8, CD9, CD16, CD22, CD33, CD37, CD45, CD64, CD80, CD86, CD134, CD137, CD154, TCRa, TCRb, and CD3z.

In some exemplary embodiments the invention provides a nucleic acid construct or nucleic acid constructs as above-described, containing a CAR that comprises a TM domain derived from the TM region of CD28, or a membrane-spanning portion thereof.

In some exemplary embodiments the invention provides a nucleic acid construct or nucleic acid constructs as above-described, which contain a CAR that comprises a ICS domain derived from a cytoplasmic signaling sequence, or a functional fragment thereof, of a protein selected from the group consisting of CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, and DAP12.

In some exemplary embodiments the invention provides a nucleic acid construct or nucleic acid constructs as above-described, which contain a CAR that comprises at least one or more CS domains is derived from a cytoplasmic signaling sequence, or functional fragment thereof, of a protein selected from the group consisting of CD28, DAP10, 4-1BB (CD137), CD2, CD4, CD5, CD7, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAMI, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and CD83 ligand.

In some exemplary embodiments the invention provides vectors comprising any of the afore-described nucleic acids.

In some exemplary embodiments the vector is selected from a DNA, an RNA, a plasmid, a cosmid, a viral vector, a lentiviral vector, an adenoviral vector, or a retroviral vector.

In some exemplary embodiments the invention provides recombinant or isolated cells comprising at least one nucleic acid or vector as above-described.

In some exemplary embodiments the recombinant or isolated cell as above-described is a non-mammalian cell, optionally selected from the group consisting of a plant cell, a bacterial cell, a fungal cell, a yeast cell, a protozoa cell, and an insect cell.

In some exemplary embodiments the recombinant or isolated cell as above-described is a mammalian cell, optionally selected from the group consisting of a human cell, a monkey cell, a rat cell, and a mouse cell.

In some exemplary embodiments the recombinant or isolated cell as above-described is a stem cell.

In some exemplary embodiments the recombinant or isolated cell as above-described is a primary cell, optionally a human primary cell or derived therefrom.

In some exemplary embodiments the recombinant or isolated cell is a cell line, optionally a hybridoma cell line.

In some exemplary embodiments the recombinant or isolated cell as above-described is an immune cell.

In some exemplary embodiments the recombinant or isolated cell as above-described is MHC+ or MHC−.

In some exemplary embodiments the recombinant or isolated cell as above-described is selected from the group consisting of a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH17 cell, a TH9 cell, a TH22 cell, a follicular helper T cells, an a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, and a dendritic cell.

In some exemplary embodiments the recombinant or isolated cell as above-described is a T cell or T cell progenitor cell.

In some exemplary embodiments the recombinant or isolated cell as above-described is a T cell which has been modified such that its endogenous T cell receptor (TCR) is
(i) not expressed,
(ii) not functionally expressed, or
(iii) expressed at reduced levels compared to a wild-type T cell.

In some exemplary embodiments the recombinant or isolated cell as above-described is activated or stimulated to proliferate and/or to release at least one cytokine, and/or to increase expression of cytokines and/or chemokines and/or to elicit killing of target cells and/or to shift the disease microenvironment from immune suppressive to immune stimulatory, and/or to increase populations of infiltrating immune cells within the disease microenvironment, when it binds to a target molecule.

In some exemplary embodiments the administration of the cell as above-described ameliorates a disease, preferably cancer or infectious disease, optionally a solid tumor.

In some exemplary embodiments the invention provides a population of cells comprising at least one recombinant or isolated cell as above-described.

In some exemplary embodiments the invention provides a pharmaceutical composition comprising (a) a cytokine combination and/or at least one recombinant cell according to any of the foregoing and (b) a pharmaceutically acceptable excipient or carrier.

### BRIEF DESCRIPTION OF THE DRAWINGS

FIG. 1A-F provides exemplary schematics of chimeric antigen receptors (CARs) according to the present disclosure which are described in Example 1. FIG. 1A shows a general schematic of chimeric antigen receptors (CARs) of the present invention. FIGS. 1B-1D shows exemplary schematics of a CAR construct according to the present disclosure, wherein the CAR construct comprises an AB domain, a TM domain, and an ICS domain, and further comprises a hinge that joins the AB and TM domains (FIG. 1B) and one (FIG. 1C) or two (FIG. 1D) costimulatory (CS) domains. FIG. 1E shows exemplary schematics of a CAR-encoding construct that may be included in a vector and comprises a leader sequence (LS) and an exemplary CAR construct as shown in any of FIGS. 1A-1D. FIG. 1F shows exemplary schematics of a vector construct encoding a CAR according to the present disclosure, further comprising an exemplary G3SG3 linker and an exemplary expression/purification marker (Myc-tag). In FIG. 1, different domains (e.g., AB domain, TM domain, etc) are connected via a black line, each representing a potential additional sequence that may or may not be included between domains.

FIG. 2 illustrates a schematic showing various exemplary AB domain constructs of CARs of some embodiments according to Example 1. The first two examples are “TA99scFvHL” (or “TA99 scFv HL”) and “TA99scFvLH” (or “TA99 scFv LH”), which are scFvs derived from murine anti-TYRP1 antibody TA99. The next two examples are “TZ47scFvHL” (or “TZ47 scFv HL”) and “TZ47scFvLH” (or “TZ47 scFv LH”), which are scFvs derived from the humanized version of murine anti-B7H6 antibody TZ47. Optionally, “PB6scFvHL” (or “PB6 scFv HL”) and “PB6scFvLH” (or “PB6 scFv LH”) may be incorporated, which are scFvs derived from the humanized version of murine anti-B7H6 antibody PB6. The last two examples are “NKG2DscFvHL” (or “NKG2D scFv HL”) and “NKG2DscFvLH” (or “NKG2D scFv LH”), which are scFvs that target Rael, a stress ligand that is expressed naturally on MC38 colon tumors.

FIG. 3A-C contain schematics of various exemplary CAR constructs of some embodiments according to Example 1. In the exemplary constructs in FIG. 3A, one of the AB domains shown in FIG. 2 is used as the AB domain, CD8αH is used as the hinge, CD8αTM is used as the TM domain, CD28CS is used as the CS domain, and CD3zICS is used as the ICS domain. In FIG. 3B, one of the AB domains shown in FIG. 2 is used as the AB domain, CD8αH is used as the hinge, CD8αTM is used as the TM domain, 41BBCS is used as the CS domain, and CD3zICS is used as the ICS domain. In FIG. 3C, one of the AB domains shown in FIG. 2 is used as the AB domain, CD8αH is used as the hinge, CD8αTM is used as the TM domain, DAP10CS is used as the CS domain, and CD3zICS is used as the ICS domain. CD8αH is the hinge derived from mouse CD8α. CD8αTM is the TM domain derived from mouse CD8α. CD28CS is the CS region derived from a cytoplasmic signaling sequence of murine or human CD28. CD3zICS is the ICS domain derived from a murine or human CD3 zeta. Any of the CAR constructs described in this figure or in this application may be used with LS, G3SG3 linker, and/or Myc-tag, as shown in FIGS. 1E and 1F. In one embodiment, the LS is derived from a mouse CD8 signal peptide. In FIG. 3, although no connecting black lines are shown between different domains, additional sequence(s) may or may not be included between domains, e.g., other CS domains.

FIG. 4A-B provide exemplary schematics of immunostimulatory cytokine combinations according to the present disclosure which are described in Example 1. FIG. 4A shows a general schematic of unlinked Type I and Type II cytokines of the present invention.

FIG. 4B shows an exemplary schematic of a cytokine construct according to the present disclosure, wherein the cytokine construct comprises a Type I cytokine domain and a Type II cytokine domain linked by a self-cleaving peptide sequence domain.

FIG. 5 contains schematics of various exemplary cytokine constructs of some embodiments of the invention according to Example 1. In the first exemplary construct, the Type I cytokine is the interleukin 2 variant, super-2, the Type II cytokine is interleukin 33 (IL-33) and the linker is the self-cleaving peptide sequence T2A, which is derived from Thosea asigna virus 2A. In the second exemplary construct, the Type I cytokine is IL-2 fused to mouse serum albumin (MSA/IL-2), the Type II cytokine is IL-33, and the linker is absent. In the third exemplary construct, the Type I cytokine is MSA/IL-2, the Type II cytokine is IL-25, and the linker is absent.

FIG. 6 shows a flow chart illustrating one of many possible methods for manufacturing isolated recombinant CAR-expressing cells, according to Example 1, that may be used for in vitro or in vivo assays.

FIG. 7A-B contains the results from an in vivo efficacy test described in Example 2. C57BL/6J mice harboring intradermal B16F10 tumors were treated with T cells expressing (i) Super-2-T2A-IL-33 cytokine construct and TA99 CAR, (ii) TA99 CAR only, (iii) Super-2 and TA99 CAR, (iv) IL-33 and TA99 CAR, (v) TA99 CAR only, or (vi) no treatment. FIG. 7A shows a graph of average tumor volume over 17 days post-treatment. FIG. 7B shows a graph of percent survival post injection.

FIG. 8 contains the results from an in vivo efficacy test described in Example 3. C57BL/6J mice harboring metastatic lung B16F10 tumors were treated with T cells expressing (i) Super-2-T2A-IL-33 cytokine construct and TA99 CAR, (ii) TA99 CAR only, or (iii) no treatment. FIG. 8 shows mouse lungs harvested 15 days post-treatment and a graph of average tumor foci count. P value: ****p<0.0001, ***p<0.001, **p<0.01, *p<0.05.

FIG. 9 contain results from an in vivo efficacy test described in Example 4. C57BL/6J mice harboring subcutaneous B16F10 tumors were treated with (i) a combination of recombinant cytokines IL-33 and MSA/IL-2, (ii) IL-33, (iii) MSA/IL-2, or (iv) PBS control. FIG. 9 shows graphs of average tumor weight and area over 20 days post-treatment.

FIG. 10A-B contains results from an in vivo efficacy test described in Example 5. Rag2-deficient (Rag2−/−) mice harboring subcutaneous B16F10 tumors were treated with (i) a combination of B16F10-expressing cytokines IL-33 and IL-2, (ii) IL-33, (iii) IL-2, or (iv) PBS control. Additional cohorts were treated with (i) IL-2+anti-NK1.1 and (ii) combination of B16F10-expressing cytokines IL-33 and IL-2+anti-NK1.1. Additional cohorts were treated with (i) a combination of B16F10-expressing cytokines IL-2 and IL-25, (ii) IL-2, (iii) IL-25, (iv) IL-15, or (v) PBS control. FIG. 10A shows graphs of average tumor weight and tumor area 25 days after treatment are shown. FIG. 10B shows graphs of average tumor weight at day 18 and area over 40 days post-treatment.

FIG. 11 contains results from an in vitro efficacy test described in Example 6. In vitro expression of Type II cytokine, IL-33, by TZ47 CAR T cells was assayed by ELISA. The graph shows pg/mL of IL-33 produced when TZ47 CAR T cells specific for B7H6 were incubated with various amounts of plate-bound recombinant hB7H6.

FIG. 12A-B contains an exemplary schematic of one of many possible CAR constructs comprising an antibody domain and optionally additional domains as shown, which may be co-expressed in cells along with immunostimulatory cytokine constructs of the present invention according to Example 1. FIG. 12A contains an exemplary amino acid sequence of an anti-TYRP1 CAR construct of the present invention, wherein the CAR construct comprises a signal peptide LS, a TA99 scFV AB domain, a G3SG3 linker, an exemplary expression/purification marker (Myc-tag), CD8α hinge and TM domain, a CD28 CS domain, and a CD3ζ ICS domain. FIG. 12B shows an exemplary schematic of a CAR-encoding nucleic acid construct, encoding for the CAR of FIG. 12A, that may be included in a vector.

FIG. 13A-B contains an exemplary schematic of one of many possible immunostimulatory cytokine constructs comprising a Type 1 cytokine domain and a Type 2 cytokine domain optionally linked by a self-cleaving peptide sequence domain and optionally containing additional domains and motifs as shown prepared according to Example 1. FIG. 13A contains an exemplary amino acid sequence of a cytokine construct of the present invention, wherein the construct comprises a signal sequence (SS1), a Type 1 cytokine (Super-2), a self-cleaving peptide sequence (T2A), a second signal sequence (SS2), a Type 2 cytokine (IL-33), and a GGSGGS motif linking V5 and His6 tags for detection and purification. FIG. 13B shows an exemplary schematic of a cytokine encoding nucleic acid construct, encoding for the immunostimulatory cytokine construct shown in FIG. 13A, that may be included in a vector.

FIG. 14 displays a graph of human B7H6 expression in various tumors created with data from Human Protein Atlas according to Example 10.

FIG. 15 contains results from an in vitro cytotoxicity assay described in Example 7, wherein Luciferase expressing B16F10 mouse melanoma (target) cells were treated with CAR T (effector) cells expressing (i) a Super-2-T2A-IL-33 cytokine construct and TA99 CAR, (ii) Super-2 and TA99 CAR, (iii) IL-33 and TA99 CAR, and (iv) TA99 CAR only. Untreated luciferase expressing B16F10 cells were used as a negative control. A graph of luminescence, which correlates to target cell numbers, over a range of ratios of effector cells to target cells is shown.

FIG. 16A-B contains results from an in vitro assay described in Example 8, wherein natural killer (IL2Ra−/− NK1.1) cells were cultured with IL-2 and type 2 innate lymphoid cells (wt ILC2) or IL2Ra−/− ILC2 cells and stained with antibodies to detect CD25, NK1.1 or cytoplasmic dye Cell Trace Violet. FIG. 16A shows representative images of sorted cells with staining for the innate NK cell marker, (NK1.1, green), IL-2 receptor alpha chain (CD25, red), cell trace violet (violet), and image overlays. FIG. 16B shows flow results of cytometry analysis of cultured cells based on Granzyme C (GzmC) and cell trace violet (CTV) staining.

FIG. 17 contains results from an in vivo experiment described in Example 9 wherein C57BL/6 mice were subjected to subcutaneous inoculation with 1×10\hat{ }6 B16F10 tumor cells. Tumors of B16F10 expressing IL-2 were resected 7 days after inoculation. Tumor sections were stained with antibodies against Thy1 (red), CD3 (cyan), and NK1.1 (green). FIG. 17 shows representative images of immunohistochemical staining of an ILC2 cell (NK1.1−Thy1+CD3−) interacting with a T cell (NK1.1−Thy1+CD3+) and an NK or ILC1 cell (NK1.1+ Thy1+CD3−). Scale bar: 5 μm.

FIG. 18 displays graphs of data described in Example 10, showing percent survival over time for patients with endometrial cancers (created with data from Human Protein Atlas). FIG. 18 (top) shows survival differences in endometrial cancer patients expressing higher IL-33 transcripts and those with low IL-33 expression. FIG. 18 (bottom) shows survival differences in endometrial cancer patients expressing a combination of IL-2 and CD8 T cells and ILC2-associated transcripts.

FIG. 19 displays graphs of tumor volume (top) and percent survival (bottom) over time of B16F10 tumor-bearing mice treated with CAR T cells engineered to express Super2 in combination with either IL4, IL5, IL25, TSLP, or IL33 according to in vivo experiments described in Example 11.

FIG. 20 displays graphs of tumor volume over time (top) and final tumor mass 19 days post inoculation (bottom) of B16F10 tumor-bearing mice treated with wild type (WT), IFNγ deficient (IFNγ KO), or perforin deficient (Prf KO) CAR T cells engineered to express Super2 in combination with IL33 according to in vivo experiments described in Example 12.

FIG. 21 displays graphs of tumor volume over time (top) and final tumor mass (bottom) of B16F10 tumor-bearing mice treated with (i) CAR T cells engineered to express Super2 and IL33 or (ii) T cells expressing Super2 and IL33 without CAR expression according to in vivo experiments described in Example 13.

FIG. 22A-B contains graphs of tumor infiltrating leukocytes in B16F10 tumor-bearing mice treated with CAR T cells engineered to express Super2 and IL33 according to Example 14. FIG. 22A displays graphs of flow cytometry counts of tumor infiltrating leukocytes (CD8, CD4, NK, and ILC2 cells) per milligram of tumor. FIG. 22B displays a graph of flow cytometry counts of tumor infiltrating CAR T cells per milligram of tumor.

FIG. 23A-B contains graphs of tumor volume over time in immune replete (WT) and immune depleted recipient mice bearing B16F10 tumors and treated with CAR T cells expressing Super 2 and IL33 according to Example 15. FIG. 23A displays graphs of tumor volume in CD8 deficient (left) and NK depleted mice (right). FIG. 23B displays graphs of tumor volume in CD4 depleted (top) and RORα deficient mice (bottom).

FIG. 24A-D contains results from single cell RNA sequencing analyses of tumor infiltrating leukocytes (TIL) from B16F10 tumor-bearing mice treated with CAR T cells engineered to express Super2 and IL33 according to Example 16. FIG. 24A displays 2-D UMAP clustering of overall TIL populations (top) and cell clustering differentiated by CAR treated tumors vs untreated tumors (bottom). FIG. 24B displays a heatmap of gene expression in each TIL cell type cluster. FIG. 24C displays graphs of frequency for each TIL cell type (left) and frequency of monocytes, dendritic cells, and B cells (right) for each treatment condition.

FIG. 24D displays UMAP clustering (top) and gene expression values for monocytes, dendritic cells, and B cells within the tumor.

FIG. 25A-B contains graphs of tumor volume over time and final mass of tumors extracted from mice bearing (i) MC38 colon tumors or (ii) B16F10 tumors expressing the NK cell ligand B7H6 following treatment according to Example 17. FIG. 25A displays MC38 colon tumor volume (top) and final mass (bottom) following treatment with NKG2D CAR T cells engineered to express Super2 and IL33. FIG. 25B displays B7H6 expressing tumor volume following treatment with TZ47 CAR T cells engineered to express Super2 and IL33.

FIG. 26A-B contains an exemplary schematic of one of many possible immunostimulatory cytokine constructs comprising a Type 1 cytokine domain and a Type 2 cytokine domain optionally linked by a self-cleaving peptide sequence domain and optionally containing additional domains and motifs as shown, prepared according to Example 1. FIG. 26A contains an exemplary amino acid sequence of said immunostimulatory cytokine construct comprising Super2 and IL25. FIG. 26B contains an exemplary nucleic acid construct which encodes for said immunostimulatory cytokine construct and may be included in a vector.

FIG. 27A-B contains an exemplary schematic of one of many possible CAR constructs comprising an antibody domain and optionally additional domains as shown, which may be co-expressed in cells along with immunostimulatory cytokine constructs of the present invention according to Example 1. FIG. 27A contains an amino acid sequence of an exemplary anti-B7H6 (TZ47) CAR construct. FIG. 27B contains a nucleic acid sequence which encodes for said exemplary anti-B7H6 (TZ47) CAR construct and may be included in a vector.

FIG. 28A-B contains an exemplary schematic of one of many possible CAR constructs comprising an antibody domain and optionally additional domains as shown, which may be co-expressed in cells along with immunostimulatory cytokine constructs of the present invention according to Example 1. FIG. 28A contains an amino acid sequence of an exemplary anti-Rael CAR construct comprising mouse NKG2D full length type 2 membrane protein. FIG. 28B contains a nucleic acid sequence which encodes for said exemplary anti-Rael CAR construct and may be included in a vector.

FIG. 29A-C contains the results of in vivo experiments in a rodent xenograft model showing the efficacy of Super2+IL-33 CAR T cells against medium and large tumors. The mice were administered a single dose of Super2+IL33 CAR T cells on day 6-, 11- or 14-days post intradermal B16F10 tumor inoculation results in reduced tumor growth. FIG. 29A contains the experimental plan. FIG. 29B shows B16F10 tumor volume over time. FIG. 29C shows individual tumor volumes in mice with no treatment or treated on day 6, 11 or 14 with 7×10⁶ Super2+IL33 CAR T cells.

FIG. 30A-C contains the results of in vivo experiments demonstrating that CAR T cells expressing Super2+IL-33 have improved expansion and remain in B16F10 tumors longer. In the experiments CAR T cells expressing TA99 CAR alone or with Super2+IL-33 were tracked in vivo to determine their abundance and localization. FIG. 30A contains the experimental plan showing that B6 albino mice were inoculated i.d. with B16F10 and treated with TA99 CAR T cells with or without Super2+IL-33 and that also expressed luciferase to visualize their in vivo abundance and localization by IVIS imaging on indicated days. FIG. 30B shows the Quantification of TA99 CAR T qcell alone or with Super2+IL33 in whole mice. FIG. 30C shows representative IVIS images at indicated times.

FIG. 31 further contains enlarged images of representative IVIS images at indicated times.

FIG. 32A-C contains experimental results showing that TA99 CAR constructs lacking function CD28 and CD3 zeta signaling motifs retain considerable efficacy. The experimental data indicate that truncating the TA99 CAR signaling domains (CD28 and CD3zeta) has minimal effect on Super2+IL-33 CAR T cell efficacy against B16F10 tumor. FIG. 32A contains a schematic of the CAR constructs. FIG. 32B shows B16F10 tumor volume over time in non-treated mice or mice treated with Super2+IL33 full length TA99 CAR or Tailless TA99 CAR. FIG. 32C contains the amino acid sequence of TA99 CAR lacking a tail. The yellow highlighting is the anti-GP75 scFv sequence, the Aqua is the CD8 transmembrane sequence and the red comprises the truncated CD28 cytoplasmic domain.

FIG. 33 contains experimental results showing that Super2+IL-33 TA99 CAR T cell treatment induces changes in regulatory T cells gene expression. In these experiments regulatory T cells were isolated from tumors from non-treated mice or mice treated with Super2+IL-33 TA99 CAR T cells. As shown single cell RNA sequencing was conducted on tumor infiltrating cells from non-treated or Super2+IL-33 TA99 CAR T cell treated mice. Gene expression in regulatory T cells were compared and found to be highly different. Satb1, Il2ra, Hif1a, Dgat1 and Il1rl1 were found to be highly enriched in Tregs isolated from Super2+IL-33 TA99 CAR T cell treated mice.

FIG. 34A-B contains experimental results showing that human T cells expressing TZ47 (anti-B7H6) CAR and are equally effective at killing K562 tumors expressing B7H6 tumor antigen with or without Super2+IL-33 expression. In the experiments human T cells expressing TZ47 (anti-B7H6) CAR and Super2+IL-33 are shown to be equally effectively at killing K562 tumors expressing B7H6 tumor antigen. FIG. 34A shows that human PBMCs were activated and transduced with TZ47 anti-B7H6 CAR co-expressing mouse CD19 (mCD19) and Super2+IL-33 co-expressing GFP) and wherein the expression of mCD19 and GFP were detected by flow cytometry. FIG. 34B shows that human T cells transduced with mCD19 virus with no CAR, TZ47 CAR only or TZ47 CAR and Super2+IL-33 were co-cultured at indicated ratios with K562 tumor cells expressing luciferase for 24 hours before K562 viability was determined. Luminescence signal correlates with tumor cell viability.

FIG. 35A-C contains experimental results showing that the expression of Super2+IL-33 does not alter the transcriptional profile or subset distribution of human TZ47 (anti-B7H6) CAR T cells. The experiments comprised single cell analysis of human TZ47 CAR T cells with or without Super2+IL-33 expression. FIG. 35A shows UMAP projection of 6 T cell clusters from human CAR T cells. FIG. 35B shows an overlay of cells from TZ47 CAR only versus Super2+IL-33 TZ47 CAR shows similar representation in T cell clusters. FIG. 35C shows that T cell cluster identity was determined using SingleR with clusters 0, 1, 2 and 4 being representative of effector memory CD8 T cells and cluster 2 and 5 representative of CD4 Th1 cells.

### DETAILED DESCRIPTION OF THE INVENTION

### Definitions

Prior to discussing the invention in detail, the following definitions are provided.

In the specification above and in the appended claims, all transitional phrases such as “comprising,” “including,” “having,” “containing,” “involving,” “composed of,” and the like are to be understood to be open-ended, namely, to mean including but not limited to. Only the transitional phrases “consisting of” and “consisting essentially of” shall be closed or semi-closed transitional phrases, respectively.

It must also be noted that, unless the context clearly dictates otherwise, the singular forms “a,” “an,” and “the” as used herein and in the appended claims include plural reference. Thus, the reference to “a cell” refers to one or more cells and equivalents thereof known to those skilled in the art, and so forth. Unless defined otherwise, all technical and scientific terms used herein have the same meanings as commonly understood by a person of skilled in the art.

It should be understood that, unless clearly indicated otherwise, in any methods disclosed or claimed herein that comprise more than one step, the order of the steps to be performed is not restricted by the order of the steps cited.

The term “about” or “approximately” as used herein when referring to a numerical value, such as of weight, mass, volume, concentration, or time, should not be limited to the recited numerical value but rather encompasses variations of +/−10% of a given value.

The term “cytokines” as used herein refers to a broad category of small proteins that are involved in cell signaling. Generally, their presence has some effect on the behavior of cells around them. Cytokines may be involved in autocrine signaling, paracrine signaling and/or endocrine signaling as immunomodulating agents. Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, epithelial cells, and various stromal cells. “Chemokines” are a family of cytokines generally involved in mediating chemotaxis.

A “Type-1 cytokine” herein refers to a cytokine produced by CD8 T cell or Th1 T-helper cells. Type-1 cytokines include IL-2 (IL2) and modified forms thereof such as Super-2, IFN-gamma (IFN-G), IL-12 (IL12), IL-15 (IL15), IL-21 (IL21) & TNF-beta (TNF-b). In general, type 1 cytokines favor the development of a strong cellular immune responses.

A “Type-2 cytokine” herein refers to a cytokine produced by Th2 T-helper cells. Type 2 cytokines include TSLP, IL-25, IL-33, IL-1(IL1), IL-4 (IL4), IL-5 (IL5), IL-6 (IL6), IL-10 (IL10), et al. In general, type 2 cytokines favor a strong humoral immune response.

“Interleukin-2”, or “IL-2” or “IL2” is an interleukin, which comprises a type of cytokine signaling molecule which is immunostimulatory and which acts as a growth factor for a wide range of leukocytes, including T cells and natural killer (NK) cells and which regulates the activities of white blood cells (leukocytes, often lymphocytes) that are responsible for immunity. IL-2 is a 15.5-16 kDa protein and is part of the body's natural response to microbial infection, and in discriminating between foreign (“non-self”) and “self”. IL-2 mediates its effects by binding to IL-2 receptors, which are expressed by lymphocytes. The major sources of IL-2 are activated CD4+ T cells and activated CD8+ T cells. On activated T cells, IL-2 signals through a quaternary ‘high affinity’ receptor complex consisting of IL-2, IL-2Rα (termed CD25), IL-2Rβ and IL-2Rγ. Naive T cells express only a low density of IL-2Rβ and IL-2Rγ, and are therefore relatively insensitive to IL-2, but acquire sensitivity after CD25 expression, which captures the cytokine and presents it to IL-2Rβ and IL-2Rγ. IL-2 finds known use as a therapeutic agent for a variety of immune disorders ranging from AIDS to cancer.

An “Interleukin-2-like cytokine” or “IL-2-like cytokine” or “IL2-like cytokine” herein includes all species forms and variants of IL-2, e.g., rodent, human, non-human primate forms of IL-2 and immunologically active fragments and variants thereof. Specifically an “IL-2-like cytokine” herein includes all naturally occurring and engineered variants of IL-2, e.g., human IL-2 variants such as Super-2, and other modified forms of Il-2 such as desleukin (branded as Proleukin), Interking which comprises a recombinant IL-2 with a serine at residue 125, sold by Shenzhen Neptunus, and Neoleukin 2/15, which is a computationally designed mimic of IL-2 that was designed to avoid common side effects.

“Super-2” or “SUPER2” or “Super2” or “superkine” herein specifically refers to a modified form of IL-2, generally human IL-2, engineered to eliminate or reduce the functional requirement of IL-2 for CD25 expression. Super-2 possesses increased binding affinity for IL-2Rβ as compared to wild-type human IL-2 (hIL-2). Super-2 compared to IL-2 more potently induces expansion of cytotoxic T cells, leading to improved antitumor responses in vivo, and less expansion of T regulatory cells. (See Levin et al., “Exploiting a natural conformational switch to engineer an interleukin-2 ‘superkine’”, Nature, 2012, Mar. 25; 484(7395):529-33; Carmenate et al., “Human IL-2 Mutein with Higher Antitumor Efficacy Than Wild Type IL-2”, J Immunol Jun. 15, 2013, 190 (12) 6230-6238; Mitra e al., “Interleukin-2 Activity Can Be Fine Tuned with Engineered Receptor Signaling Clamps”, Immunity 42(5):826-838 March 2015, et al.) Different super2 cytokines have been reported including those comprising one or more amino acid substitutions relative to the endogenous or wild-type human IL-2 sequence that increase IL-2Rβ binding affinity, wherein the one or more amino acid substitutions in wild-type or endogenous human IL-2 that increase IL-2Rβ binding affinity optionally are selected from the group consisting of: Q74N, Q74H, Q74S, L80F, L8V, R81D, R81T, L85V, I86V, I89V, and I93V; L80F, R81D, L85V, I86V, and I92F; Q74N, L80F, R81D, L85V, I86V, I89V, and I92F; Q74N, L80V, R81T, L85V, I86V, and I92F; Q74H, L80F, R81D, L85V, I86V, and I92F; Q74S, L80F, R81D, L85V, I86V, and I92F; Q74N, L80F, R81D, L85V, I86V, and I92F; and Q74S, R81T, L85V, and I92F (all of the foregoing mutations numbered in accordance with wild-type hIL-2) (See U.S. Pat. No. 10,150,802). The sequence of wild-type mature human IL-2 from U.S. Pat. No. 10,150,802 is shown below:

Ala Pro Thr Ser Ser Ser Thr Lys Lys Thr Gln Leu
Gln Leu Glu His Leu Leu Leu Asp Leu Gln Met Ile
Leu Asn Gly Ile Asn Asn Tyr Lys Asn Pro Lys Leu
Thr Arg Met Leu Thr Phe Lys Phe Tyr Met Pro Lys
Lys Ala Thr Glu Leu Lys His Leu Gln Cys Leu Glu
Glu Glu Leu Lys Pro Leu Glu Glu Val Leu Asn Leu
Ala Gln Ser Lys Asn Phe His Leu Arg Pro Arg Asp
Leu Ile Ser Asn Ile Asn Val Ile Val Leu Glu Leu
Lys Gly Ser Glu Thr Thr Phe Met Cys Glu Tyr Ala
Asp Glu Thr Ala Thr Ile Val Glu Phe Leu Asn Arg
Trp Ile Thr Phe Cys Gln Ser Ile Ile Ser Thr Leu
Thr

“Interleukin 33” or “IL-33” or “IL33” herein refers to a member of the IL-1 family that potently drives production of T helper-2 (Th2)-associated cytokines (e.g., IL-4). IL33 is a ligand for ST2 (IL1RL1), an IL-1 family receptor that is highly expressed on Th2 cells, mast cells and group 2 innate lymphocytes.

“MSA/IL-2” as used herein, refers to the Type 1 cytokine, IL-2 fused to mouse serum albumin, which has an extended half-life in serum (Cancer Cell. 2015 Apr. 13; 27(4): 489-501. doi: 10.1016/j.ccell.2015.03.004).

“Interleukin 25” or “IL-25” or “IL25”, also known as IL-17E, herein, refers to a member of the IL-17 cytokine family and is a protein encoded in humans by the IL25 gene on chromosome 14. IL-25 is a pro-inflammatory cytokine, which is expressed by multiple immune cells including T cells, dendritic cells, macrophages, and stimulates Th2-type immune response.

“TYRP1” as used herein, also known as Tyrosinase related protein 1, TRP, TRP1, CAS2, CATB, GP75, OCA3, TYRP, or b-PROTEIN refers to the gp75 glycoprotein (Tyrp1/gp75), a melanoma associated antigen protein involved in the pigmentary machinery of the melanocyte and often used as differentiation marker, with roles in malignant melanocyte and melanoma progression (Ghanem G. et al., Mol Oncol. 2011 April, 5(2): 150-155. doi: 10.1016/j.molonc.2011.01.006). Human Tyrp1 is encoded by the TYRP1 gene. The gene is located on chromosome 9 (9p23). Human Tyrp1 has an amino acid sequence provided as UniProtKB/Swiss-Prot Accession: P17643.2, or the equivalent residues from a non-human species, e.g., mouse, rodent, monkey, ape and the like.

“B7H6” as used herein, also known as NCR3LG1, B7-H6; or DKFZp686024166 is natural killer cell cytotoxicity receptor 3 ligand 1. B7H6 belongs to the B7 family and is selectively expressed on tumor cells. Interaction of B7H6 with the natural killer (NK) cell receptor, NKp30, results in NK cell activation and cytotoxicity. B7H6 is not typically expressed in normal human tissues but is expressed on a subset of human tumor cells, emphasizing that the expression of stress-induced self-molecules associated with cell transformation serves as a mode of cell recognition in innate immunity (Brandt et al., J Exp Med. 2009 Jul. 6; 206(7):1495-503. doi: 10.1084/jem.20090681). Human B7H6 is encoded by the NCR3LG1 gene and is located on chromosome 11p15.1. Human B7H6 has an amino acid sequence provided as UniProtKB/Swiss-Prot Accession: Q68D85.1, or the equivalent residues from a non-human species, e.g., rodent, monkey, ape and the like.

The term “antibody” or “Ab,” or “immunoglobulin” as used herein, refers to an immunoglobulin molecule which specifically binds with an antigen. Typically, a full-size Ab comprises two pairs of chains, each pair comprising a heavy chain (HC) and a light chain (LC). A HC typically comprises a variable region and a constant region. A LC also typically comprises a variable region and constant region. The variable region of a heavy chain (VH) typically comprises three complementarity-determining regions (CDRs), which are referred to herein as CDR 1, CDR 2, and CDR 3 (or referred to as CDR-H1, CDR-H2, CDR-H3, respectively). The constant region of a HC typically comprises a fragment crystallizable region (Fc region), which dictates the isotype of the Ab, the type of Fc receptor the Ab binds to, and therefore the effector function of the Ab. Fc receptor types include, but are not limited to, FcaR (such as FcaRI), Fca/mR, FceR (such as FceRI, FceRII), and FcgR (such as FcgRI, FcgRIIA, FcgRIIB1, FcgRIIB2, FcgRIIIA, FcgRIIIB) and their associated downstream effects are well known in the art. The variable region of a light chain (VL) also typically comprises CDRs, which are CDR 1, CDR 2, and CDR 3 (or referred to as CDR-L1, CDR-L2, CDR-L3, respectively). In some embodiments, the antigen is TYRP1 or B7H6. Antibodies can be intact immunoglobulins derived from natural sources or from recombinant sources. A portion of an antibody that comprises a structure that enables specific binding to an antigen is referred to “antigen-binding fragment,” “AB domain,” “antigen-binding region,” or “AB region” of the Ab.

The term “antibody fragment” or “Ab fragment” as used herein refers to any portion or fragment of an Ab, including intact or full-length Abs that may be of any class or sub-class, including IgG and sub-classes thereof, IgM, IgE, IgA, and IgD. The term encompasses molecules constructed using one or more portions or fragments of one or more Abs. An Ab fragment can be immunoreactive portions of intact immunoglobulins. The term is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), diabodies, and single domain antibodies (e.g., sdAb, sdFv, nanobody) fragments. The term also encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In a specific embodiment, the antibody fragment is a scFv.

A “heavy chain” or “HC” of an Ab, as used herein, refers to the larger of the two types of polypeptide chains present in all Ab molecules in their naturally occurring conformations.

A “light chain” or “LC” of an Ab, as used herein, refers to the smaller of the two types of polypeptide chains present in all Ab molecules in their naturally occurring conformations. Kappa and lambda light chains refer to the two major antibody light chains.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one genes and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated, synthesized, or can be derived from a biological sample, or might be macromolecule besides a polypeptide. Such a biological sample can include, but is not limited to a tissue sample, a cancer tissue sample, a tumor tissue sample, a leukemic cell sample, an inflamed tissue sample, and a cell or a fluid with other biological components. In some embodiments, the antigen is TYRP1 or B7H6.

The term “antigen-binding domain” or “AB domain” refers to a portion of the CAR constructs of the present invention, that portion comprises a structure that enables specific binding of the anti-TYRP1 agents to TYRP1, or the specific binding of the anti-B7H6 agents to B7H6. When the anti-TYRP1 or anti-B7H6 agent is an Ab, the AB domain may comprise the variable region of the Ab or a portion of the variable region, such as the CDRs. When the anti-TYRP1 or anti-B7H6 agent is an antigen-binding Ab fragment or an antibody-drug conjugate, the AB domain may comprise the variable region or a portion of the variable region, such as the CDRs, of the Ab that the anti-TYRP1 or anti-B7H6 agent is derived from. When the anti-TYRP1 or anti-B7H6 agent is a chimeric antigen receptor (CAR), the AB domain may be one or more extracellular domains of the CAR which have specificity for TYRP1 or B7H6. When the AB domain is derived from an Ab or antigen-binding Ab fragment, the AB domain may comprise the AB domain, such as the variable region or a portion of the variable region, such as the CDRs, of the Ab or antigen-binding Ab fragment that it is derived from. In some embodiments, the AB domain of an anti-TYRP1 or anti-B7H6 agent of the present invention is scFv. In some embodiments, the AB domain may comprise or be derived from a naturally existing molecule that binds to TYRP1 or B7H6 or binding portion of said molecules. Examples of such a molecule include, but are not limited to, NKp30, which binds B7H6, and GIPC PDZ domain containing family, member 1 (GIPC1), which interacts with TYRP1.

The term “bind” refers to an attractive interaction between two molecules that results in a stable association in which the molecules are in close proximity to each other. The result of molecular binding is sometimes the formation of a molecular complex in which the attractive forces holding the components together are generally non-covalent, and thus are normally energetically weaker than covalent bonds.

The term “cancer” refers to a disease characterized by the uncontrolled growth of aberrant cells. Cancer cells can spread locally or through the bloodstream and lymphatic system to other parts of the body. Examples of various cancers relevant to the present invention include, but are not limited to bladder cancer, bone cancer, brain cancer, breast cancer, carcinomas, cervical cancer, colon cancer, colorectal cancer, desmoid tumor, endometrial cancer, esophageal cancer, fibromatosis, glioblastoma, head and neck cancer, liver cancer, lung cancer, lymphoma, leukemias, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, testicular cancer, thyroid cancer, urothelial cancer, or a combination of any of the foregoing.

The term “infectious disease” refers to disorders or illnesses caused by infection wherein pathogenic organisms (e.g., virus, bacteria, yeast, fungus, or parasite) invade the body's tissues, multiply, release toxins, and cause reaction within the host tissues. Infectious diseases may be transmissible or communicable.

The term “CD8α” refers to the T-cell surface glycoprotein CD8 alpha chain on the Cluster of Differentiation 8. CD8α is one of the proteins expressed on cytotoxic T cells that functions as a coreceptor for the T cell receptor (TCR) and plays a role in T-cell signaling and facilitating cytotoxic T cell antigen interactions. Human CD8α is encoded by the CD8A gene. The gene is located on chromosome 2 (2p12). Human CD8α protein may have at least 85, 90, 95, 96, 97, 98, 99 or 100% identity to the amino acid sequence provided by UniProtKB/Swiss-Prot Accession: P01732, or a fragment thereof that has stimulatory activity. The term “CD8α transmembrane domain,” also referred to as “CD8α TM domain” or “CD8αTM” refers to the amino acid residues derived from the transmembrane domain of CD8α. In some embodiments, “CD8αTM” may be derived from a human or non-human species, e.g., mouse, rodent, monkey, ape and the like. In some embodiments, “CD8α TM domain” may be encoded by the nucleic acid sequence provided in the figures. The term “CD28 hinge” as used herein refers to amino acid residues that may be used to join two domains or two portions within a domain in CARs of some of the embodiments. In some embodiments, “CD8α hinge” comprises the sequence in the figures or the equivalent residues from a non-human species, e.g., mouse, rodent, monkey, ape and the like.

The term “CD28” refers to the protein Cluster of Differentiation 28, one of the proteins expressed on T cells that provide co-stimulatory signals required for T cell activation and survival. Human CD28 protein may have at least 85, 90, 95, 96, 97, 98, 99 or 100% identity to NCBI Reference No: NP_006130 or a fragment thereof that has stimulatory activity. The term “CD28 transmembrane domain,” also referred to as “CD28 TM domain” or “CD28TM” refers to the amino acid residues derived from the transmembrane domain of CD28. In some embodiments, “CD28TM” may be derived from a human or non-human species, e.g., mouse, rodent, monkey, ape and the like. The term “CD28 hinge” as used herein refers to amino acid residues that may be used to join two domains or two portions within a domain in CARs of some of the embodiments.

The term “CD28 costimulatory domain,” also referred to as “CD28 CS domain” or “CD28CS,” refers to the amino acid residues derived from the cytoplasmic domain of CD28. In some embodiments, “CD28CS” comprises the sequence provided in the figures or the equivalent residues from a non-human species, e.g., mouse, rodent, monkey, ape and the like. In some embodiments, “CD28 CS domain” may be encoded by the nucleic acid sequence provided in the figures.

The term “CD3 zeta,” or alternatively, “zeta,” “zeta chain,” “CD3-zeta,” “CD3z,” “TCR-zeta,” or “CD247,” is a protein encoded by the CD247 gene on chromosome 1, with gene location 1q24.2, in humans. CD3 zeta, together with T cell receptor (TCR) and CD3 (a protein complex composed of a CD3 gamma, a CD3 delta, and two CD3 epsilon), forms the TCR complex. Human CD3 zeta may have an amino acid sequence provided as NP_000725 or NP_932170, or the equivalent residues from a non-human species, e.g., mouse, rodent, monkey, ape and the like. The term “CD3 zeta intracellular signaling domain,” or alternatively “CD3 zeta ICS domain” or a “CD3zICS,” is defined as the amino acid residues from the cytoplasmic domain of the CD3 zeta chain, or functional derivatives thereof, that are sufficient to functionally transmit an initial signal necessary for T cell activation.

The term “Chimeric Antigen Receptor” or alternatively a “CAR” refers to a set of polypeptides, typically two in the simplest embodiments, which when in an immune effector cell, provides the cell with specificity for a target cell, and with intracellular signal generation. In some embodiments, a CAR comprises at least an extracellular antigen binding domain (AB domain), a transmembrane domain (TM domain) and a cytoplasmic signaling domain (also referred to herein as “an intracellular signaling domain (ICS domain)”) comprising a functional signaling domain derived from a stimulatory molecule and/or costimulatory molecule as defined below. In some aspects, the set of polypeptides are contiguous with each other. In some embodiments, the set of polypeptides include a dimerization switch that, upon the presence of a dimerization molecule, can couple the polypeptides to one another, e.g., can couple an AB domain to an ICS domain. In one aspect, the stimulatory molecule is the zeta chain associated with the T cell receptor complex. In one aspect, the cytoplasmic portion of a CAR further comprises a costimulatory domain (CS domain) comprising one or more functional signaling domains derived from at least one costimulatory molecule as defined below. In one aspect, the costimulatory molecule is chosen from the costimulatory molecules described herein, e.g., 4-1BB (i.e., CD137), DAP10 and/or CD28. In one aspect, the CAR comprises a chimeric fusion protein comprising an extracellular AB domain, a TM domain and an ICS domain comprising a functional signaling domain derived from a stimulatory molecule. In one aspect, the CAR comprises a chimeric fusion protein comprising an extracellular AB domain, a TM domain, an ICS domain comprising a functional signaling domain derived from a stimulatory molecule, and a CS domain comprising a functional signaling domain derived from a costimulatory molecule. In one aspect, the CAR comprises a chimeric fusion protein comprising an extracellular AB domain, a TM domain, an ICS domain comprising a functional signaling domain derived from a stimulatory molecule, and two CS domains each of the two comprising a functional signaling domain derived from a costimulatory molecule(s) that is/are same with or different from each other. In one aspect, the CAR comprises a chimeric fusion protein comprising an extracellular AB domain, a TM domain, an ICS domain comprising a functional signaling domain derived from a stimulatory molecule, and at least two CS domains each comprising a functional signaling domain derived from a costimulatory molecule(s) that is/are same with or different from each other. In one aspect the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane. In some embodiments, the leader sequence (LS) comprises the amino acid sequence provided in the figures. In some embodiments, the LS may be encoded by a nucleic acid sequence provided in the figures.

The term “compete”, as used herein with regard to an Ab, antigen-binding Ab fragment, of AB domain of any of the anti-TYRP1 or anti-B7H6 agents of the present invention, means that a first Ab, antigen-binding Ab fragment, or AB domain, binds to an epitope in a manner sufficiently similar to the binding of a second Ab, antigen-binding Ab fragment, or AB domain, such that the result of binding of the first Ab, antigen-binding Ab fragment, or AB domain with its cognate epitope is detectably decreased in the presence of the second Ab, antigen-binding Ab fragment, or AB domain compared to the binding of the first Ab, antigen-binding Ab fragment, or AB domain in the absence of the second Ab, antigen-binding Ab fragment, or AB domain. The alternative, where the binding of the second Ab, antigen-binding Ab fragment, or AB domain to its epitope is also detectably decreased in the presence of the first antibody, can, but need not be the case. That is, a first Ab, antigen-binding Ab fragment, or AB domain can inhibit the binding of a second Ab, antigen-binding Ab fragment, or AB domain to its epitope without that second Ab, antigen-binding Ab fragment, or AB domain inhibiting the binding of the first Ab, antigen-binding Ab fragment, or AB domain to its respective epitope. However, where each Ab, antigen-binding Ab fragment, or AB domain detectably inhibits the binding of the other Ab, antigen-binding Ab fragment, or AB domain with its cognate epitope or ligand, whether to the same, greater, or lesser extent, the two (Ab, antigen-binding Ab fragment, or AB domain) are said to “cross-compete” with each other for binding of their respective epitope(s). Both competing and cross-competing Abs, antigen-binding Ab fragments, or AB domains are encompassed by the invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing Abs, antigen-binding Ab fragments, or AB domains are encompassed and can be useful for the methods disclosed herein.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3).

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. Costimulatory molecules include, but are not limited to a protein selected from the group consisting of an MHC class I molecule, TNF receptor proteins, Immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8alpha, CD8beta, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, OX40 (CD134), 4-1BB (CD137), SLAM (SLAMF1, CD150, IPO-3), CD160 (BY55), SELPLG (CD162), DNAM1 (CD226), Ly9 (CD229), SLAMF4 (CD244, 2B4), ICOS (CD278), CEACAMI, CDS, CRTAM, DAP10, GADS, GITR, HVEM (LIGHTR), IA4, ICAM-1, IL2R beta, IL2R gamma, IL7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and a ligand that specifically binds with CD83. In embodiments wherein a CAR comprises one or more CS domain, each CS domain comprises a functional signaling domain derived from a costimulatory molecule. In some embodiments, the encoded CS domain comprises CD28, 4-1BB, or DAP10. In one embodiment, the CS domain comprises the amino acid sequence of CD28CS, 41BBCS, or DAP10CS or an equivalent.

The term “cytotoxicity” generally refers to any cytocidal activity resulting from the exposure of the immunostimulatory combination of Type 1 and Type 2 cytokines and/or anti-TYRP1 and/or anti-B7H6 agents of the invention or cells comprising the same to cells expressing TYRP1 or B7H6. This activity may be measured by known cytotoxicity assays, including IFN-γ production assays. When the target cell is a cancer or tumor cell, the term “anti-cancer cytotoxicity” or “anti-tumor cytotoxicity” may be used.

The phrase “disease associated with expression of TYRP1” or “TYRP1-associated disease” includes, but is not limited to, a disease associated with expression of TYRP1 or condition associated with cells which express TYRP1 including, e.g., proliferative diseases such as a cancer or malignancy or a precancerous condition; or a noncancer related indication associated with cells which express TYRP1. Noncancer-related indications associated with TYRP1 include fibrosis, autoimmunity, a cardiovascular condition, an allergic condition, a respiratory disease, a nephropathy, a neural disease, a muscular disease, a liver disease, metabolic syndrome, infection, and an inflammatory disorder. Examples of various cancers that express TYRP1 include but are not limited to, bladder cancer, bone cancer, brain cancer, breast cancer, carcinomas, cervical cancer, colon cancer, colorectal cancer, desmoid tumor, endometrial cancer, esophageal cancer, fibromatosis, glioblastoma, head and neck cancer, liver cancer, lung cancer, lymphoma, leukemias, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, testicular cancer, thyroid cancer, urothelial cancer, and the like.

The phrase “disease associated with expression of B7H6” or “B7H6-associated disease” includes, but is not limited to, a disease associated with expression of TYRP1 or condition associated with cells which express B7H6 including, e.g., proliferative diseases such as a cancer or malignancy or a precancerous condition; or a noncancer related indication associated with cells which express B7H6. Noncancer-related indications associated with B7H6 include fibrosis, autoimmunity, a cardiovascular condition, an allergic condition, a respiratory disease, a nephropathy, a neural disease, a muscular disease, a liver disease, metabolic syndrome, infection, and an inflammatory disorder. Examples of various cancers that express B7H6 include but are not limited to, bladder cancer, bone cancer, brain cancer, breast cancer, carcinomas, cervical cancer, colon cancer, colorectal cancer, desmoid tumor, endometrial cancer, esophageal cancer, fibromatosis, glioblastoma, head and neck cancer, liver cancer, lung cancer, lymphoma, leukemias, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, testicular cancer, thyroid cancer, urothelial cancer, and the like.

An “effective amount” or “an amount effective to treat” refers to a dose that is adequate to prevent or treat a disease, condition, or disorder in an individual. Amounts effective for a therapeutic or prophylactic use will depend on, for example, the stage and severity of the disease or disorder being treated, the age, weight, and general state of health of the patient, another pre-existing condition, and the judgment of the prescribing physician. The size of the dose will also be determined by the active ingredient selected, method of administration, timing and frequency of administration, the existence, nature, and extent of any adverse side effects that might accompany the administration of a particular active ingredient, and the desired physiological effect. It will be appreciated by one of skill in the art that various diseases or disorders could require prolonged treatment involving multiple administrations, perhaps using the inventive anti-TYRP1 or anti-B7H6 agents, nucleic acids, vectors, cells, or compositions in each or various rounds of administration.

The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences.

The term “hinge”, “spacer”, or “linker” refers to an amino acid sequence of variable length typically encoded between two or more domains or portions of a polypeptide construct to confer flexibility, improved spatial organization, proximity, etc.

As used herein, “human antibody” means an antibody having an amino acid sequence corresponding to that of an antibody produced by a human and/or which has been made using any of the techniques for making human antibodies known to those skilled in the art or disclosed herein. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hoogenboom and Winter, J. Mol. Biol., 227:381, 1991; Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, 1985; Boerner et al., J. Immunol., 147 (1):86-95, 1991; and U.S. Pat. No. 5,750,373.

The term “immune cell” refers to a cell of hematopoietic origin functionally involved in the initiation and/or execution of innate and/or adaptive immune response.

The term “intracellular signaling domain” or “ICS domain” as used herein, refers to an intracellular portion of a molecule. The intracellular signaling domain generates a signal that promotes an immune effector function of the cell transduced with a nucleic acid sequence comprising a CAR, e.g., a CAR T cell. Examples of immune effector function, e.g., in a CAR T cell, include cytolytic activity and helper activity, including the secretion of cytokines. ICS domains include an ICS domain of a lymphocyte receptor chain, a TCR/CD3 complex protein, an Fc receptor subunit, an IL-2 receptor subunit, CD3 zeta, FcR gamma, FcR beta, CD3 gamma, CD3 delta, CD3 epsilon, CD5, CD22, CD79a, CD79b, CD66d, CD278(ICOS), Fc epsilon RI, DAP10, or DAP12.

An “isolated” biological component (such as an isolated protein, nucleic acid, vector, or cell) refers to a component that has been substantially separated or purified away from its environment or other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis. An isolated nucleic acid or protein can exist in substantially purified form, or can exist in a non-native environment such as, for example, a host cell.

A “leader sequence” or “LS” as used herein, also referred to as “signal peptide,” “signal sequence,” “targeting signal,” “localization signal,” “localization sequence,” “transit peptide,” or “leader peptide” in the art, is a short peptide present at the N-terminus of the majority of newly synthesized proteins that are destined towards the secretary pathway. The core of the signal peptide may contain a long stretch of hydrophobic amino acids. The signal peptide may or may not be cleaved from the mature polypeptide.

The term “linker” as used in the context of a scFv, a CAR construct, or a cytokine construct refers to a peptide linker that consists of amino acids such as glycine and/or serine residues used alone or in combination, to link variable heavy and variable light chain regions, or other regions or domains of CAR constructs, or regions or domains of cytokine constructs together. In one embodiment, the flexible polypeptide linker is a Gly/Ser linker and comprises one or more repeats of the amino acid sequence unit GlyX-Ser-GlyY, which is also referred to as a GXSGY linker, where X is a non-zero integer and Y is zero or a non-zero integer. In one embodiment, the flexible polypeptide linker includes, but is not limited to, Gly3SerGly3, which is also referred to as G3SG3. In another embodiment, the flexible polypeptide linker includes, but is not limited to, Gly2SerGly2Ser, which is also referred to as G2SG2S. Such linkers may be encoded for example, by the nucleic acid sequences provided in the figures.

The term “mammal” refers to any mammal, including, but not limited to, mammals of the order Rodentia, such as mice, rats, and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

The term “nucleic acid” and “polynucleotide” refer to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, guide RNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism.

The term “parenteral” or “parenterally” as used herein includes any route of administration of a compound or composition, characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intradermal, intraperitoneal, intramuscular, intrasternal, intravenous, intraarterial, intrathecal, intraventricular, intraurethral, intracranial, intrasynovial injection or infusions; and kidney dialytic infusion techniques. In a preferred embodiment, parenteral administration of the compositions of the present invention comprises subcutaneous or intraperitoneal administration.

The term “pharmaceutically acceptable excipient,” “pharmaceutical excipient,” “excipient,” “pharmaceutically acceptable carrier,” “pharmaceutical carrier,” or “carrier” as used herein refers to compounds or materials conventionally used in pharmaceutical compositions during formulation and/or to permit storage.

The term “recombinant” means a polynucleotide, a protein, a cell, and so forth with semi-synthetic or synthetic origin which either does not occur in nature or is linked to another polynucleotide, a protein, a cell, and so forth in an arrangement not found in nature.

The term “scFv,” “single-chain Fv,” or “single-chain variable fragment” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL. The linker may comprise portions of the framework sequences. In scFvs, the heavy chain variable domain (HC V, HCV, or VH) may be placed upstream of the light chain variable domain (LC V, LCV, or VL), and the two domains may optionally be linked via a linker (for example, the GXSGY linker). In this case, when the scFv is for example derived from TA99, the construct may be referred to as TA99scFvHL, TA99HL, TA99scFvVHVL, or TA99VHVL. Alternatively, the heavy chain variable domain may be placed downstream of the light chain variable domain, and the two domains may optionally be linked via a linker (for example, a GXSGY linker). In this case, when the scFv is for example derived from TA99, the construct may be referred to as TA99scFvLH, TA99LH, TA99scFvVLVH, or TA99VLVH. The same naming rules apply to other similar constructs herein.

The term “signaling domain” refers to the functional portion of a protein which acts by transmitting information within the cell to regulate cellular activity via defined signaling pathways by generating second messengers or functioning as effectors by responding to such messengers.

The term “immunostimulatory cytokine,” refers to a cytokine or combination of cytokines that activates the cytoplasmic signaling sequence(s) that regulate activation of the immune cell in a stimulatory way for at least some aspect of the immune cell signaling pathway.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as an immunoreceptor tyrosine-based activation motif or ITAM. Examples of an ITAM containing cytoplasmic signaling sequence that are of particular use in the invention include, but are not limited to, those derived from CD3 zeta, common FcR gamma (FCER1G), Fc gamma RIIa, FcR beta (Fc epsilon R1b), CD3 gamma, CD3 delta, CD3 epsilon, CD79a, CD79b, DAP10, and DAP12. In a specific CAR of the invention, the intracellular signaling domain in any one or more CARs of the invention comprises an intracellular signaling sequence, e.g., a primary signaling sequence of CD3 zeta. In a specific CAR of the invention, the primary signaling sequence of human CD3 zeta, referred to as “CD3zICS” herein, is the amino acid sequence provided in the figures and may be encoded by the nucleotide sequence provided in the figures. Alternatively, equivalent residues from a non-human or mouse species, e.g., rodent, monkey, ape and the like, may be utilized.

The term “subject” as used herein may be any living organisms, preferably a mammal. In some embodiments, the subject is a primate such as a human. In some embodiments, the primate is a monkey or an ape. The subject can be male or female and can be any suitable age, including infant, juvenile, adolescent, adult, and geriatric subjects. In some examples, the patient or subject is a validated animal model for disease and/or for assessing toxic outcomes. The subject may also be referred to as “patient” in the art. The subject may have a disease or may be healthy.

The term “synthetic Ab” or “synthetic antigen-binding Ab fragment” as used herein, refers to an Ab or antigen-binding Ab fragment which is generated using recombinant DNA technology, such as, for example, an antibody expressed by a yeast as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “target” as used herein refers to the molecule that an anti-TYRP1 or anti-B7H6 agents of the present invention specifically binds to. The term also encompasses cells and tissues expressing the target molecule and also diseases that are associated with expression of the target.

The term “target cell” as used herein refers to a cell expressing the target molecule (such as TYRP1 or B7H6) of the anti-TYRP1 or anti-B7H6 agents of the present invention on the cell surface. In some embodiments, the target cell is a cancer cell or tumor cell. In some embodiments, the target cell is a vascular cell. In some embodiments, the target cell is a fibroblast. In some embodiments, the target cell is an epithelial cell. In some embodiments, the target cell is a cell type that has a particular role in the pathology of cancer or inflammation. In some embodiments, the target cell is a cell type that has a particular role in the pathology of a disease such as but not limited to cancer, fibrosis, autoimmunity, an inflammatory disease, a cardiovascular condition, a metabolic disease, an allergic condition, a respiratory disease, a nephropathy, a neural disease, a muscular disease, a liver disease, metabolic syndrome, infection, and an inflammatory disorder.

The term “target molecule” as used herein refers to a molecule that is targeted by the anti-TYRP1 or anti-B7H6 agents of the present invention. The AB domain of the anti-TYRP1 or anti-B7H6 agents of the present invention has a binding affinity for the target molecule. In some embodiments, the target molecule is TYRP1. In some embodiments, the target molecule is B7H6.

The term “transfected,” “transformed,” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

By the term “transmembrane domain” or “TM domain”, what is implied is any three-dimensional protein structure which is thermodynamically stable in a membrane. This may be a single alpha helix, a transmembrane beta barrel, a beta-helix of gramicidin A, or any other structure. Transmembrane helices are usually about 20 amino acids in length. Typically, the transmembrane domain denotes a single transmembrane alpha helix of a transmembrane protein, also known as an integral protein.

As used herein, the term “treat,” “treatment,” or “treating” generally refers to the clinical procedure for reducing or ameliorating the progression, severity, and/or duration of a disease or of a condition, or for ameliorating one or more conditions or symptoms (preferably, one or more discernible ones) of a disease. The type of disease or condition to be treated may be, for example, but are not limited to, cancer, fibrosis or fibrotic disease, autoimmunity, a cardiovascular condition, an allergic condition, a respiratory disease, a nephropathy, a neural disease, a muscular disease, a liver disease, metabolic syndrome, infection, an inflammatory disorder, and an infectious disease. Examples of cancer include, but are not limited to, bladder cancer, bone cancer, brain cancer, breast cancer, carcinomas, cervical cancer, colon cancer, colorectal cancer, desmoid tumor, endometrial cancer, esophageal cancer, fibromatosis, glioblastoma, head and neck cancer, liver cancer, lung cancer, lymphoma, leukemias, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, testicular cancer, thyroid cancer, urothelial cancer, or a combination of any of the foregoing. Examples of an infectious disease include, but are not limited to, viral, bacterial, yeast, fungal, or parasite. In specific embodiments, the effect of the “treatment” may be evaluated by the amelioration of at least one measurable physical parameter of a disease, resulting from the administration of one or more therapies (e.g., an immunostimulatory combination of super-2 and IL-33 cytokines administered by an anti-TYRP1 CAR expressing cell). The parameter may be, for example, gene expression profiles, the mass of disease-affected tissues, inflammation-associated markers, cancer-associated markers, the number or frequency of disease-associated cells, tumor burden, the presence or absence of certain cytokines or chemokines or other disease-associated molecules, and may not necessarily discernible by the patient. In other embodiments “treat”, “treatment,” or “treating” may result in the inhibition of the progression of a disease, either physically by, e.g., stabilization of a discernible symptom, physiologically by, e.g., stabilization of a physical parameter, or both. In other embodiments the terms “treat”, “treatment” and “treating” refer to the reduction or stabilization of cancerous tissue or cells. Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment or prevention effects of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof. Having provided definitions, the invention is now further described.

The invention is particularly directed to novel immunostimulatory combinations of cytokines, administered either as soluble proteins or expressed by a recombinant or isolated cell which further expresses a chimeric antigen receptor (CAR). In particular, the invention comprises a combination of a Type 1 cytokine which comprises IL-2, e.g., human IL-2 or an IL-2 like cytokine or mutation of human IL-2 such as Super-2 and a Type 2 cytokine, e.g., TSLP, IL-25, IL-33, IL-4 (IL4), IL-5 (IL5), IL-6 (IL6), IL-10 (IL10), and IL-13 (IL13), and preferably IL-33, IL-25 or TSLP.

A particularly preferred immunostimulatory cytokine combination according to the invention comprises human IL-2 or Super-2 and IL-33. As mentioned, such cytokine combination may be administered as soluble proteins or either or both of these cytokines may be administered on recombinant cells engineered to express either or both of the IL-2-like cytokine and the type-2 cytokine. Such recombinant cells may include any cell suitable for in vivo administration. In exemplary embodiments such recombinant cells will endogenously express or will be engineered to express an endogenous or chimeric receptor that recognizes a target antigen or ligand expressed on target cells, e.g., tumor or infected cells and either or both of the IL-2-like cytokine and the type-2 cytokine. Such recombinant cells may include T, NK or other immune or non-immune cells which express an endogenous or chimeric receptor that recognizes an antigen or ligand expressed on target cell. In exemplary embodiments the cells will comprise CAR-T cells which express a chimeric receptor that recognizes an antigen or ligand expressed on target cells, typically cancer or infected cells, and most typically solid tumor cells, and which further express either or both of the IL-2-like cytokine and the type-2 cytokine, e.g., IL-33, IL-25 or TSLP.

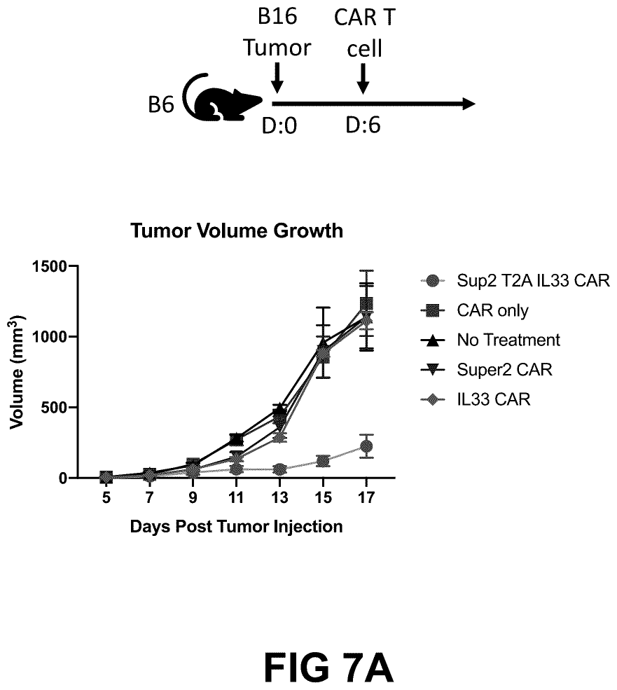

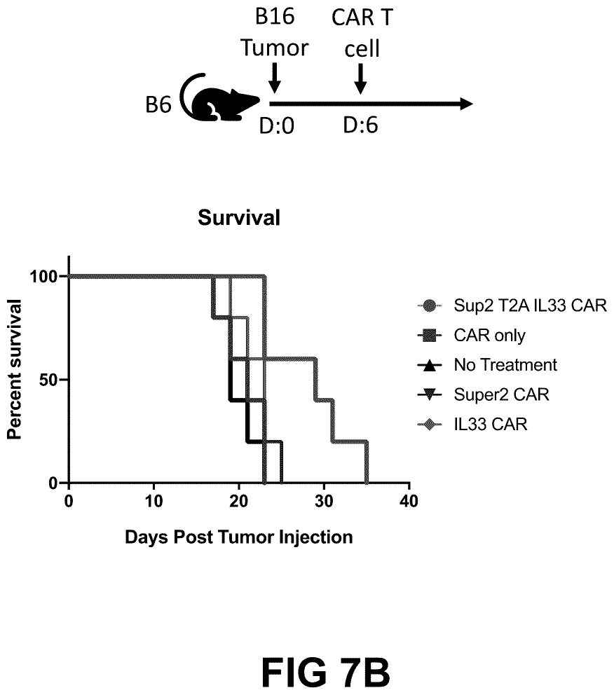

As noted, a preferred application of the subject cytokine combination is in the treatment of solid tumors. In particular, as shown herein, administration of the disclosed immunostimulatory combination comprising IL-2 or an IL-2 like cytokine such as Super2 and said Type 2 cytokine, such as IL-33, has been demonstrated to elicit a synergistic or additive effect on antitumor immunity when expressed by CAR T cells, particularly antitumor responses against solid tumors such as B16F10 melanomas as shown in FIG. 7, compared to either type 1 or Type 2 cytokine which, when administered singly, were ineffective at eliciting an immune response to inhibit tumor growth rates.

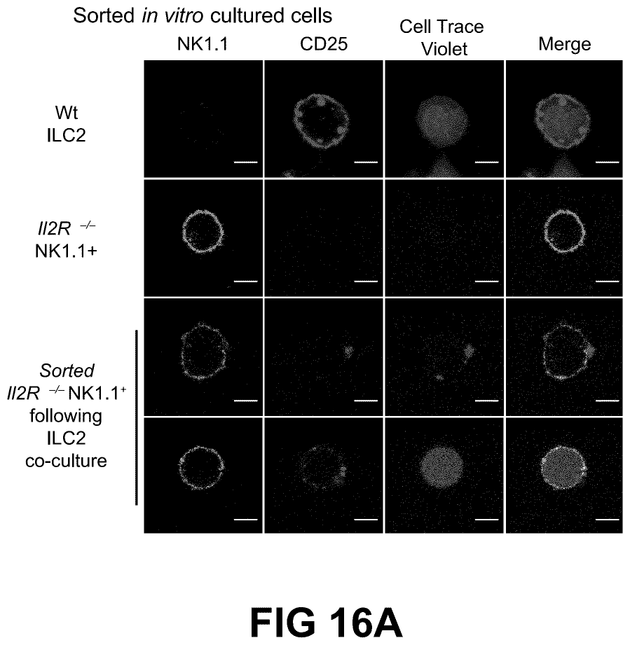

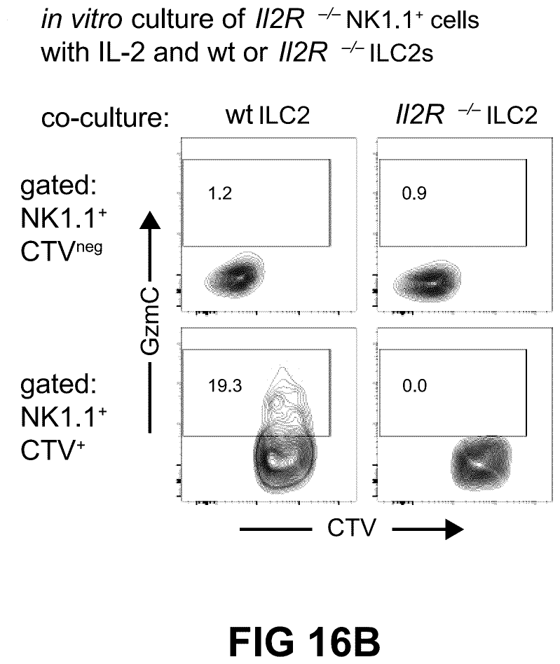

While not wishing to be bound by theory, it is believed that such additive or synergistic effects are obtained because Type II cytokines, such as IL-33 and IL-25 and also TSLP, when administered in combination with IL-2 like cytokines activate ILC (innate lymphoid cells), which act as helper cells to activate and expand CD8 T cells and NK cells. This possible mechanism for synergism is further supported by data, as shown herein, indicating that Super2 and IL-33 act on 2 different cell types. IL-33 stimulates type 2 innate lymphoid cells (ILC2) while Super2 activates cells that express CD122 (NK cells and T cells). As shown in FIG. 16A, ILC2 cells act as helpers that transfer CD25 (red) to NK cells (green) and presumably T cells, resulting in improved expression of effector granzymes as shown in FIG. 16B.

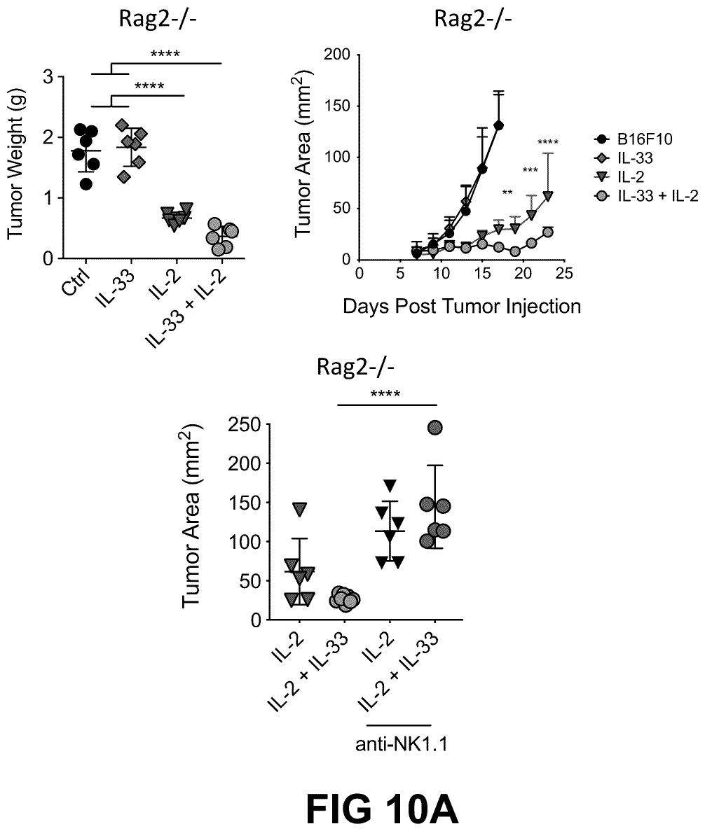

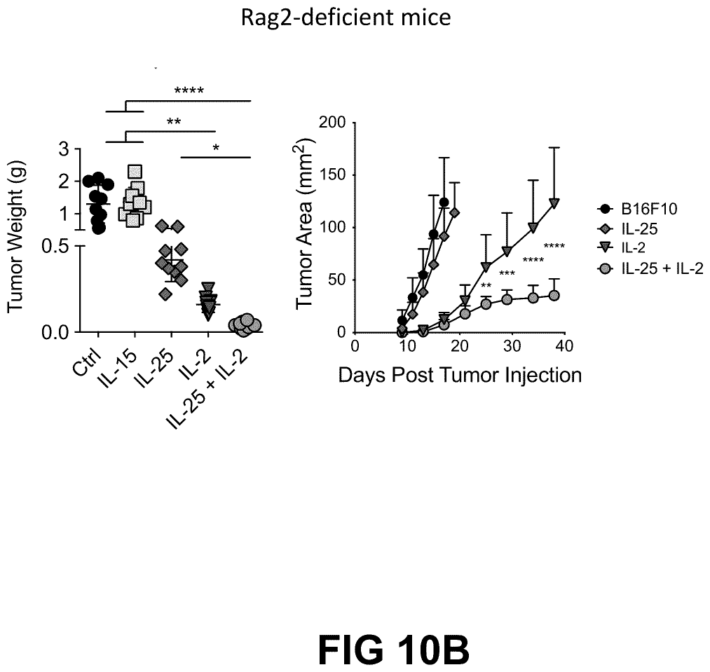

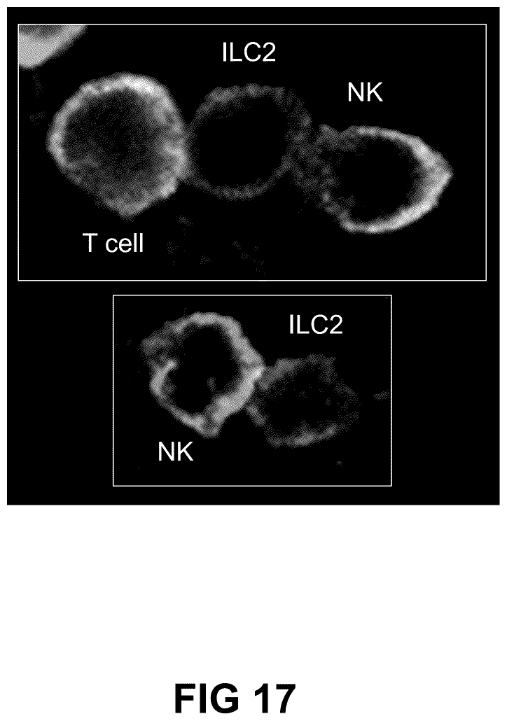

Synergistic effects seem to be dependent on cell-cell interactions between NK cells or T cells and type 2 innate lymphoid cells (ILC2), which are activated by Type-2 cytokines. Immunofluorescent imaging of B16F10 melanoma tumor sections show direct interactions of ILC2 cells with T cells and NK cells as show in FIG. 17. Further indirect evidence of this theory includes experimental results in Rag2 KO mice, wherein B cell and T cell development is arrested, leaving immune responses to be mediated by other cells, such as NK cells. Such Rag2 KO mice were challenged with B16F10 melanoma model as shown in FIG. 10. Expression of Type 1 cytokine (IL-2) marginally delayed tumor growth and expression of Type 2 cytokine (IL-33) had little to no effect. In contrast, expression of the IL-2/IL-33 combination delayed tumor growth to a greater extent than either cytokine alone. These effects were dramatically reduced by addition of and NK1.1, an antibody which prevents essential cell-cell interactions, indicating that NK cells are essential for synergistic antitumor effects.

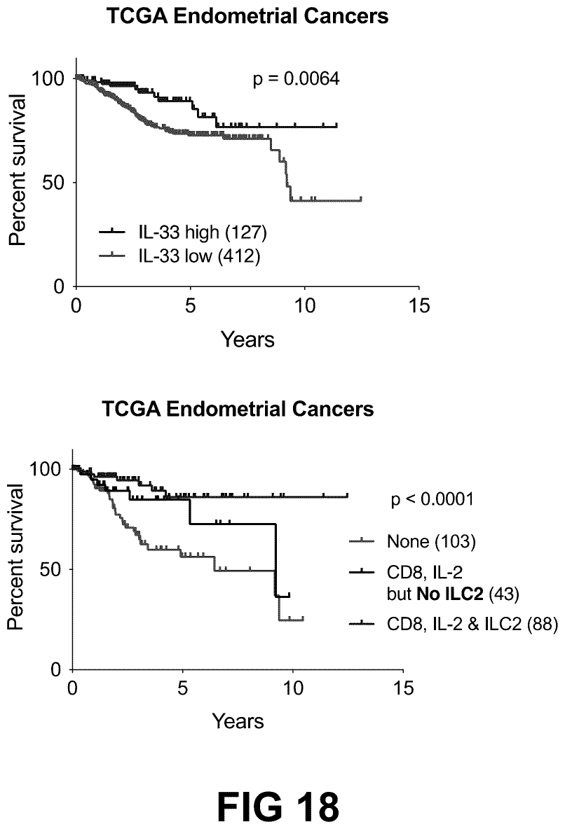

Further indirect supporting evidence is the observation that in the case of endometrial cancer (where there is a substantial need for effective treatment, particularly for high grade cancers), patients expressing higher IL-33 levels, or alternatively co-expression of IL-2 in association with ILC2 and CD8 T cell-associated genes, is correlated with better prognosis as shown in FIG. 18. This correlation suggests a cooperation between Type I and Type II immune responses. Further evidence that these cytokines elicit unexpectedly better results in combination is the inventors' observation that Introducing some cytokines alone, e.g., IL-15, elicited adverse effects on antitumor immunity (IL-15 and IL-15R needed to be co-expressed, otherwise IL-15 competes with IL-2 for receptor binding) as shown in FIG. 10B.

Further supporting evidence for synergistic effects of Type 1 and Type 2 cytokine combinations are seen in another embodiment of the present invention, wherein the Type 1 cytokine (IL-2) combined with Type 2 cytokine (IL-25) elicited a synergistic or additive effect on antitumor immunity when expressed in C57BL/6 mice bearing B16F10 melanoma tumors, compared to either IL-2 or IL-25 acting alone, as shown in FIG. 10B. In a similar manner to IL-33, the Type 2 cytokine, IL-25, also stimulates ILC2 cells to act as helpers for activation and recruitment of CD8 T and NK cells, therefore a similar mechanism of unexpected synergistic antitumor immunity of, as described above for the combination of IL-2/IL-33, is implied.

Delivery of the disclosed combination of immunostimulatory cytokines activates the endogenous immune response leading to growth inhibition of solid and/or disseminated tumors. In some instances, delivery of the disclosed immunostimulatory combinations of recombinant cytokines as soluble proteins or fragments thereof is sufficient to control or inhibit tumor growth. In other instances, delivery of one or both cytokine to the tumor microenvironment by means of an engineered immune cell expressing a CAR, which binds to an antigen expressed within said microenvironment, is sufficient to inhibit tumor growth.

The invention provides for administration of immunostimulatory Type 1 and Type 2 cytokines, administered either separately or in combination. In general, Type 1 cytokines are secreted signaling proteins that bind to and activate a wide range of immune cells, thereby favoring the development of a strong cellular immune response. In some embodiments, the Type 1 cytokine is expressed as the full length or truncated protein. In some embodiments, the Type 1 cytokine is expressed as a fusion construct wherein the Type 1 cytokine is tethered to a second cytokine by a self-cleaving peptide. In general, Type 2 cytokines are secreted signaling proteins that drive the production of T helper-2 (Th2) cells, thereby favoring a strong humoral, antibody-mediated immune response. In some embodiments, the Type 2 cytokine is expressed as the full length or truncated protein. In some embodiments, the Type 2 cytokine is expressed as a fusion construct wherein the Type 2 cytokine is linked to a second cytokine by a self-cleaving peptide. The inventive cytokine variations can be adapted to the tumor microenvironment within the individual patient to elicit synergistic or additive antitumor effects.

The immunostimulatory cytokine combinations may be expressed as soluble recombinant proteins or by a recombinant or isolated cell under constitutive or inducible conditions. In some embodiments, both said Type 1 and Type 2 cytokines are expressed separately as soluble recombinant proteins which optionally are modified to improve in vivo half-life. Either or both of said Type 1 and Type 2 cytokines may be expressed by a recombinant or isolated cell. In some embodiments, either said Type 1 cytokine or said Type 2 cytokine is expressed as a soluble protein and the other cytokine is expressed by a recombinant or isolated cell engineered to express said Type 1 or Type 2 cytokine. Such variation of expression allows for variation of cytokine administration, which further allows for tailoring therapy to elicit a synergistic antitumor immune response within an individual patient.

In some embodiments, either said Type 1 cytokine and/or said Type 2 cytokine is expressed by a recombinant or isolated cell engineered to express said Type 1 and/or Type 2 cytokine and a CAR. In some embodiments, the CAR binds specific target molecules within the tumor, thereby localizing expression of the inventive immunostimulatory cytokine combinations to the tumor microenvironment. In some embodiments, the cell is activated or stimulated to proliferate when the CAR binds to its target molecule. In some embodiments, the cell is activated or stimulated to release or secrete said type 1 and/or said type 2 cytokine when the CAR binds to its target molecule. In some embodiments, the cell exhibits cytotoxicity against tumor cells expressing the target molecule when the CAR binds to the target molecule. In some embodiments, administration of the cell alone or in combination with a type 1 or type 2 cytokine results in enhanced killing of target cells because of the additive or synergistic effects of the combination of the type 1 and type 2 cytokine on immunity, optionally antitumor immunity. These combined effects of the functional features of the inventive cell engineered to express both Type 1 and/or Type 2 cytokines and a CAR result in improved efficacy for the treatment of disseminated and solid cancers.

Immunostimulatory Cytokine Combinations

An exemplary cytokine construct of the invention may comprise at least one Type 1 cytokine, selected from the group consisting of interleukin-2 (IL-2), an IL-2 superkine variant (super-2), mouse serum albumin fused to IL-2 (MSA/IL-2), and interleukin-21 (IL-21). In a preferred embodiment, a Type 1 cytokine construct may contain MSA/IL-2.

In some embodiments, a cytokine construct of the invention may comprise at least one Type 2 cytokine, selected from the group consisting of interleukin-4 (IL-4), interleukin-5 (IL-5), interleukin-25 (IL-25), interleukin-33 (IL-33), and thymic stromal lymphopoietin (TSLP). In another preferred embodiment, a Type 1 cytokine construct may contain IL-25.

In some embodiments, a construct comprising a Type-1 cytokine may be tethered to a Type-2 cytokine by a self-cleaving peptide linker. In a preferred embodiment, a Type 1 cytokine is IL-2, a Type 2 cytokine is IL-33, and a self-cleaving peptide linker is T2A.

The present invention in general relates to an immunostimulatory cytokine combination comprising a type 1 cytokine comprising an IL-2 like cytokine, e.g., human IL-2 or Super-2 or IL-21 and a type 2 cytokine, compositions and/or recombinant cells engineered to express same and the use of the foregoing as a therapeutic, e.g., to treat cancer or an infectious condition.

In some exemplary embodiments the invention provides a method of treating cancer or infection, the method comprising administering to a subject in need thereof an immunostimulatory cytokine combination comprising a prophylactically therapeutically effective amount of
(a) at least one Type I cytokine which comprises an interleukin-2-like (IL-2-like) or IL-21 cytokine, and
(b) at least one Type II cytokine, optionally IL-33, wherein said at least one cytokine (a) and (b) are administered in the same or different compositions, and preferably at dosages wherein said at least one cytokine (a) and (b) in combination elicit a synergistic or additive effect on immunity compared to cytokine (a) or cytokine (b) alone.

In some exemplary embodiments the administered Type 1 cytokine is selected from IL-2 (IL2), desleukin (Proleukin), Interking (recombinant IL-2 with a serine at residue 125), Neoleukin 2/15, Super-2, other IL-2 variants and IL-21 and the Type 2 cytokine is selected from TSLP, IL-25, IL-33, IL-1(IL1), IL-4 (IL4), IL-5 (IL5), IL-6 (IL6), IL-10 (IL10)4 (IL4), and IL-13 (IL13).

In some exemplary embodiments the administered immunostimulatory combination comprises Superkine IL-2 (Super-2) and IL-33 or IL-25 or TSLP.

In some exemplary embodiments the administered immunostimulatory combination is used to treat at least one cancer.

In some exemplary embodiments the administered immunostimulatory combination is used to treat at least one solid tumor, optionally wherein the solid tumor is selected from bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, thyroid cancer or a combination of any of the foregoing.

In some exemplary embodiments the administered immunostimulatory combination additively or synergistically activates the endogenous immune response providing for enhanced inhibition of tumor growth, migration and/or metastasis.

In some exemplary embodiments either or both of said cytokines are administered in soluble form.

In some exemplary embodiments either or both of said administered cytokines are expressed by a recombinant cell, e.g., an immune cell.

In some exemplary embodiments either or both of said administered cytokines are expressed by a recombinant cell, e.g., a cell which expresses an endogenous or chimeric receptor or binding domain, e.g., an antibody binding domain, which binds to a ligand or receptor expressed by target cells, e.g., cancer or infected cells.

In some exemplary embodiments either or both of said administered cytokines are expressed by a CAR expressing cell, e.g., a CAR-T or CAR-NK cell.

In some exemplary embodiments either or both of said administered cytokines are expressed by cells which express either or both of Super-2 and IL-33 and further express a receptor or binding domain that binds to an antigen or ligand expressed on target cells, e.g., a natural or synthetic or chimeric receptor or an antibody or antibody fragment that binds to target (e.g., tumor) cells.

In some exemplary embodiments the Type I and Type II cytokines are administered separately.

In some exemplary embodiments said at least one Type I and Type II cytokines are administered in combination.

In some exemplary embodiments either or both of the Type I and Type II cytokines are administered as a soluble protein, e.g., via injection, optionally by intraperitoneal administration, intravenous infusion, intramuscularly, intratumorally, subcutaneously; sublingually, intranasally, or other conventional route of administration of soluble proteins.

In some exemplary embodiments said at least one Type I and Type II cytokine in combination elicit a synergistic effect on immunity, optionally on antitumor immunity.

In some exemplary embodiments either or both of said Type I and Type II cytokines are expressed by a recombinant or isolated cell which expresses either or both of said Type I and Type 11 cytokines under constitutive or inducible conditions.

In some exemplary embodiments said recombinant or isolated cell optionally comprises an immune cell, further optionally selected from the group consisting of a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH17 cell, a TH9 cell, a TH22 cell, a follicular helper T cells, an a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, and a dendritic cell, preferably a CAR-T cell.

In some exemplary embodiments the recombinant or isolated cell expresses at least one natural or chimeric antigen receptor (CAR) which binds to an antigen expressed by target cells, optionally tumor, infected or other diseased cells.

In some exemplary embodiments the recombinant cell expresses at least one chimeric antigen receptor (CAR) comprises:
(a) an antigen-binding (AB) domain that binds to a target molecule which is expressed on the surface of a tumor, a virus, or virus infected cell in a patient or which is overexpressed or aberrantly expressed in cancer, viral infection, or other disease associated with expression of the antigen bound by the AB domain,
(b) a transmembrane (TM) domain,
(c) an intracellular signaling (ICS) domain,
(d) optionally a hinge that joins said AB domain and said TM domain, and
(e) optionally one or more additional costimulatory (CS) domains.

In some exemplary embodiments the administered recombinant or isolated cell comprises at least one nucleic acid encoding a CAR and at least one nucleic acid encoding said Type I cytokine and/or said Type II cytokine.

In some exemplary embodiments:
(viii) the cell is activated or stimulated to proliferate when the CAR binds to its target molecule;
(ix) the cell is activated or stimulated to release or secrete said type 1 and/or said type II cytokine when the CAR binds to its target molecule;
(x) the cell exhibits cytotoxicity against cells expressing the target molecule when the CAR binds to the target molecule;
(xi) administration of the cell ameliorates a disease, e.g., an infectious disease or a neoplastic disease, e.g., a solid tumor associated condition, when the CAR binds to its target molecule; and/or
(xii) administration of the cell alone or in combination with a type I or type II cytokine results in enhanced killing of target cells because of the additive or synergistic effects of the combination of the type I and type II cytokine on immunity, optionally antitumor immunity.

In some exemplary embodiments:
(d) both Type I and Type II cytokines are administered as soluble proteins,
(e) both Type I and Type II cytokines are expressed by a recombinant or isolated cell, e.g., a CAR-T cell engineered to express said Type I and Type II cytokines, or
(f) either the type I cytokine or the type II cytokine is administered as a soluble protein and the other cytokine is expressed by a recombinant or isolated cell, e.g., a CAR-T cell engineered to express said Type I or Type II cytokine.

In some exemplary embodiments the immunostimulatory combination is used for stimulating an immune response in a subject in need thereof, optionally a synergistic immune response, further optionally a synergistic antitumor immune response, preferably against a solid tumor, optionally wherein the cancer is selected from the group consisting of bladder cancer, bone cancer, brain cancer, breast cancer, colon cancer, colorectal cancer, desmoid tumor, endometrial cancer, esophageal cancer, fibromatosis, glioblastoma, head and neck cancer, liver cancer, lung cancer, e.g., non-small cell lung cancer or small cell lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, thyroid cancer or a combination of any of the foregoing.

In some exemplary embodiments the immunostimulatory combination is used for the treatment of infectious disease, wherein the infection is selected from the group consisting of viral, bacterial, yeast, fungal, or parasite.

In some exemplary embodiments the invention provides a composition comprising an immunostimulatory combination comprising at least one immune stimulating Type I cytokine, optionally selected from the group consisting of interleukin-21, interleukin-2 (IL-2), and an IL-2 superkine variant (super-2); preferably super-2 and a Type 11 cytokine optionally selected from the group consisting of interleukin-4 (IL-4), interleukin-5 (IL-5), interleukin-25 (IL-25), interleukin-33 (IL-33), and Thymic stromal lymphopoietin (TSLP), or an immunologically active fragment, variant or fusion protein comprising any of the foregoing, preferably IL-33 or an IL-33 fusion protein, which Type I and Type 11 cytokine when administered in combination elicit an additive or synergistic effect on immunity.

In some exemplary embodiments the invention provides a composition comprising an immunostimulatory combination as above described wherein the Type I cytokine comprises or consists of an amino acid sequence at least 80%, at least 85%, at least 90%, at least 92%, at least 94%, at least 95%, at least 96%, at least 97%, at least 98%, at least 99%, or 100% identical to human IL-2 or Super-2.

In some exemplary embodiments the invention provides a composition comprising an immunostimulatory combination as above described wherein the Type 11 cytokine is selected from the group consisting of interleukin-4 (IL-4), interleukin-5 (IL-5), interleukin-25 (IL-25), interleukin-33 (IL-33), and Thymic stromal lymphopoietin (TSLP) and immunologically active fragments, variants and fusion proteins comprising any of the foregoing.

In some exemplary embodiments the invention provides a composition comprising an immunostimulatory combination as above described wherein the Type 11 cytokine has an amino acid sequence at least 80%, at least 85%, at least 90%, at least 92%, at least 94%, at least 95%, at least 96%, at least 97%, at least 98%, at least 99%, or 100% identical to that of IL-4, IL-5, IL-25, IL-33, or TSLP.

In some exemplary embodiments the invention provides an isolated or recombinant cell which is engineered to express at least one Type I cytokine which comprises an IL-2 like cytokine, optionally selected from the group consisting of interleukin-2 (IL-2), further optionally human IL-2, an IL-2 superkine variant (super-2) or other IL-2 variant; and at least one Type II cytokine, optionally selected from the group consisting of interleukin-4 (IL-4), interleukin-5 (IL-5), interleukin-25 (IL-25), interleukin-33 (IL-33), and Thymic stromal lymphopoietin (TSLP), preferably IL-33 or an IL-33 fusion protein, which cytokines when expressed in combination elicit an additive or synergistic effect on immunity, optionally antitumor immunity, further optionally an immune cell, optionally selected from the group consisting of a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH17 cell, a TH9 cell, a TH22 cell, a follicular helper T cells, an a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, and a dendritic cell.

In some exemplary embodiments the invention provides a recombinant cell as above described, which further expresses a chimeric antigen receptor (CAR) comprising:
(a) an antigen-binding (AB) domain that binds to a target molecule which is expressed on the surface of a tumor, a virus, or virus infected cell or which is overexpressed or aberrantly expressed in cancer or viral infection, or other disease,
(b) a transmembrane (TM) domain,
(c) an intracellular signaling (ICS) domain,
(d) optionally a hinge that joins said AB domain and said TM domain, and
(e) optionally one or more costimulatory (CS) domains.

In some exemplary embodiments the invention provides a recombinant cell expressing a CAR as above described, wherein the CAR binds to a target molecule selected from an antigen, ligand or receptor expressed by tumor or infected cells, optionally a Tyrosinase-related protein 1 (TYRP1), or a stress-inducible natural killer (NK) cell ligand (B7H6 et al.).

In some exemplary embodiments the invention provides a recombinant cell expressing a CAR as above described, wherein the AB domain in the CAR comprises an antibody (Ab) or an antigen-binding fragment thereof that binds to said target molecule, wherein said Ab or antigen-binding fragment thereof is optionally selected from a group consisting of a monoclonal Ab, a monospecific Ab, a polyspecific Ab, a humanized Ab, a tetrameric Ab, a tetravalent Ab, a multispecific Ab, a single chain Ab, a domain-specific Ab, a single-domain Ab (dAb), a domain-deleted Ab, an scFc fusion protein, a chimeric Ab, a synthetic Ab, a recombinant Ab, a hybrid Ab, a mutated Ab, CDR-grafted Ab, a fragment antigen-binding (Fab), an F(ab′)2, an Fab′ fragment, a variable fragment (Fv), a single-chain Fv (scFv) fragment, an Fd fragment, a dAb fragment, a diabody, a nanobody, a bivalent nanobody, a shark variable IgNAR domain, a VHH Ab, a camelid Ab, and a minibody.

In some exemplary embodiments the invention provides a recombinant cell expressing a CAR as above described, wherein the TM domain in the CAR is derived from the TM region, or a membrane-spanning portion thereof, of a protein selected from the group consisting of CD28, CD3e, CD4, CD5, CD8, CD9, CD16, CD22, CD33, CD37, CD45, CD64, CD80, CD86, CD134, CD137, CD154, TCRa, TCRb, and CD3z.

In some exemplary embodiments the invention provides a recombinant cell expressing a CAR as above described, wherein the CAR comprises a TM domain is derived from the TM region of CD28, or a membrane-spanning portion thereof.

In some exemplary embodiments the invention provides a recombinant cell expressing a CAR as above described, wherein the CAR comprises an ICS domain derived from a cytoplasmic signaling sequence, or a functional fragment thereof, of a protein selected from the group consisting of CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, and DAP12.

In some exemplary embodiments the invention provides a recombinant cell expressing a CAR as above described, wherein the CAR comprises an ICS domain derived from a cytoplasmic signaling sequence of CD3z, or a functional fragment thereof.

In some exemplary embodiments the invention provides a recombinant cell expressing a CAR as above described, wherein the CAR comprises a hinge derived from CD28 or other costimulatory protein.

In some exemplary embodiments the invention provides a recombinant cell expressing a CAR as above described, wherein the CAR comprises one or more CS domains if present is/are derived from a cytoplasmic signaling sequence, or functional fragment thereof, of a protein selected from the group consisting of CD28, DAP10, 4-1BB (CD137), CD2, CD4, CD5, CD7, CD8α, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAMI, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and CD83 ligand.

In some exemplary embodiments the invention provides a nucleic acid construct which comprises a nucleic acid that encodes for a type I cytokine, optionally selected from the group consisting of interleukin-21 (IL-21), interleukin-2 (IL-2), and an IL-2 superkine variant (super-2); preferably super-2, and a nucleic acid that encodes for a type II cytokine, optionally selected from the group consisting of interleukin-4 (IL-4), interleukin-5 (IL-5), interleukin-25 (IL-25), interleukin-33 (IL-33), and Thymic stromal lymphopoietin (TSLP), preferably IL-33, which Type I and Type II cytokine when expressed in combination elicit an additive or synergistic effect on immunity.

In some exemplary embodiments the invention provides a nucleic acid construct as above-described, which further encodes a natural receptor or a CAR which binds to target cells, wherein the CAR optionally comprises
(a) an AB domain or receptor that binds to a ligand, antigen or receptor expressed by target cells, e.g., tumor or infected cells,
(b) a transmembrane (TM) domain,
(c) an intracellular signaling (ICS) domain,
(d) optionally a hinge that joins said AB domain and said TM domain, and
(e) optionally one or more costimulatory (CS) domains.

In some exemplary embodiments the invention provides a nucleic acid construct as above-described, which encodes an Ab or Ab fragment, wherein the Ab or Ab fragment binds to an antigen expressed on tumor cells, e.g., bladder cancer, bone cancer, brain cancer, breast cancer, colon cancer, colorectal cancer, desmoid tumor, endometrial cancer, esophageal cancer, fibromatosis, glioblastoma, head and neck cancer, liver cancer, lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, or thyroid cancer cells or a combination of any of the foregoing.

In some exemplary embodiments the invention provides a nucleic acid construct as above-described, wherein the CAR comprises a TM domain derived from the TM region, or a membrane-spanning portion thereof, of a protein selected from the group consisting of CD28, CD3e, CD4, CD5, CD8, CD9, CD16, CD22, CD33, CD37, CD45, CD64, CD80, CD86, CD134, CD137, CD154, TCRa, TCRb, and CD3z.

In some exemplary embodiments the invention provides a nucleic acid construct as above-described, wherein the CAR comprises a TM domain derived from the TM region of CD28, or a membrane-spanning portion thereof.

In some exemplary embodiments the invention provides a nucleic acid construct as above-described, which wherein the CAR comprises a ICS domain derived from a cytoplasmic signaling sequence, or a functional fragment thereof, of a protein selected from the group consisting of CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, and DAP12.

In some exemplary embodiments the invention provides a nucleic acid construct as above-described, which the CAR comprises at least one or more CS domains is derived from a cytoplasmic signaling sequence, or functional fragment thereof, of a protein selected from the group consisting of CD28, DAP10, 4-1BB (CD137), CD2, CD4, CD5, CD7, CD8α, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAMI, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and CD83 ligand.

In some exemplary embodiments the invention provides vectors comprising any of the afore-described nucleic acids.

In some exemplary embodiments the vector is selected from a DNA, an RNA, a plasmid, a cosmid, a viral vector, a lentiviral vector, an adenoviral vector, or a retroviral vector.

In some exemplary embodiments the invention provides recombinant or isolated cells comprising at least one nucleic acid or vector as above-described.

In some exemplary embodiments the recombinant or isolated cell as above-described is a non-mammalian cell, optionally selected from the group consisting of a plant cell, a bacterial cell, a fungal cell, a yeast cell, a protozoa cell, and an insect cell.

In some exemplary embodiments the recombinant or isolated cell as above-described is a mammalian cell, optionally selected from the group consisting of a human cell, a monkey cell, a rat cell, and a mouse cell.

In some exemplary embodiments the recombinant or isolated cell as above-described is a stem cell.

In some exemplary embodiments the recombinant or isolated cell as above-described is a primary cell, optionally a human primary cell or derived therefrom.

In some exemplary embodiments the recombinant or isolated cell is a cell line, optionally a hybridoma cell line.

In some exemplary embodiments the recombinant or isolated cell as above-described is an immune cell.

In some exemplary embodiments the recombinant or isolated cell as above-described is MHC+ or MHC−.

In some exemplary embodiments the recombinant or isolated cell as above-described is selected from the group consisting of a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH17 cell, a TH9 cell, a TH22 cell, a follicular helper T cells, an a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, and a dendritic cell.

In some exemplary embodiments the recombinant or isolated cell as above-described is a T cell or T cell progenitor cell.

In some exemplary embodiments the recombinant or isolated cell as above-described is a T cell which has been modified such that its endogenous T cell receptor (TCR) is
(i) not expressed,
(ii) not functionally expressed, or
(iii) expressed at reduced levels compared to a wild-type T cell.

In some exemplary embodiments the recombinant or isolated cell as above-described is activated or stimulated to proliferate and/or to release at least one cytokine, and/or to increase expression of cytokines and/or chemokines and/or to elicit killing of target cells when it binds to a target molecule.

In some exemplary embodiments the administration of the cell as above-described ameliorates a disease, preferably cancer or infectious disease, optionally a solid tumor.

In some exemplary embodiments the invention provides a population of cells comprising at least one recombinant or isolated cell as above-described.

In some exemplary embodiments the invention provides a pharmaceutical composition comprising (a) a cytokine combination and/or at least one recombinant cell according to any of the foregoing claims and (b) a pharmaceutically acceptable excipient or carrier.

### Vectors

The present invention also provides vectors in which a polynucleotide encoding an immunostimulatory combination of Type 1 and Type 2 cytokines and the CARs of the present invention is inserted.

The vector may be, for example, a DNA vector or a RNA vector. The vector may be, for example, but not limited to, a plasmid, a cosmid, or a viral vector. The viral vector may be a vector of a DNA virus, which may be an adenovirus, or an RNA virus, which may be a retrovirus. Types of vectors suitable for Abs, antigen-binding Ab fragments, and/or CARs are well known in the art (for example, see Rita Costa A. et al., Eur J Pharm Biopharm. 2010 February; 74(2):127-38. doi: 10.1016/j.ejpb.2009.10.002. Epub 2009 Oct. 22; Frenzel A. et al. Front Immunol. 2013; 4: 217. Published online 2013 Jul. 29. doi: 10.3389/fimmu.2013.00217).

When the host cells are insect cells, such as for producing Abs or antigen-binding Ab fragments, insect-specific viruses may be used. Examples of the insect-specific viruses include, but are not limited to, the family of Baculoviridae, particularly the Autographa californica nuclear polyhedrosis virus (AcNPV). When the host cells are plant cells, plant-specific viruses and bacteria, such as Agrobacterium tumefaciens, may be used.

For expressing vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. This would be particularly beneficial for expressing CAR constructs.

In brief summary, the expression of nucleic acids encoding anti-TYRP1 or anti-B7H6 agents is typically achieved by operably linking a nucleic acid encoding the anti-TYRP1 or anti-B7H6 agent polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, γ-retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

Various promoter sequences may be used, including, but not limited to the immediate early cytomegalovirus (CMV) promoter, the CMV-actin-globin hybrid (CAG) promotor, Elongation Growth Factor-1α (EF-1α), simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, an IL-2 promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transduced or infected through viral vectors. In other aspects, the selectable marker may be carried on a separate piece of DNA and used in a sequential transduction procedure. Both selectable markers and reporter genes may be flanked with appropriate regulatory sequences to enable expression in the host cells. Useful selectable markers include, for example, antibiotic-resistance genes, such as neo and the like.

In some embodiments, the selectable marker gene comprises a nucleic acid sequence encoding truncated CD19 (trCD19).

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Transfection/Transduction

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

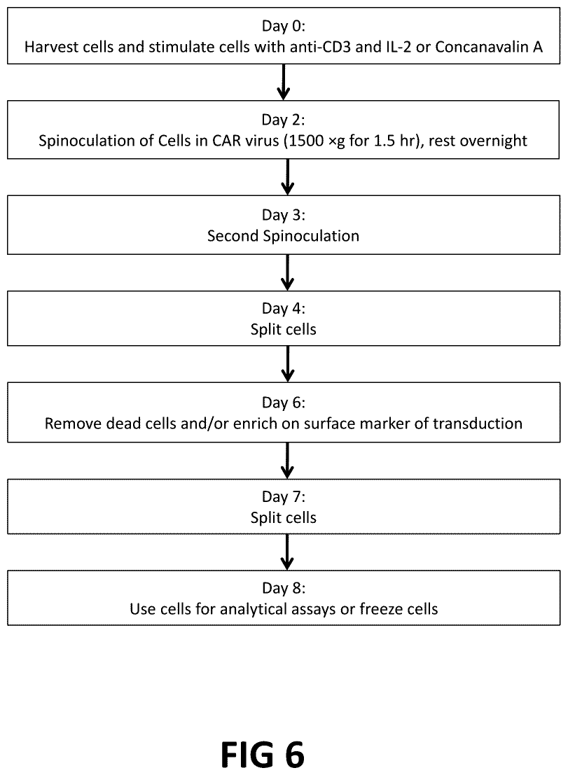

For transduction of CAR constructs to obtain CAR-expressing cells, a flow chart illustrating a potential method for manufacturing isolated CAR-expressing cells is provided in FIG. 6.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20 degrees Celsius. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., “1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Cells

Also provided are cells, cell populations, and compositions containing the cells, e.g., cells comprising a nucleic acid sequence encoding an immunostimulatory cytokine combinations and CARS of the present invention. Cells expressing immunostimulatory combination of Type 1 and/or Type 2 cytokines or cytokine fragments may be used to harvest the cytokines or cytokine fragments. The isolated cytokines or cytokine fragments may be administered to a subject or may be incorporated in a composition to be administered to a subject. Cells expressing immunostimulatory combination of Type 1 and/or Type 2 cytokines and/or the CARs of the present invention may be administered to a subject or may be incorporated in a composition to be administered to a subject. Among the compositions are pharmaceutical compositions and formulations for administration, such as for adoptive cell therapy.

Also provided are therapeutic methods for administering the immunostimulatory combination of Type 1 and/or Type 2 cytokines and/or the CARs of the present invention or the cells and compositions to subjects, e.g., patients.

### Cell Types

Thus, also provided are cells expressing the immunostimulatory combination of Type 1 and/or Type 2 cytokines and/or the CARs of the present invention.

For expressing immunostimulatory combination of Type 1 and/or Type 2 cytokines and/or the CARS of the present invention, any appropriate cells may be used. For example, cells may be: (i) prokaryotic cells, such as gram-negative bacteria and gram-positive bacteria; or (ii) eukaryotic cells, such as yeast, filamentous fungi, protozoa, insect cells, plant cells, and mammalian cells (reviewed in Frenzel A. et al. Front Immunol. 2013; 4: 217. Published online 2013 Jul. 29. doi: 10.3389/fimmu.2013.00217).

Specific examples of gram-negative bacteria that are suited for production of Ab or antigen-binding Ab fragments include, but are not limited to, E. coli, Proteus mirabilis, and Pseudomonas putidas. Specific examples of gram-positive bacteria include, but are not limited to, Bacillus brevis, Bacillus subtilis, Bacillus megaterium, Lactobacillus zeae/casei, and Lactobacillus paracasei. Specific examples of yeast bacteria that are suited for production of Ab or antigen-binding Ab fragments include, but are not limited to, Pichia pastoris, Saccharomyces cerevisiae, Hansenula polymorpha, Schizzosaccharomyces pombe, Schwanniomyces occidentalis, Kluyveromyces lactis, and Yarrowia lipolytica. Specific examples of filamentous fungi that are suited for production of Ab or antigen-binding Ab fragments include, but are not limited to, the genera Trichoderma and Aspergillus, A. niger (subgenus A. awamori), Aspergillus oryzae, and Chrysosporium lucknowense. Specific examples of protozoa that are suited for production of Ab or antigen-binding Ab fragments include, but are not limited to, Leishmania tarentolae. Specific examples of insect cells that are suited for production of Ab or antigen-binding Ab fragments include, but are not limited to, insect cell lines like Sf-9 and Sf-21 of Spodoptera frugiperda, DS2 cells of Drosophila melanogaster, High Five cells (BTI-TN-5B1-4) of Trichopulsia ni, or Schneider2 (S2) cells of D. melanogaster. They can be efficiently transfected with insect-specific viruses from the family of Baculoviridae, particularly the Autographa californica nuclear polyhedrosis virus (AcNPV). Specific examples of mammalian cells that are suited for production of Ab or antigen-binding Ab fragments include, but are not limited to, Chinese hamster ovary (CHO) cells, the human embryonic retinal cell line Per.C6 [Crucell, Leiden, Netherlands], CHO-derived cell lines such as K1-, DukXB11-, Lec13, and DG44-cell lines, mouse myeloma cells such as SP 2/0, YB 2/0, and NSO cells, GS-NSO, hybridoma cells, baby hamster kidney (BHK) cells, and the human embryonic kidney cell line HEK293, HEK293T, HEK293E, and human neuronal precursor cell line AGE1.HN (Probiogen, Berlin, Germany).

Alternatively, genetically modified organisms such as transgenic plants and transgenic animals may be used. Exemplary plants that may be used include, but are not limited to, tobacco, maize, duckweed, Chlamydomonas reinhardtii, Nicotiana tabacum, Nicotianaben thamiana, and Nicotiana benthamiana. Exemplary animals that may be used include, but are not limited to mouse, rat, and chicken.

For expressing an immunostimulatory combination of Type 1 and/or Type 2 cytokines and/or the CARs of the present invention, the cells generally are eukaryotic cells, such as mammalian cells, and typically are human cells, more typically primary human cells, e.g., allogeneic or autologous donor cells. The cells for introduction of the CAR may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

With reference to the subject to be treated with cells expressing an immunostimulatory combination of Type 1 and/or Type 2 cytokines and/or the CARs of the present invention, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

In some embodiments, the cells are T cells. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH17 cells, TH9 cells, TH22 cells, follicular helper T cells, α/β T cells, and δ/γ T cells.

In some embodiments, the cells are natural killer (NK) cells, Natural Killer T (NKT) cells, cytokine-induced killer (CIK) cells, tumor-infiltrating lymphocytes (TIL), lymphokine-activated killer (LAK) cells, or the like. In some embodiments, the cells are monocytes or granulocytes, e.g., myeloid cells, macrophages, neutrophils, dendritic cells, mast cells, eosinophils, and/or basophils. CAR-expressing phagocytic cells may be able to bind to and phagocytose or nibble target cells (Morrissey M. A. et al., Elife. 2018 Jun. 4; 7. pii: e36688. doi: 10.7554/eLife.36688).

In some embodiments, the cells are derived from cell lines, e.g., T cell lines. The cells in some embodiments are obtained from a xenogeneic source, for example, from mouse, rat, non-human primate, and pig.

Cell Acquisition for Cytokine and/or CAR Expression

For cells for expressing immunostimulatory combination of Type 1 and/or Type 2 cytokines and/or CARs of the present invention, prior to expansion and genetic modification, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and disease sites such as the fibrotic sites or tumors. In some embodiments, any number of T cell lines available and known to those skilled in the art, may be used. In some embodiments, cells can be derived from a healthy donor, from a patient diagnosed with cancer or from a patient diagnosed with an infection. In some embodiments, cells can be part of a mixed population of cells which present different phenotypic characteristics.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an Cresis or leukapheresis product. Exemplary samples include whole blood, peripheral blood mononuclear cells (PBMCs), leukocytes, bone marrow, thymus, tissue biopsy, fibrotic tissue, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

Also provided herein are cell lines obtained from a transformed cell according to any of the above-described methods. Also provided herein are modified cells resistant to an immunosuppressive treatment. In some embodiments, an isolated cell according to the invention comprises a polynucleotide encoding a CAR.

### Cell Purification

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. This would be particularly useful for isolating CAR-expressing cells. In a specific embodiment, the surface maker is trCD19. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

In some embodiments, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. For example, CD3+ T cells can be positively selected using CD3 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining TCM-enriched CD8+ T cells and CD4+ T cells further enhances efficacy. In embodiments, memory T cells are present in both CD62L+ and CD62L-subsets of CD8+ peripheral blood lymphocytes. PBMC can be enriched for or depleted of CD62L-CD8+ and/or CD62L+CD8 fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the methods include density-based cell separation methods, such as the preparation of white blood cells from peripheral blood by lysing the red blood cells and centrifugation through a Percoll or Ficoll gradient.

In any of the aforementioned separation steps, the separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

### Cell Preparation and Expansion

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation.

In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The cells of the invention can be activated and expanded, either prior to or after genetic modification of the cells, using methods as generally described, for example without limitation, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

Particularly in relation to CAR-expressing cells, T cells can be expanded in vitro or in vivo. Generally, the T cells of the invention can be expanded, for example, by contact with an agent that stimulates a CD3 TCR complex and a co-stimulatory molecule on the surface of the T cells to create an activation signal for the T cell. For example, chemicals such as calcium ionophore A23187, phorbol 12-myristate 13-acetate (PMA), or mitogenic lectins like phytohemagglutinin (PHA) can be used to create an activation signal for the T cell.

In some embodiments, T cell populations may be stimulated in vitro by contact with, for example, an anti-CD3 antibody, or antigen-binding fragment thereof, or an anti-CD2 antibody immobilized on a surface, or by contact with a protein kinase C activator (e.g., bryostatin) in conjunction with a calcium ionophore. In some embodiments, the T cell populations may be stimulated in vitro by contact with Muromonab-CD3 (OKT3). For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640® or, X-vivo 5®, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-2, IL-15, IL-21, TGF-β, and TNF, or any other additives for the growth of cells known to the skilled artisan. In a preferred embodiment, T cells are stimulated in vitro by exposure to OKT3 and IL-2. Other additives for the growth of cells include, but are not limited to, surfactant, Plasmanate, and reducing agents such as N-acetyl-cysteine and 2-mercaptoethanol. Media can include RPMI 1640®, AIM-V, DMEM, MEM, a-MEM, F-12, X-Vivo 1®, and X-Vivo 20®, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° Celsius) and atmosphere (e.g., air plus 5% CO2). T cells that have been exposed to varied stimulation times may exhibit different characteristics.

In some embodiments, the isolated cells of the invention can be expanded by co-culturing with tissue or cells. The cells can also be expanded in vivo, for example in the subject's blood after administrating the cell into the subject.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise γ-irradiated PBMC feeder cells. In some embodiments, the PBMC are irradiated with γ rays in the range of about 3000 to 3600 rads to prevent cell division. In some aspects, the feeder cells are added to culture medium prior to the addition of the populations of T cells.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° Celsius at a rate of 1 degree per minute and stored in the vapor phase of a liquid nitrogen storage tank.

Isolation of Ab or Antigen-Binding Ab Fragment from Cell Culture

Cells, such as hybridomas, that are producing Abs or antigen-binding Ab fragments of the present invention may be grown using standard methods, in suitable culture medium for this purpose (such as D-MEM or RPMI-1640), or in vivo as ascites. Abs or antigen-binding Ab fragments secreted by the cells can be separated from the culture medium, ascites fluid, or serum using conventional immunoglobulin purification procedures, such as, but not limited to, protein A-Sepharose, hydroxyapatite chromatography, gel electrophoresis, dialysis, or affinity chromatography (Ma H. et al., Methods. 2017 Mar. 1; 116:23-33. doi: 10.1016/j.ymeth.2016.11.008. Epub 2016 Nov. 18; Shukla A. A. et al. Trends Biotechnol. 2010 May; 28(5):253-61. doi: 10.1016/j.tibtech.2010.02.001. Epub 2010 Mar. 19; Arora S. et al., Methods. 2017 Mar. 1; 116:84-94. doi: 10.1016/j.ymeth.2016.12.010. Epub 2016 Dec. 22).

Methods for expressing, isolating, and evaluating multispecific and bispecific Abs and antigen-binding Ab fragments are also known in the art (for example, see Brinkmann U. et al., MAbs. 2017 February-March; 9(2): 182-212. Published online 2017 Jan. 10. doi: 10.1080/19420862.2016.1268307; Dimasi N. et al. Methods. 2018 Aug. 11. pii: S1046-2023(18)30149-X. doi: 10.1016/j.ymeth.2018.08.004).

### Therapeutic Applications

Immunostimulatory cytokine combinations, optionally in association with antibodies, receptors and other binding agents that bind target cells, e.g., tumor cells, further optionally anti-TYRP1, and/or anti-B7H6 agents (e.g., Abs, antigen-binding Ab fragments, multi-specific Abs, multi-specific antigen-binding Ab fragments, ADCs, or CARs that binds to TYRP1 or B7H6), nucleic acids encoding such an agent, vectors encoding such an agent, isolated cells obtained by the methods described above, or cell lines derived from such isolated cells, and/or pharmaceutical compositions comprising thereof can be used as a medicament in the treatment of a disease, disorder, or condition in a subject. In some embodiments, such a medicament can be used for treating a neoplastic disease or condition.

### Target Diseases and Conditions

The disease or condition may be, for example, but not limited to, cancer, preferably a solid tumor associated cancers or an infectious disorder.

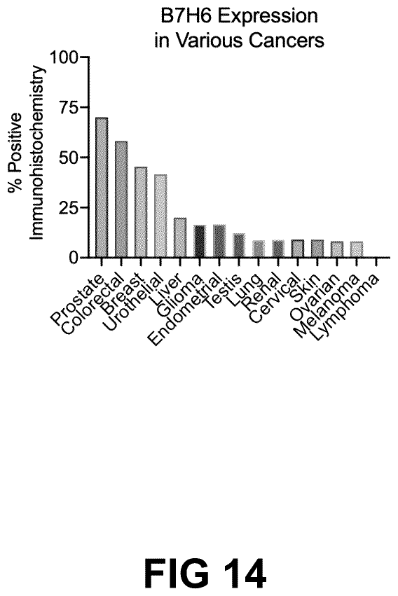

In particular embodiments, the immunostimulatory cytokine combinations, optionally in association with antibodies, receptors and other binding agents that bind target cells, e.g., tumor cells, further optionally anti-TYRP1, and/or anti-B7H6 agents may be used to treat a cancer. TYRP1 and/or B7H6 are upregulated and/or play a pathological role in a wide variety of cancers, such as, but not limited to bladder cancer, bone cancer, brain cancer, breast cancer, carcinomas, cervical cancer, colon cancer, colorectal cancer, desmoid tumor, endometrial cancer, esophageal cancer, fibromatosis, glioblastoma, head and neck cancer, liver cancer, lung cancer, lymphoma, leukemias, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, testicular cancer, thyroid cancer, urothelial cancer, and the like. The immunostimulatory cytokine combinations, optionally in association with antibodies, receptors and other binding agents that bind target cells, e.g., tumor cells, further optionally anti-TYRP1, and/or anti-B7H6 agents anti-TYRP1, or anti-B7H6 agents of the present invention may be used to treat any of the cancers above. The upregulation is particularly high in certain cancers according to FIG. 14; therefore, these cancers are preferred target diseases of the present invention. The agents of the present invention preferably may also be used to treat any other cancers in which the target cell is bound by the binding agent, e.g., TYRP1 and/or B7H6 are upregulated or have a pathological role.

In certain embodiments, the immunostimulatory cytokine combinations, optionally in association with antibodies, receptors and other binding agents that bind target cells, e.g., tumor cells, further optionally anti-TYRP1, and/or anti-B7H6 agents may be used to treat a non-cancer disease or condition such as, but not limited to, infectious disease, (e.g., virus, bacteria, yeast, fungus, or parasite). B7H6 is upregulated and/or plays a pathological role in many other diseases and conditions, particularly viral infections (Journal of Molecular Medicine volume 98, pages 135-148(2020)). Additionally, IL-2 is part of the body's natural response to microbial infection. Furthermore, T cells generally orchestrate immune response to kill pathogen-infected cells. Therefore, the agents of the present invention may be utilized to infected diseases.

### Subject

The subject referred to herein may be any living subject. In a preferred embodiment, the subject is a mammal. The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

In some embodiments, the subject, to whom the immunostimulatory cytokine combinations, Abs, antigen-binding Ab fragments, CAR expressing cells, cells, cell populations, or compositions are administered is a primate, such as a human. In some embodiments, the primate is a monkey or an ape. The subject can be male or female and can be any suitable age, including infant, juvenile, adolescent, adult, and geriatric subjects. In some examples, the patient or subject is a validated animal model for disease, adoptive cell therapy, and/or for assessing toxic outcomes such as cytokine release syndrome (CRS).

In some embodiments, the subject has persistent or relapsed disease, e.g., following treatment with another immunotherapy and/or other therapy. In some embodiments, the administration effectively treats the subject despite the subject having become resistant to another therapy. In some embodiments, the subject has not relapsed but is determined to be at risk for relapse, such as at a high risk of relapse, and thus the compound or composition is administered prophylactically, e.g., to reduce the likelihood of or prevent relapse.

### Cell Origin

For purposes of the methods of the therapy wherein the immunostimulatory cytokine combination is expressed in a recombinant cell, and host cells or populations of cells are administered, the cells can be cells that are xenogeneic, allogeneic or autologous to the subject. Generally, the cells are autologous to the subject.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

Cells expressing immunostimulatory cytokine combinations, optionally in association with antibodies, receptors and other binding agents that bind target cells, e.g., tumor cells, further optionally anti-TYRP1, and/or anti-B7H6 agents, e.g., antigen-binding Ab fragments such as scFvs, or a composition comprising such may also be administered to a subject. In some embodiments, B cells or plasma cells expressing immunostimulatory cytokine combinations, optionally in association with antibodies, receptors and other binding agents that bind target cells, e.g., tumor cells, further optionally anti-TYRP1, and/or anti-B7H6 agents, antigen-binding Ab fragments, or a composition comprising such may be adoptively transferred.

### Functional Activity

In one embodiment, the present invention includes a type of cellular therapy where isolated cells are genetically modified to express a combination of Type 1 and/or Type 2 cytokines and/or a CAR against TYRP1 or B7H6, and the CAR cell is infused into a subject in need thereof. Such administration can promote activation of the cells (e.g., T cell activation) in a target molecule specific manner, such that the cells of the disease or disorder are targeted for destruction. In the case where the cell is a T cell, cells, such as CAR T cells, are able to replicate in vivo resulting in long-term persistence that may lead to sustained control of diseases, disorders, or conditions associated with TYRP1 or B7H6 or other target antigen expressed or overexpressed on target cells, e.g., cancer, preferably one associated with solid tumors or infection.

In one embodiment, the isolated cells of the invention can undergo in vivo expansion and can persist for an extended amount of time. In another embodiment, where the isolated cell is a T cell, the isolated T cells of the invention evolve into specific memory T cells that can be reactivated to inhibit growth of any additional target molecule expressing cells. T cells may differentiate in vivo into a central memory-like state upon encounter and subsequent elimination of target cells expressing the surrogate antigen. Similarly, in certain embodiments, where the isolated cells is a B cell, the isolated B cells may evolve into memory B cells that can be reactivated to inhibit growth of any additional target molecule expressing cells.

Without wishing to be bound by any particular theory, the immune response elicited by the isolated immunostimulatory combinations of Type 1 and/or Type 2 cytokines, and/or anti-TYRP1, and/or anti-B7H6 agent-modified immune cells may be an active or a passive immune response. In addition, the immune response may be part of an adoptive immunotherapy approach in which immunostimulatory cytokine combinations, and/or anti-TYRP1, and/or anti-B7H6 agent-modified immune cells induce an immune response specific to the AB domain of the anti-TYRP1 or anti-B7H6 agent.

In certain embodiments, immunostimulatory combination of Type 1 and/or Type 2 cytokines, and/or anti-TYRP1, and/or anti-B7H6 agent-expressing cells are modified in any number of ways, such that their therapeutic or prophylactic efficacy is increased. For example, the immunostimulatory combination of Type 1 and/or Type 2 cytokines, and/or anti-TYRP1, and/or anti-B7H6 agent may be conjugated either directly or indirectly through a linker to a targeting moiety. The practice of conjugating compounds, e.g., the CAR, to targeting moieties is known in the art. See, for instance, Wadwa et al., J. Drug Targeting 3: 1 1 1 (1995), and U.S. Pat. No. 5,087,616.

Once the cells are administered to a subject (e.g., a human), the biological activity of the engineered cell populations and/or antibodies in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain mediators, such as GM-CSF, IL-6, RANTES (CCL5), TNF-α, IL-4, IL-10, IL-13, IFN-γ, granzyme B, perforin, CD107a, or IL-2.

In some aspects the biological activity is measured by assessing clinical outcome, such as the reduction in disease symptoms. In case of autoimmune diseases, decrease in autoreactive T cells, B cells, or Abs and reduced inflammation may represent successful biological activity. In case of cancer, improved efficacy may be shown by better infiltration of disease-resolving immune cells into the tumor, reduced tumor sizes, or reduced ascites. In some embodiments, gene expression profiles may be also investigated to evaluate the activity.

### Target Cells

Cells that may be targeted by any immunostimulatory combination of Type 1 and/or Type 2 cytokines, and/or anti-TYRP1, and/or anti-B7H6 agents of present invention include any TYRP1- or B7H6-expressing cells or cells associated with diseases or conditions described herein. The target cell may be present in any part of the body of a subject, including blood or lymphatic circulation, and disease-affected tissues, preferably a solid tumor. For example, when the target disease comprises a solid tumor, the disease-affected tissues include, but are not limited to, bladder, bone, brain, breast, cervical, colon, connective tissue, dermis, desmoid, endometrial tissue, epidermis, esophagus, glial, liver, lung, neuron, oral, oesophago-gastric, stomach, oligodendritic, oral tissue, oral squamous cells, ovary, pancreas, prostate, rectum, renal, skin, squamous cells, stomach, subcutaneous connective tissue, testicular, thyroid, and urothelial. Alternatively, target cells may blood cells or hematopoietic cells.

Preferably, the immunostimulatory combination of Type 1 and/or Type 2 cytokines, and/or optionally in association with antibodies, receptors and other binding agents that bind target cells, e.g., tumor cells, further optionally anti-TYRP1, and/or anti-B7H6 agents of the invention are used to treat cancer, wherein TYRP1 or B7H6 or other antigen is upregulated or overexpressed. In particular, the cells of the invention may be used to treat bladder cancer, bone cancer, brain cancer, breast cancer, carcinomas, cervical cancer, colon cancer, colorectal cancer, desmoid tumor, endometrial cancer, esophageal cancer, fibromatosis, glioblastoma, head and neck cancer, liver cancer, lung cancer, lymphoma, leukemias, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, testicular cancer, thyroid cancer, urothelial cancer, and the like.

### Pharmaceutical Compositions

The compositions of the present invention may be administered in a number of ways depending upon whether local or systemic treatment is desired.

In general, administration may be topical, parenteral, or enteral.

As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. In a preferred embodiment, parenteral administration of the compositions of the present invention comprises subcutaneous or intraperitoneal administration.

The terms “oral”, “enteral”, “enterally”, “orally”, “non-parenteral”, “non-parenterally”, and the like, refer to administration of a compound or composition to an individual by a route or mode along the alimentary canal. Examples of “oral” routes of administration of a composition include, without limitation, swallowing liquid or solid forms of a composition from the mouth, administration of a composition through a nasojejunal or gastrostomy tube, intraduodenal administration of a composition, and rectal administration, e.g., using suppositories for the lower intestinal tract of the alimentary canal.

Compositions of the present invention may be suited for topical, parenteral, or enteral administration.

Preferably, formulated compositions comprising soluble proteins, Abs, antigen-binding Ab fragments, ADCs, or CARs, polynucleotides or vectors encoding such, or cells expressing thereof are suitable for administration via parenteral administration for example, subcutaneous, intramuscular, intraperitoneal or intravenous injection.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release. Such formulation may be, for example, made of a biodegradable, biocompatible polymer, such as, but not limited to, ethylene vinyl acetate, poly(alkyl cyanoacrylates), poly(anhydrides), poly(amindes), poly(ester), poly(ester amindes), poly(phosphoesters), polyglycolic acid (PGA), collagen, polyorthoester, polylactic acid (PLA), poly(lactic-co-glycolidc acid) (PLAGA), or naturally occurring biodegradable polymers such as chitosan and hyaluronic acid-based polymers (Kamaly N. et al, Chem Rev. Author manuscript; available in PMC 2017 Jul. 13).

Pharmaceutical compositions and formulations for topical administration may include transdermal patches, ointments, lotions, creams, gels, drops, suppositories, sprays, liquids, semi-solids, monophasic compositions, multiphasic compositions (e.g., oil-in-water, water-in-oil), foams, microsponges, liposomes, nanoemulsions, aerosol foams, polymers, fullerenes, and powders. Conventional pharmaceutical carriers, aqueous, powder or oily bases, thickeners and the like may be necessary or desirable.

Compositions and formulations for parenteral, intrathecal, or intraventricular administration may include sterile aqueous solutions that may also contain buffers, diluents and other suitable additives such as, but not limited to, penetration enhancers, carder compounds and other pharmaceutically acceptable carriers or excipients.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical compositions of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated to provide appropriate in vivo distribution of the active ingredient. In many cases, concentrating the distribution of an anti-tumor drug in the tumor site is challenging, and it can be so even when a drug has a specificity to a molecule expressed by cancer cells. Various strategies have been developed to address the issue and any appropriate strategies may be applied for the current invention (for example, reviewed in Rosenblum D. et al., Nat Commun. 2018 Apr. 12; 9(1):1410. doi: 10.1038/s41467-018-03705-y). For delivering a drug to the brain, the drug needs to cross the blood-brain barrier (BBB). Any appropriate strategies to enable BBB crossing may be utilized to for the delivery of any of the immunostimulatory cytokine combinations (see for example, Dong X. et al., Theranostics. 2018; 8(6): 1481-1493, for exemplary strategies).

The compositions of the present invention may be formulated into any of many possible dosage forms such as, but not limited to, tablets, capsules, liquid syrups, soft gels, suppositories, aerosols, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Formulations comprising any of the immunostimulatory combination of Type 1 and/or Type 2 cytokines, of the present invention or populations of cells expressing any of the immunostimulatory cytokine combinations, and/or optionally in association with antibodies, receptors and other binding agents that bind target cells, e.g., tumor cells, further optionally anti-TYRP1, and/or anti-B7H6 agents such as anti-TYRP1 or anti-B7H6 CARs of the present invention may include pharmaceutically acceptable excipient(s). Excipients included in the formulations will have different purposes depending, for example, on the CAR construct, the subpopulation of cells used, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents. The formulations comprising populations of the CAR-expressing cells of the present invention will typically have been prepared and cultured in the absence of any non-human components, such as animal serum (e.g., bovine serum albumin).

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs. Such agents or drugs may be, but are not limited to, an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an anti-fibrotic drug, an immunosuppressive drug, a steroid, a bronchodilator, a beta blocker, a matrix metalloproteinase inhibitor, Type 1 and/or Type 2 cytokines or cytokine fragment of the present invention, an enzyme, a hormone, a neurotransmitter, a toxin, a compound, a small molecule, a small molecule inhibitor, a protein, a peptide, a vector, a plasmid, a viral particle, a nanoparticle, a DNA molecule, an RNA molecule, an siRNA, an shRNA, a micro RNA, an oligonucleotide, or an imaging drug. Specific examples are, for instance, but are not limited to, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

### Kits

Also provided herein are kits comprising (a) one or more of immunostimulatory combination of Type 1 and/or Type 2 cytokines or immunostimulatory cytokine combinations, and/or anti-TYRP1, and/or anti-B7H6 agents (Abs, antigen-binding Ab fragments, ADCs, CARs), polynucleotides encoding such, vectors encoding such, cells expressing such; and (b) for example an instruction for use in treating or diagnosing a disease or condition described herein. The kit may include a label indicating the intended use of the contents of the kit. The term “label” as used herein includes any written materials, marketing materials, or recorded materials supplied on, with, in, or appended to the kit.

### Method of Administration

The administration route used in the method of the present invention may be any appropriate route, which depends upon whether local or systemic treatment is desired.

In general, administration may be topical, parenteral, or enteral.

Preferably, formulated compositions comprising soluble protein, Abs, antigen-binding Ab fragments, ADCs, or CARs, polynucleotides or vectors encoding such, cells expressing such may be administered parenterally, for example, via subcutaneous, intramuscular, intraperitoneal or intravenous injection.

In the case of adoptive cell therapy, methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the composition of the present invention may be administered using any appropriate medical devices (for example, reviewed in Richter B. B., J. BioDrugs (2018) 32: 425).

### Dosing

For administration of any of the immunostimulatory combination of Type 1 and/or Type 2 cytokines and/or the CARS and compositions of the present invention, the dosage will vary and depend on, for example, the target disease, the severity of the disease, the route of administration, and pharmacokinetic factors. Dosing may be modified based on the response observed in the subject.

For administration of any of the immunostimulatory combination of Type 1 and/or Type 2 cytokines, and/or anti-TYRP1, and/or anti-B7H6 Abs, antigen-binding Ab fragments, or ADCs, or compositions comprising such, appropriate dosage regimen may be determined using any appropriate methodology (for example, Bai S. et al., Clin Pharmacokinet. 2012 Feb. 1; 51(2):119-35. doi: 10.2165/11596370-000000000-00000).

In some embodiments, the dosage may be from about 1 ng/kg to about 1 g/kg (of the body weight of a subject) per day. In some aspects, the dose may be from about 10 ng/kg/day to about 900 mg/kg/day, from about 20 ng/kg/day to about 800 mg/kg/day, from about 30 ng/kg/day to about 800 mg/kg/day, from about 40 ng/kg/day to about 700 mg/kg/day, from about 50 ng/kg/day to about 600 mg/kg/day, from about 60 ng/kg/day to about 500 mg/kg/day, from about 70 ng/kg/day to about 400 mg/kg/day, from about 80 ng/kg/day to about 300 mg/kg/day, from about 90 ng/kg/day to about 200 mg/kg/day, or from about 100 ng/kg/day to about 100 mg/kg/day. The treatment may be repeated or periodically given to a subject for days, months, or years, or until the desired effect is achieved. An exemplary dosing regimen include administering an initial dose of an immunostimulatory combination of Type 1 and/or Type 2 cytokines, and/or optionally in association with antibodies, receptors and other binding agents that bind target cells, e.g., tumor cells, further optionally anti-TYRP1, and/or anti-B7H6 agents; i.e., Abs, antigen-binding Ab fragments, or ADCs of about 2 mg/kg, followed by a weekly maintenance dose of about 1 mg/kg.

Dosing frequency may be, for example, three times per day, twice per day, once per day, every other day, once per week, every other week, once per three weeks, once per four weeks, once per five weeks, once per six weeks, once per seven weeks, once per eight weeks, once per nine weeks, once per ten weeks, once per three months, once per four months, once per six months, once per year, or even less frequent.

The pharmaceutical composition in some embodiments contains cells expressing the immunostimulatory combination of Type 1 and/or Type 2 cytokines and/or the CAR of the present invention in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of genetically engineered cells expressing an immunostimulatory combination of Type 1 and/or Type 2 cytokines and/or the CAR of the present invention, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. For example, in some embodiments the administration of the cells or population of cells can comprise administration of about 10³ to about 10¹ cells per kg body weight including all integer values of cell numbers within those ranges.

The cells or population of cells can be administrated in one or more doses. In some embodiments, said effective amount of cells can be administrated as a single dose. In some embodiments, said effective amount of cells can be administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection into the disease site.

For purposes of the invention, the amount or dose of the inventive immunostimulatory combination of Type 1 and/or Type 2 cytokines administered should be sufficient to effect a therapeutic or prophylactic response in the subject or animal over a reasonable time frame. For example, the dose of the inventive cytokine combination should be sufficient to bind to target cell, or antigen, or detect, treat or prevent disease in a period of from about 2 hours or longer, e.g., about 12 to about 24 or more hours, from the time of administration. In certain embodiments, the time period could be even longer. The dose will be determined by the efficacy of the particular inventive immunostimulatory combination of Type 1 and/or Type 2 cytokines, the route of administration, and the condition of the animal (e.g., human), as well as the body weight of the animal (e.g., human) to be treated.

For purposes of the invention, an assay, which comprises, for example, comparing the extent to which target cells are lysed or, in the context of CARs, IFN-γ is secreted by T cells expressing the inventive CAR, polypeptide, or protein upon administration of a given dose of such T cells to a mammal, among a set of mammals of which is each given a different dose of the T cells, could be used to determine a starting dose to be administered to a mammal. The extent to which target cells are lysed or IFN-γ is secreted upon administration of a certain dose can be assayed by methods known in the art.

In some embodiments, the immunostimulatory cytokine combination or compositions of the present invention are administered as part of a combination treatment, such as simultaneously with or sequentially with, in any order, another therapeutic intervention, such as an antibody or engineered cell or receptor or agent, such as a cytotoxic or therapeutic agent. The cells or antibodies in some embodiments are co-administered with one or more additional therapeutic agents or in connection with another therapeutic intervention, either simultaneously or sequentially in any order. In some contexts, the immunostimulatory cytokine combination or compositions are co-administered with another therapy sufficiently close in time such that the immunostimulatory cytokine combination or compositions enhance the effect of one or more additional therapeutic agents, or vice versa. In some embodiments, the cells or antibodies are administered prior to the one or more additional therapeutic agents. In some embodiments, the immunostimulatory cytokine combination is administered after the one or more additional therapeutic agents. Furthermore, the compositions of the present invention may be given to a subject along with one or more of other therapies, which may be surgery, or a radiotherapy.

In some embodiments, in CAR T therapy, a lymphodepleting chemotherapy is administered to the subject prior to, concurrently with, or after administration (e.g., infusion) of CAR cells. In an example, the lymphodepleting chemotherapy is administered to the subject prior to administration of the cells. For example, the lymphodepleting chemotherapy ends 1-4 days (e.g., 1, 2, 3, or 4 days) prior to CAR cell infusion. In embodiments, multiple doses of CAR cells are administered, e.g., as described herein. In embodiments, a lymphodepleting chemotherapy is administered to the subject prior to, concurrently with, or after administration (e.g., infusion) of a CAR-expressing cell described herein. Examples of lymphodepletion include, but may not be limited to, nonmyeloablative lymphodepleting chemotherapy, myeloablative lymphodepleting chemotherapy, total body irradiation, etc. Examples of lymphodepleting agents include, but are not limited to, antithymocyte globulin, anti-CD3 antibodies, anti-CD4 antibodies, anti-CD8 antibodies, anti-CD52 antibodies, anti-CD2 antibodies, TCRαβ blockers, anti-CD20 antibodies, anti-CD19 antibodies, Bortezomib, rituximab, anti-CD154 antibodies, rapamycin, CD3 immunotoxin, fludarabine, cyclophosphamide, busulfan, melphalan, Mabthera, Tacrolimus, alefacept, alemtuzumab, OKT3, OKT4, OKT8, OKT11, fingolimod, anti-CD40 antibodies, anti-BR3 antibodies, Campath-1H, anti-CD25 antibodies, calcineurin inhibitors, mycophenolate, and steroids, which may be used alone or in combination.

### EXAMPLES

### Example 1: Generation and Expression of CARs, Cytokines, and CAR T Cells

Construction of CAR Vectors:

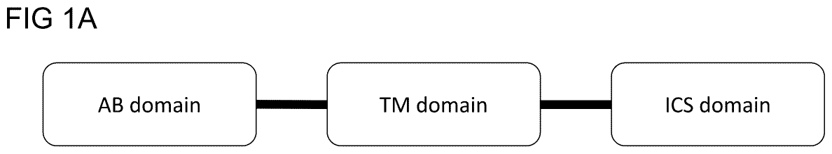

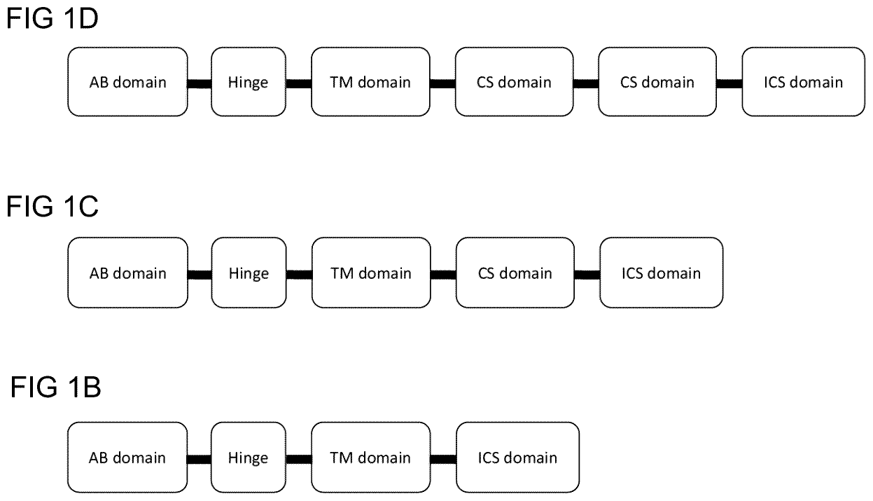

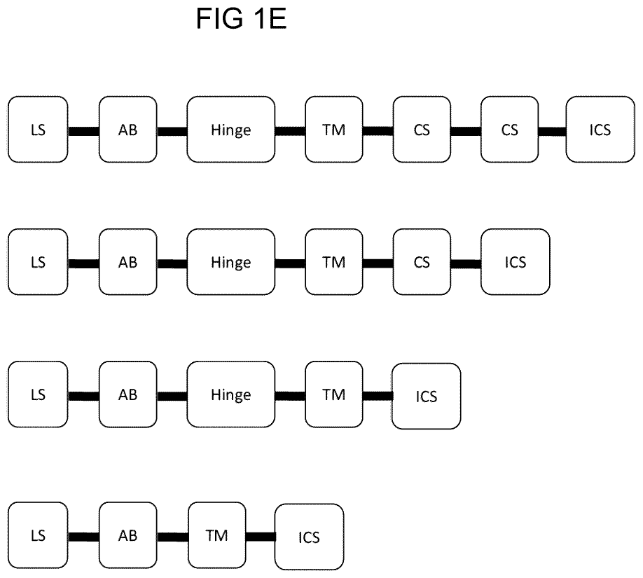

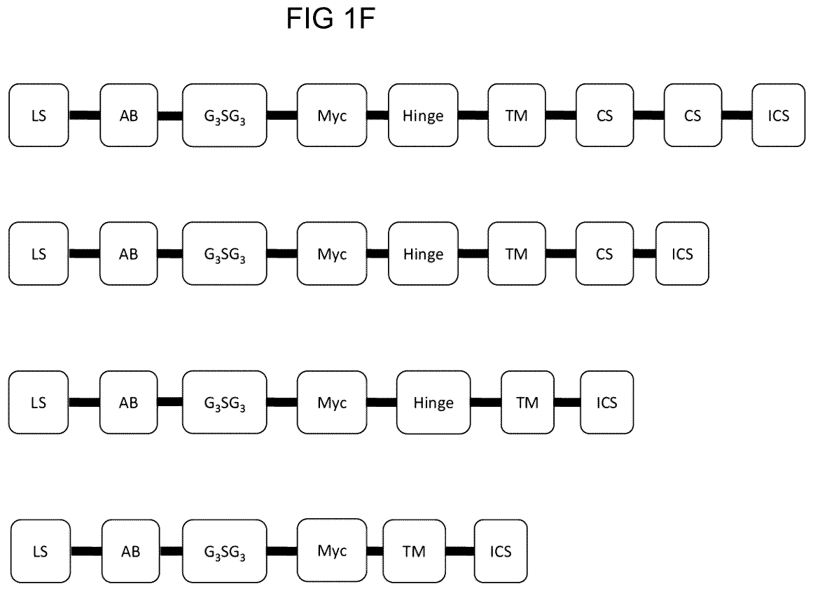

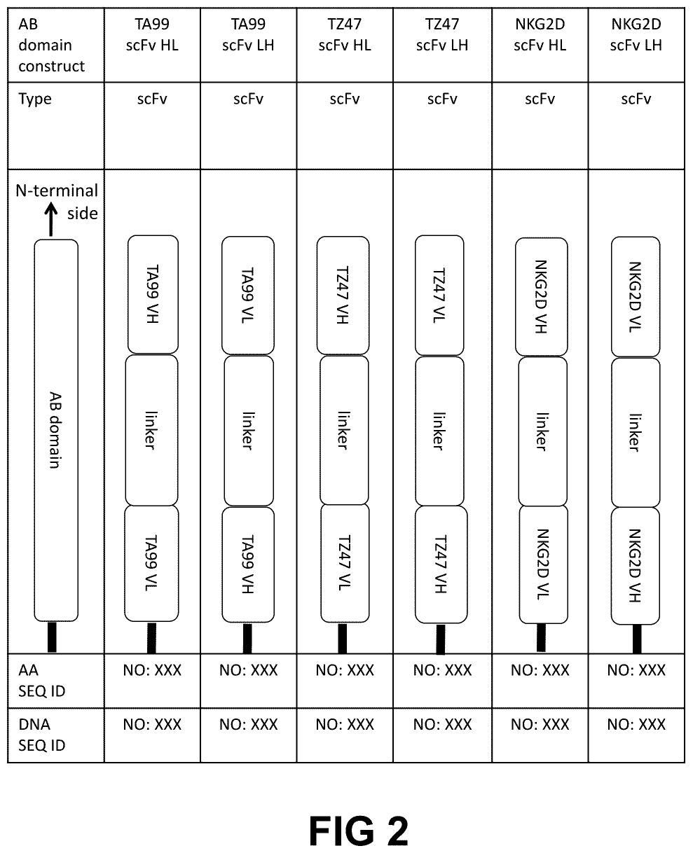

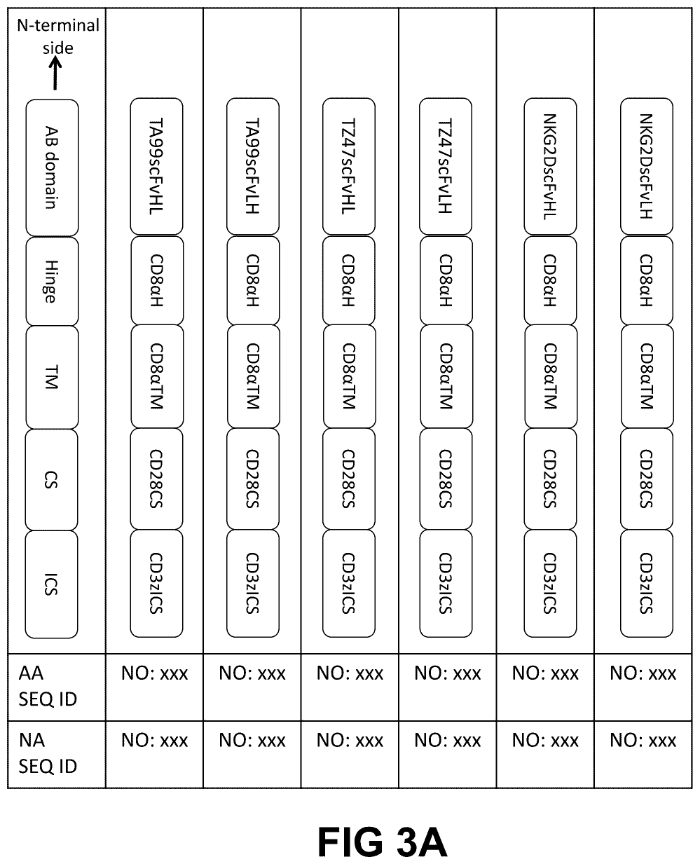

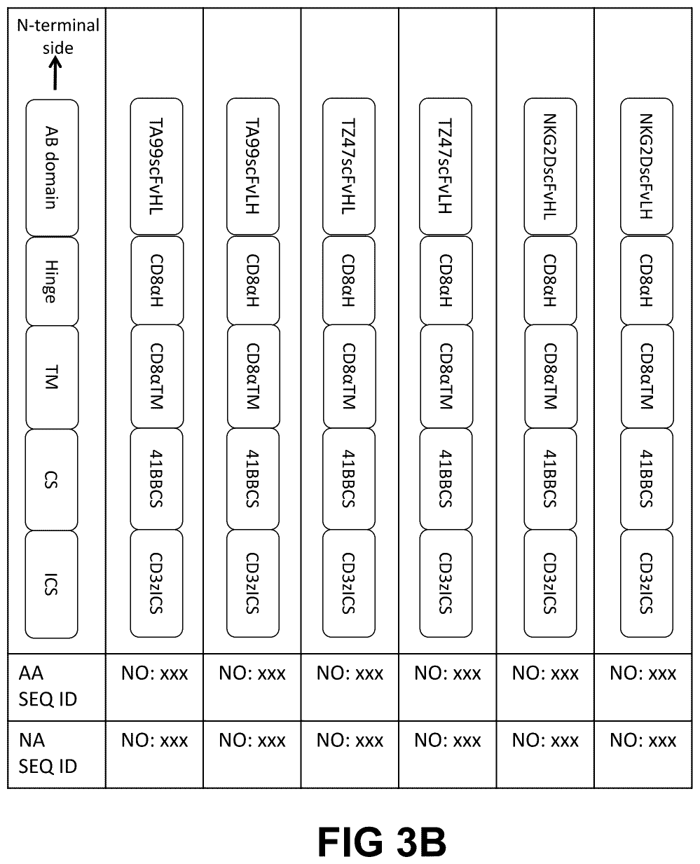

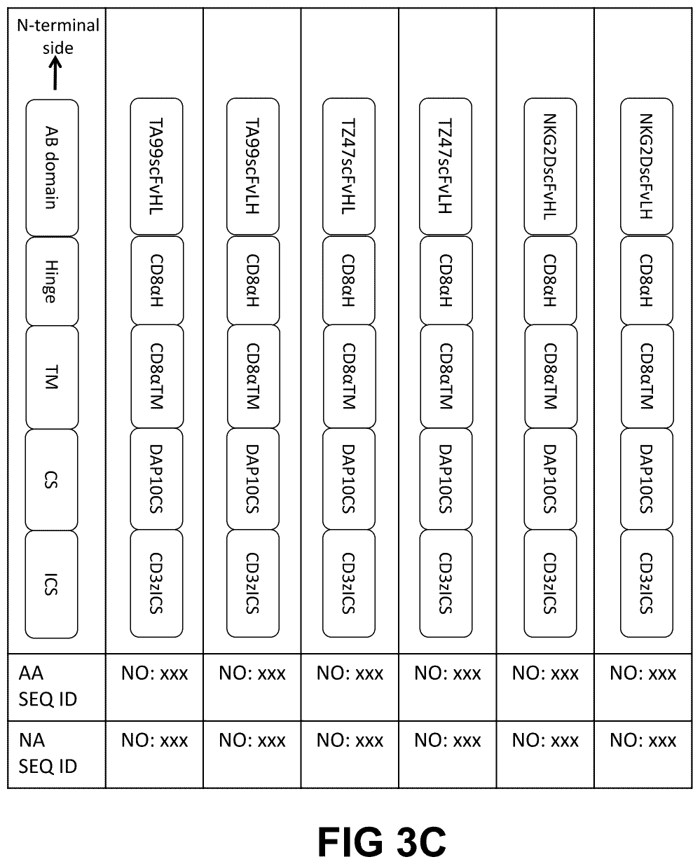

Exemplary TA99, TZ47, and NKG2D CAR constructs were created according to the schematics depicted in FIG. 1, FIG. 2, and FIG. 3.

TA99 ScFv CAR coding sequence was designed and then ordered from Genewiz.

The TA99 ScFv CAR coding sequence was subcloned in frame with mouse CD8 transmembrane and mouse CD28 and CD3ζ cytoplasmic domains into the pCMV2.1 retroviral vector, which contains an internal ribosomal entry sequence (IRES) to allow green fluorescence protein (GFP) co-expression. The TZ.47 ScFv CAR coding sequence was cloned in frame with the human CD28 transmembrane and cytoplasmic domain and the CD3ζ cytoplasmic domain into the pSFG vector with a truncated mouse CD19 marker. The mouse type 2 transmembrane protein NKG2D was cloned in frame with mouse CD3ζ cytoplasmic domain into the pFB-Neo vector.

Construction of Cytokine Vectors:

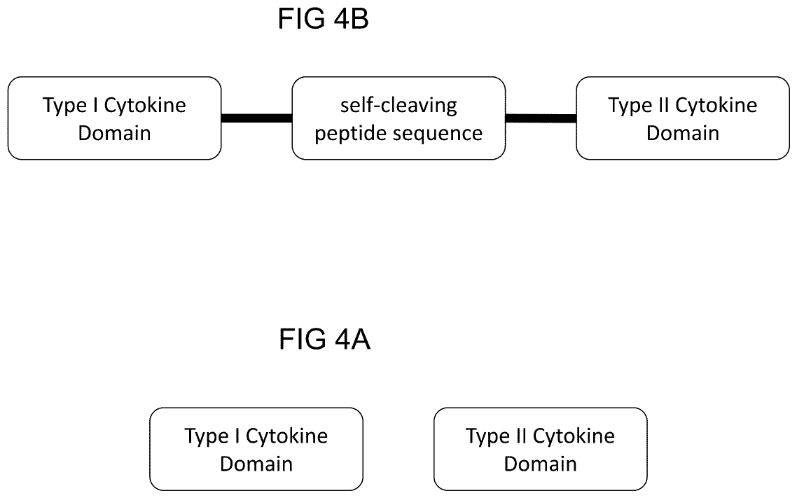

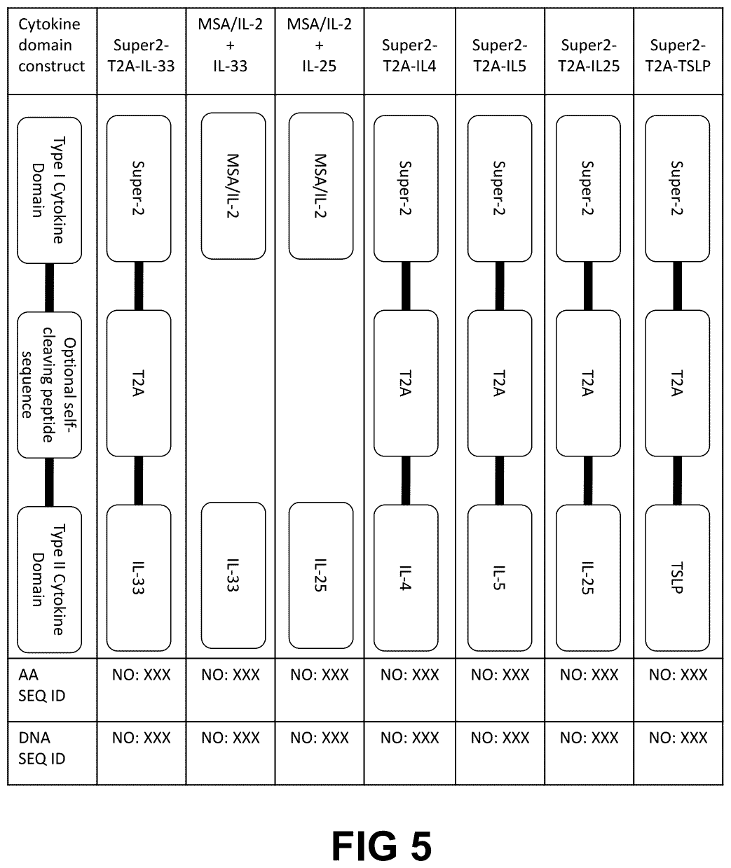

Exemplary cytokine constructs were created according to the schematics depicted in FIG. 4 and FIG. 5.

Super2 and IL33, IL4, IL5, IL25, or TSLP cytokine coding sequences were cloned separately or in tandem with an intervening T2A self-cleaving peptide sequence into a pCMV2.1 vector.

Retroviral Packaging:

Retroviruses were packaged by first plating 2.5×10⁶ HEK 293T cells on a 10 cm plate in 10 mL of Complete DMEM media 18-hours prior to transfection. To produce ecotropic virus, cells were transfected using the calcium phosphate transfection method with 10 μg of CAR or cytokine plasmid and 10 μg of pCL-Eco packaging plasmid. Media was replaced with fresh media approximately 8 hours post calcium phosphate treatment. Viral supernatants were harvested 48 hours post transfection and filtered through a 0.45 μM filter 48 hours post transfection and stored at −80° C. until use.

Generation of mouse CAR T cells:

Primary mouse T cells were cultured in Complete RPMI 1640 media (RPMI-1640 supplemented with 10% heat-inactive fetal bovine serum, 10 mM HEPES, 0.1 mM non-essential amino acids, 1 mM sodium pyruvate, 100 U/mL penicillin, 100 μg/mL streptomycin and 50 μM of 2-mercaptoethanol).

Splenocytes from C57BL/6 mice, congenically marked CD45.1 B6 mice, IFNγ KO, or Prf KO mice were transduced 18-24 hrs after Concanavalin A (1 μg/ml; Sigma) stimulation and cultured in Complete RPMI media plus 25 U/ml of IL-2. Mouse T cells were transduced with retrovirally encoded cytokine supernatant by resuspending activated T cells in viral supernatant and polybrene (1 ug/ml; Sigma) at 8 million cells per well in 24 well plates followed by spinoculation at 1500×g for 90 mins at 37° C. Approximately 24 hours later, cells were transduced a second time to express the CAR receptor as performed on the previous day. Two days post-infection, cells were analyzed by flow cytometry to determine CAR transduction efficiency by Protein L staining and cytokine transduction efficiency by GFP expression before administration to mice.

To measure cytokine production, 1×10\hat{ }5 CAR T cells were co-cultured with tumor cells for 24 h at 37° C. Tumor cell lines were plated at a 1:1 and 0.5:1 Effector:Target ratio. Cell-free medium was collected and analyzed for IL-4, IL-5, IL-25, IL-33 and TSLP using ELISAs or LEGENDplex™ assays (Biolegend) as described in the manufacturer's protocol.

CAR T cells were characterized prior to adoptive transfer into tumor challenged mice by flow cytometry following staining with antibodies specific for CD3E (145-2c11, Biolegend), and CD4 (GK1.5, Biolegend) and biotinylated Protein L (Catalog No. 29997 Pierce) followed by streptavidin-PE (Life Technologies/Invitrogen) to detect scFv CAR expression. Samples were analyzed on an Accuri C6 Cytometer (BD Biosciences).

The aforementioned materials were utilized for experiments described in the remaining examples. When applicable, the data generated from these experiments was analyzed using the following statistical methods. For all in vitro assays, experiments were run as biological triplicates. Error bars for experiments represent standard deviations, except error bars for measurements of tumor area, which represent SEM. All repeat experiments were combined for statistical analysis unless otherwise stated. For in vivo mouse efficacy studies, each independent experiment included five mice per experimental group unless otherwise specified. Data from two independent in vivo mouse experiments were combined for analyses. Survival was graphed on Kaplan-Meier plots and the Log-rank Mantel-Cox test was used to assess statistical significance. ANOVA with Dunnett's Test was used to assess statistical significance of in vivo tumor measurements and compared to control. Two-way analysis of variance (ANOVA) was used to analyze differences between CAR T cell experimental groups with appropriate post-hoc analysis. Assessment of mean fluorescence intensity (MFI) differences in CAR T cells was analyzed using Tukey's Test. All statistical analyses were run for two-sided comparisons under the assumption of a normal distribution. Statistical analysis was assessed through GraphPad Prism software.

### Example 2: In Vivo Efficacy of CAR T Cells with Cytokine Constructs in a B16F10 Melanoma Model

Melanoma tumor challenge in C57BL/6J Mice:

Mice that were 7-8 weeks old were purchased from Charles River Laboratories and then injected intradermally with 0.2×10⁶ B16F10 tumors on day 0. Six days post tumor inoculation, mice were treated by intravenous injection of 7×10⁶ CAR T cells expressing (i) Super-2-T2A-IL-33 cytokine construct and TA99 CAR, (ii) TA99 CAR only, (iii) Super-2 and TA99 CAR, (iv) IL-33 and TA99 CAR, or (v) no treatment. Tumor size was monitored every two days until end point or when tumors reached 15 mm in diameter. Results are provided in FIG. 7.

Results indicate that tumor growth inhibition of B16F10 melanoma in FIG. 7A and extension of survival in FIG. 7B were observed only after treatment with cells expressing TA99 CAR and the Super-2-T2A-IL-33 cytokine construct. The remainder of conditions did not appreciably affect tumor growth. Most notably, treatment with TA99 CAR T cells expressing Super-2 OR IL-33 resulted in little to no effect on B16F10 growth kinetics compared to no treatment or TA99 CAR T cells without exogenous cytokines. When Super-2 and IL-33 are co-expressed in tandem, separated by a T2A sequence (self-cleaving peptide), TA99 CAR T cells provide synergistic enhancement of tumor control compared to either cytokine alone.

### Example 3: In Vivo Efficacy of CAR T Cells with Cytokine Constructs in a B16F10 Metastatic Lung Tumor Model

Metastatic lung tumor challenge in C57BL/6J mice:

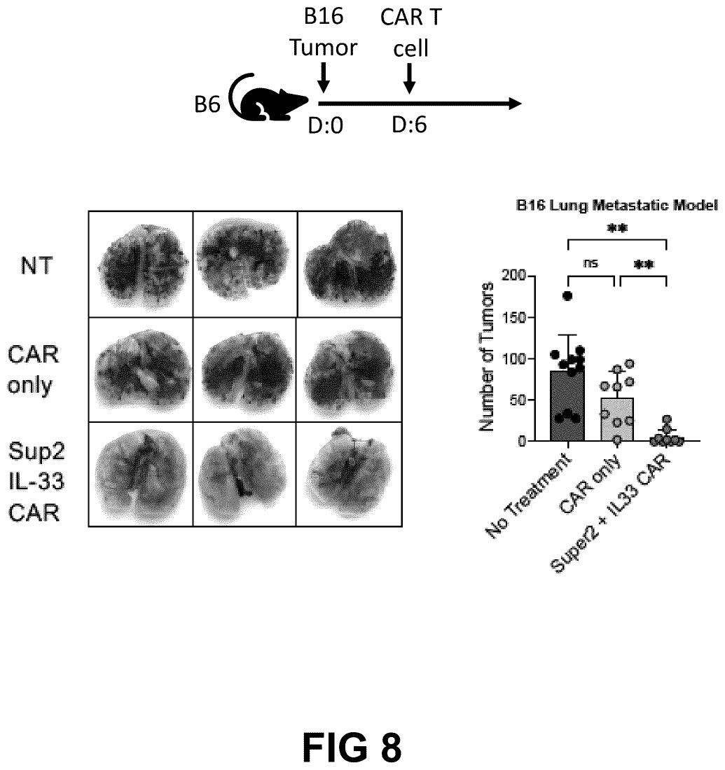

For the metastatic tumor model, 2×10\hat{ }5 B16F10 cells in 200 μL of HBSS were injected i.v. through the tail vein of C57Bl/6 mice ˜8 weeks. After the tumors established for 6 days, 7×10\hat{ }6 mouse CAR T cells expressing (i) Super-2-T2A-IL-33 cytokine construct and TA99 CAR (Super2+IL33 CAR T), (ii) TA99 CAR only, or (iii) no treatment were injected through the tail vein. Lungs of the mice baring lung metastatic tumors were removed and day 15 and lung tumors were analyzed. Results are provided in FIG. 8.

Results indicate only a minor reduction in the number of lung tumors after treatment with TA99 CAR. Significantly fewer tumors were observed after treatment with Super-2-T2A-IL-33 cytokine construct and TA99 CAR T cells, indicating enhancement of tumor control compared to CAR alone and demonstrate the beneficial effects of the subject immunostimulatory cytokine combination on antitumor immunity, particularly antitumor immune responses against solid tumors.

### Example 4: In Vivo Efficacy of Recombinant Cytokine Combinations in a B16F10 Melanoma Model in C57BL/6J Mice

Melanoma Tumor Challenge in C57BL/6J Mice:

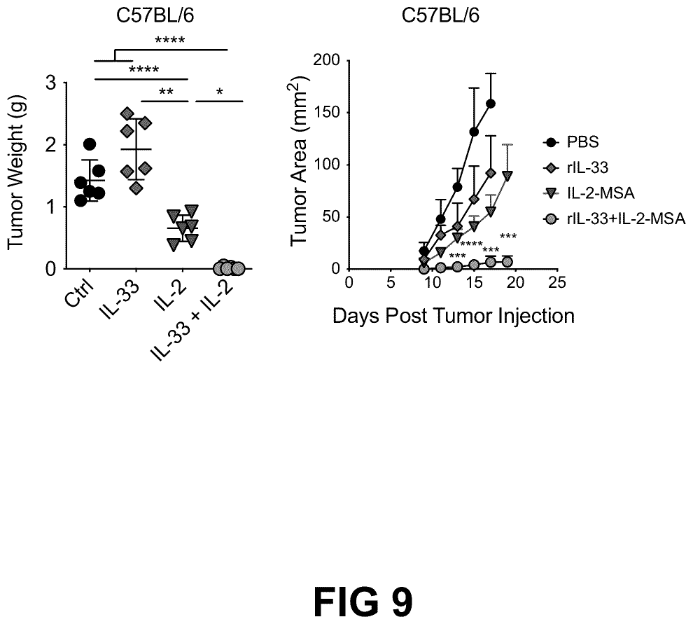

Melanoma tumors were generated in C57BL/6J mice described in Example 2. Six days post tumor inoculation, mice were treated by intraperitoneal injection of (i) a combination of recombinant cytokines IL-33 and MSA/IL-2, (ii) recombinant IL-33, (iii) recombinant MSA/IL-2, or (iv) PBS control. Tumor size was monitored every two days until end point or when tumors reached 15 mm in diameter. Results are provided in FIG. 9.

Results of FIG. 9 indicate that intraperitoneal injection of recombinant cytokines (IL-2-MSA and rIL-33) promotes anti-tumor response in the B16F10 melanoma model in C57BL/6 mice.

### Example 5: In Vivo Efficacy of Recombinant Cytokine Combinations in a B16F10 Melanoma Model in Rag2-Deficient (Rag2−/−) Mice

Melanoma Tumor Challenge in Rag2-Deficient Mice:

Melanoma tumors were generated in Rag2-deficient (Rag2−/−) mice, which lack T cells, as described in Example 2. Six days post tumor inoculation, mice were treated by intraperitoneal injection of (i) a combination of recombinant cytokines IL-33 and IL-2, (ii) recombinant IL-33, (iii) recombinant IL-2, or (iv) PBS control. Additional cohorts were treated with (i) IL-2+anti-NK1.1 and (ii) IL-2+IL-33+anti-NK1.1. Additional cohorts were treated with (i) a combination of recombinant cytokines IL-2 and IL-25, (ii) recombinant IL-2, (iii) recombinant IL-25, (iv) recombinant IL-15, or (v) PBS control. Tumor size was monitored every two days until end point or when tumors reached 15 mm in diameter. Results are provided in FIG. 10.

Results in FIG. 10A show that B16F10 expression of cytokines (IL-2 and IL-33) promoted anti-tumor responses in a B16F10 melanoma model in Rag2−/− mice. Additionally, the antitumor effects of recombinant cytokines were diminished in the presence of antibodies for NK1.1, a marker for NK cells. These results indicate that NK cells are required for the anti-tumor effects of cytokine combination treatment. FIG. 10B shows that combining IL-2 with the Type 2 cytokine, IL-25, results in substantially improved inhibition of tumor growth.

### Example 6: In Vitro Expression of Type II Cytokine, IL-33, by TZ47 CART Cells

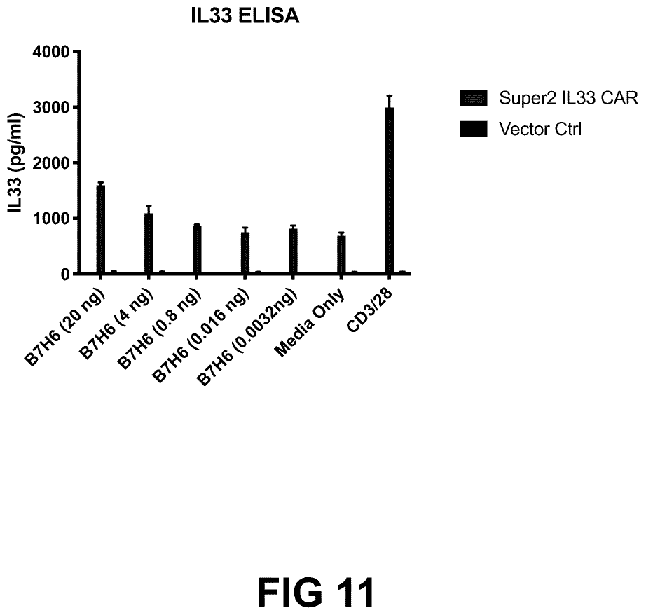

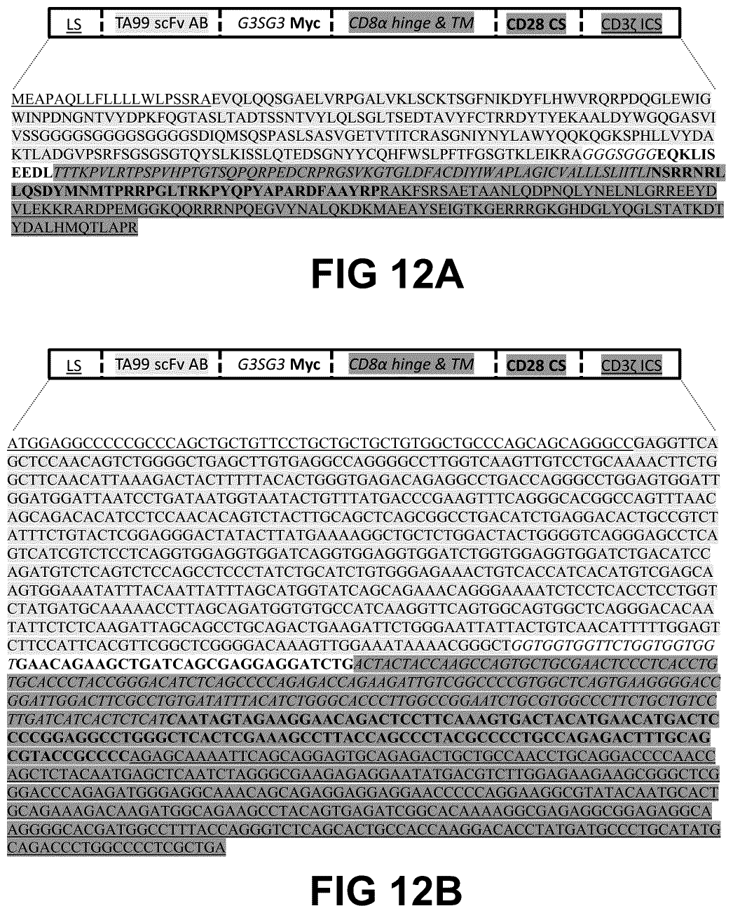

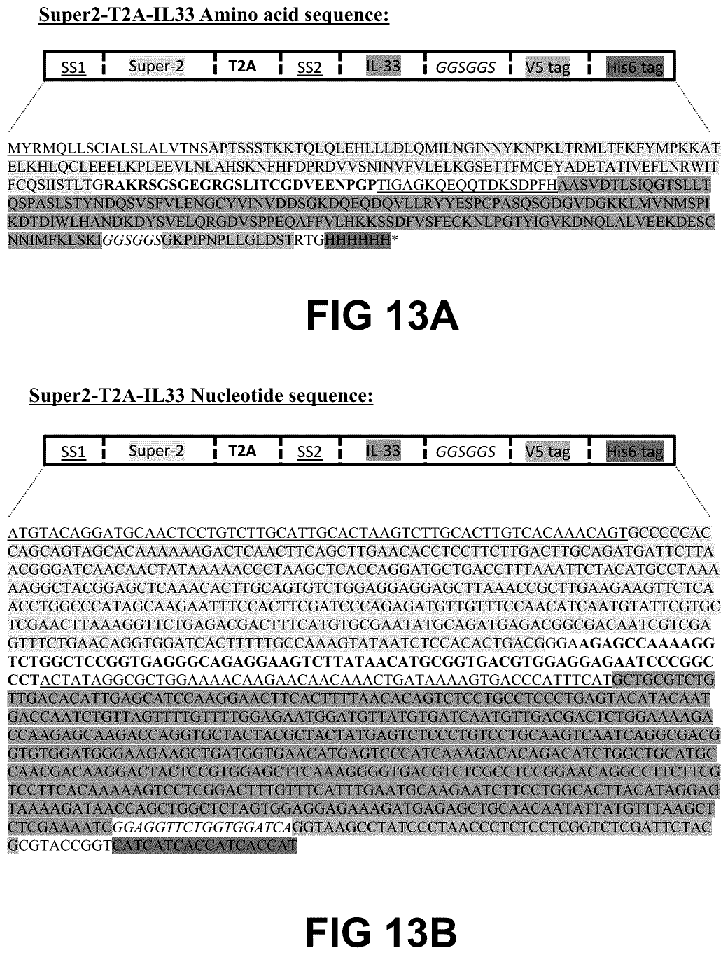

TZ47 CAR T cells specific for B7H6 were incubated with plate-bound recombinant hB7H6 (0 ng, 0.0032 ng, 0.0016 ng, 0.8 ng, 4 ng, or 20 ng) for 24 hours. IL-33 was assayed by ELISA in culture supernatants. Activated T cells transduced with empty vector backbone were used as a negative control. Stimulation using anti-CD3 and anti-CD28 antibodies was used as a positive control. Results are displayed in FIG. 11.

The graph in FIG. 11 shows a modest B7H6-dose dependent increase in IL-33 secretion. By contrast TCR stimulation using anti-CD3 and anti-CD28 antibodies promoted demonstrably higher IL-33 secretion. Control CAR T cells expressing an empty vector lacking Super-2-T2A-IL33 (vector control) did not express IL-33. These results indicate that CAR binding to B7H6 antigen is positively correlated with IL-33 expression. Because B7H6 is upregulated in solid and disseminated cancers, e.g. melanoma, lymphomas, etc., it can be reasoned that IL-33 secretion may likely be further increased by CAR association to B7H6 within a tumor microenvironment.

### Example 7: In Vitro Efficacy of CAR T Cells with Cytokine Constructs in Luminescent B16F10 Melanoma Cells

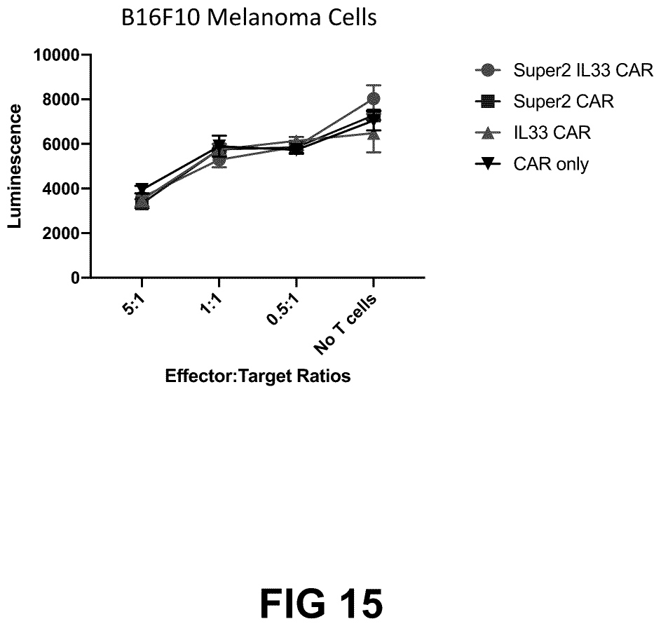

Luciferase-expressing B16F10 mouse melanoma (target) cells were cultured and treated with CAR T (effector) cells expressing (i) Super-2-T2A-IL-33 cytokine construct and TA99 CAR, (ii) Super-2 and TA99 CAR, (iii) IL-33 and TA99 CAR, and (iv) TA99 CAR only at effector: target cell ratios of 0.5:1, 1:1, and 5:1. Untreated luciferase expressing B16F10 cells were used as a negative control. Viability of B16F10 cells was monitored by luminescence activity on luciferin substrate. Results are displayed in FIG. 15.

The graph in FIG. 15 shows dose-dependent increase in cytotoxicity of all CAR T cells against B16F10 melanoma tumor cells. Although viability of cultured melanoma cells decreased with increasing dose of CAR T cells, no significant difference was observed in the cytotoxicity of said CAR T cells expressing an immunostimulatory combination of cytokines (Super-2 and Il-33), singly expressed cytokines, or no cytokine at all. These results indicate that expression of Super2, IL-33 or the combination of both Super-2 and IL-33 linked by T2A does not affect TA99 CAR T cell killing of luciferase-expressing B16F10 targets in vitro. It can be further reasoned that the synergistic effects of immunostimulatory cytokines on CAR T efficacy, such as those seen in FIGS. 7-10, are highly dependent on conditions within the tumor microenvironment.

### Example 8: In Vitro Cytotoxicity Assay

Natural killer (IL2Ra−/− NK1.1) cells were cultured with IL-2 and type 2 innate lymphoid cells (wt ILC2) or IL2Ra−/− ILC2 cells and stained with different stains. The cells were then sorted by FACS and the results are displayed in FIG. 16A-B.

FIG. 16A shows representative images of sorted cells with staining for the innate NK cell marker, (NK1.1, green), IL-2 receptor alpha chain (IL2Ra, CD25, red), cell trace violet (violet), and image overlays. FIG. 16B shows flow results of cytometry analysis of cultured cells based on Granzyme C (GzmC) and cell trace violet (CTV) staining. These results indicate that ILC2 cells may act as helpers that transfer CD25 (red staining in FIG. 16A) to NK cells (green staining in FIG. 16A), and presumably T cells, resulting in improved expression of effector granzymes as seen in FIG. 16B (lower left panel).

### Example 9: Immunohistochemical Staining of B16F10 Tumors of Expressing IL-2 Resected from C57BL/6 Mice

C57BL/6 mice were subjected to subcutaneous inoculation with 1×10\hat{ }6 B16F10 tumor cells. Tumors of B16F10 expressing IL-2 were resected 7 days after inoculation. Tumor sections were stained with antibodies against Thy1 (red), CD3 (cyan), and NK1.1 (green). Results are displayed in FIG. 17.

FIG. 17 shows representative images of immunohistochemical staining of an ILC2 cell (NK1.1−Thy1+CD3−) interacting with a T cell (NK1.1−Thy1+CD3+) and an NK or ILC1 cell (NK1.1+ Thy1+CD3−). Scale bar: 5 μm. The results of FIG. 17 support the application's statement that cell-cell interactions occur in B16F10 tumors between ILC2 helper cells (IL-33 responders) with T cells and NK cells (IL-2 responders).

### Example 10: Survival Differences in Endometrial Cancer Patient Populations

TCGA (Cancer Genome Atlas) patient data was graphed to show (i) immunohistochemical detection of B7H6 expression in various solid and disseminated cancer types and (ii) survival differences in endometrial cancer patients expressing higher IL-33 transcripts or a combination of IL-2 and CD8 T cells and ILC2-associated transcripts. Results are displayed in FIGS. 14 and 18, respectively.

FIG. 18 displays graphs showing percent survival over time for patients with endometrial cancers created with data from Human Protein Atlas according to Example. FIG. 18 (top) shows survival differences in endometrial cancer patients expressing higher IL-33 transcripts and those with low IL-33 expression. FIG. 18 (bottom) shows survival differences in endometrial cancer patients expressing a combination of IL-2 and CD8 T cells and ILC2-associated transcripts. The results of FIG. 18 indicate that higher IL-33 expression or a combination of higher CD8, IL-2 and ILC2 expression is correlated with better long-term survival.

### Example 11: In Vivo Effects of Cytokine Expressing CAR T Cells on B16F10 Tumors

B16F10 melanoma cell lines were cultured in Complete RPMI 1640 media (RPMI-1640 supplemented with 10% heat-inactive fetal bovine serum, 10 mM HEPES, 0.1 mM non-essential amino acids, 1 mM sodium pyruvate, 100 U/mL penicillin, 100 μg/mL streptomycin and 50 μM of 2-mercaptoethanol).

C57BL/6 mice (male and female, 8-14 weeks old) were subjected to intradermal (i.d.) inoculation with 2×10\hat{ }5 B16F10 tumor cells in 50 μL of HBSS into the right flank. B16F10 tumors were established for six days. Mice were then treated with 7×10\hat{ }6-8×10\hat{ }6 mouse CAR T cells expressing a second-generation CAR, TA99 scFv-CD8TM-CD28-CD3zeta (hereafter referred to as TA99 CAR) and Super2 in combination with either IL4, IL5, IL25, TSLP (thymic stromal lymphopoietin), or IL33 by intravenous tail vein injection. The TA99 CAR targets tyrosinase-related protein 1, Tyrp1 that is expressed on melanocytes and melanoma cells.

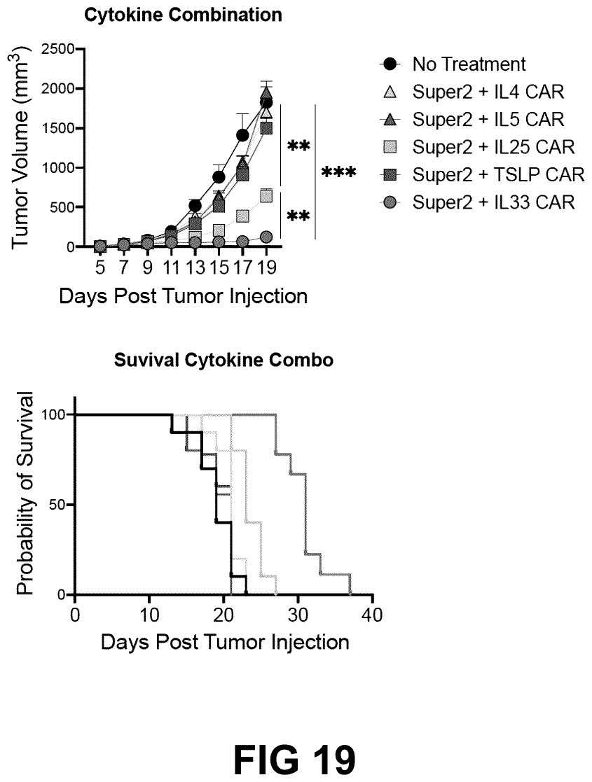

Tumor volume was monitored three times a week, and mice reaching maximum tumor burden (15 mm in diameter) or exhibiting moribund signs were euthanized. Each in vivo experiment was independently repeated at least twice. Results for tumor growth and percent survival are shown in FIG. 19.

Results indicate that TA99 CAR T cells expressing Super2+IL4, IL5, or TSLP have minor effects on tumor growth rate and survival compared to no treatment. Surprisingly, an increase in tumor growth inhibition and survival time was observed following treatment with Super2+IL25 TA99 CAR T cells and with Super2+IL33 CAR T cells. These results provide (i) initial proof of concept that co-expression of Super-2 and IL25 by TA99 CAR T cells provides synergistic enhancement of tumor control and (ii) further proof of concept that co-expression of Super-2 and IL-33 by TA99 CAR T cells provides synergistic enhancement of tumor control compared to other, less effective interleukin constructs (e.g., IL4, IL5, or TSLP).

### Example 12: Effects of Impaired TA99 CAR T Cell Therapy on B16-F10 Tumor Growth

T cell mediated killing of tumor cells is dependent on both perforin/granzyme B and IFNγ dependent pathways. To determine whether CAR T cell intrinsic expression of perforin or IFNγ is required for tumor control, TA99 CAR T cells expressing Super2 and IL-33 were engineered from perforin-deficient (Prf1 KO, Jax stock #002407) or IFNγ-deficient (IFNγ KO, Jax stock #002287) donor mice as detailed in Example 1.

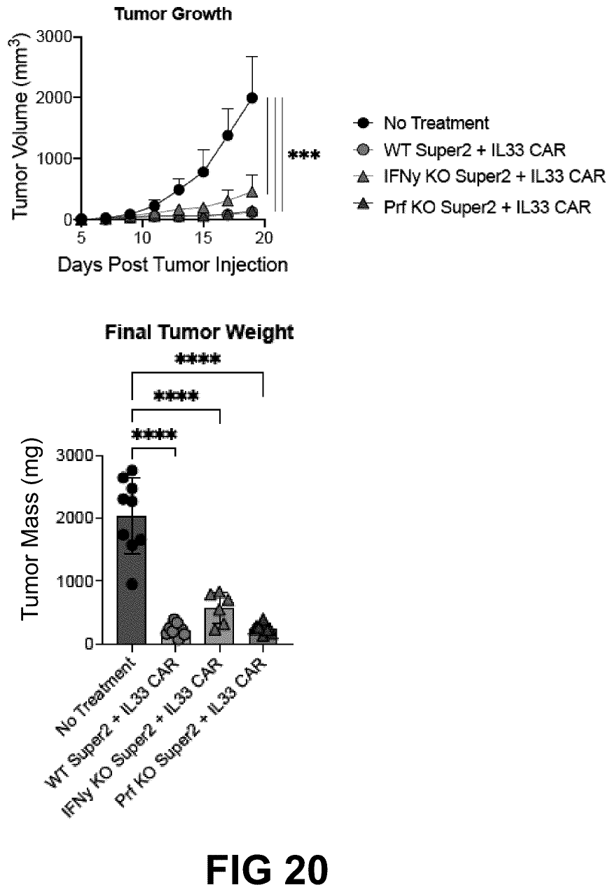

C57BL/6 mice were inoculated with B16F10 melanoma tumor cells and then treated with ˜7×10\hat{ }6 WT, Prf1-deficient, or IFNγ-deficient Super2+IL-33 TA99 CAR T cells as described in Example 11. Tumor volume was monitored every other day and final tumor masses were determined following removal on day 17 or 19 post inoculation when control tumors reached a maximum diameter of 15 mm. Results are shown in FIG. 20.

Results surprisingly indicate that mice treated with either Prf1-deficient or IFNγ-deficient TA99 CAR T cells expressing Super2 and IL-33 were similarly effective at controlling tumor growth compared to WT Super2+IL-33 TA99 CAR T cells. These data demonstrate that TA99 CAR expression contributes in part to anti-tumor responses and, surprisingly, CAR T cell-mediated tumor cytotoxicity through perforin/granzyme and IFNγ-dependent mechanisms were not required. This provides proof of concept that CAR T cells may be acting as a delivery vehicle for Super2 and IL-33, which synergistically activate endogenous immune responses that are largely responsible for tumor cytotoxicity.

### Example 13: Effects of Super2+IL33 T Cells without CAR Expression on B16F10 Tumor Growth

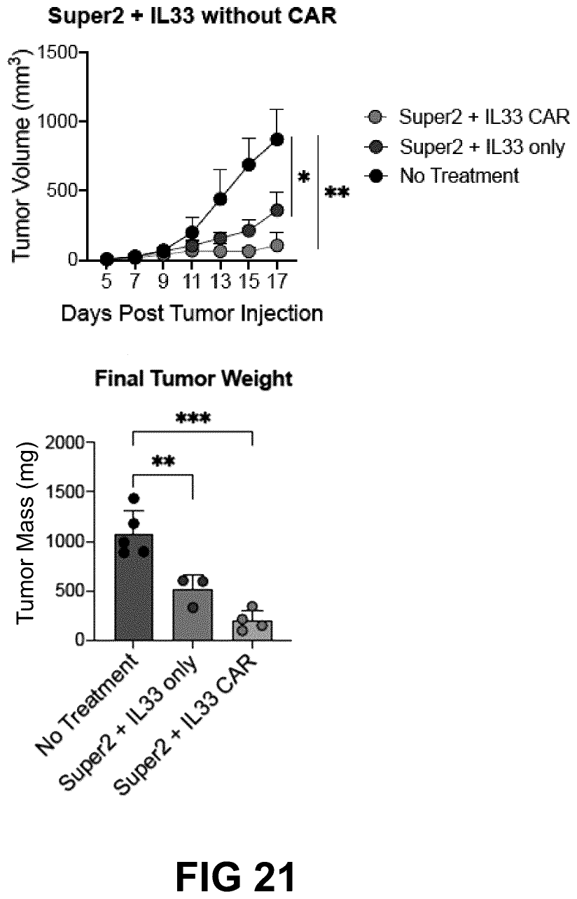

T cells expressing Super2 and IL33 without CAR expression were prepared according to Example 1. C57BL/6 mice were inoculated with B16F10 melanoma tumor cells and then treated with ˜7×10\hat{ }6 Super2+IL33 TA99 CAR T cells or Super2+IL-33 T cells as described in Example 11. Tumor volume was monitored every other day and final tumor masses were determined following removal on day 17 or 19 post inoculation when control tumors reached a maximum diameter of 15 mm. Results are shown in FIG. 21.

Results indicate that T cells expressing Super2 and IL-33 cytokines, but lacking TA99 CAR retained partial ability to control tumor growth, albeit at reduced efficiency compared to T cells expressing TA99 CAR and Super2+IL33. This provides further proof of concept that T cells act as a delivery vehicle for Super2 and IL-33, which synergistically activate endogenous immune responses, and that addition of a targeting moiety (e.g., TA99 CAR) which binds Tyrp1 in the solid tumors further enhances therapeutic effects.

### Example 14: Ex Vivo Analysis of B16F10 Tumor Infiltration by Leukocytes and Super2+IL33 TA99 CAR T Cells

Without being bound to any particular theory, it is thought that release of Super2 and IL-33 by CAR T cells into the tumor microenvironment activates both CAR T cells and endogenous immune cells. To distinguish the effects of Super2 and IL-33 on exogenous CAR T cells from the endogenous immune response, TA99 CAR T cells derived from CD45.1 congenically-marked cells were prepared and engineered to express Super2+IL33 according to Example 1. C57BL/6 mice were inoculated with B16F10 melanoma tumor cells and then treated with ˜7×10\hat{ }6 TA99 CAR T cells derived from CD45.1 with and without Super2+IL33 expression as described in Example 11.

Fifteen days post inoculation, tumor infiltrating leukocytes (TILs) were isolated from non-treated tumors, or tumors treated with TA99 CAR T cells alone or Super2+IL-33 TA99 CAR T cells. Tumors were extracted from mice and filtered through a wet 70 μm cell strainer (Falcon) and resuspended in RPMI media at 4° C. To isolate tumor infiltrating leukocytes, tumors were digested and resuspended in 80% percoll and overlayed by 40% percoll to create a separation gradient. Samples were spun at 350×g for 25 mins with no break. Buffy coat layer between 40% and 80% percoll was harvested and washed in PBS before further analysis and flow staining.

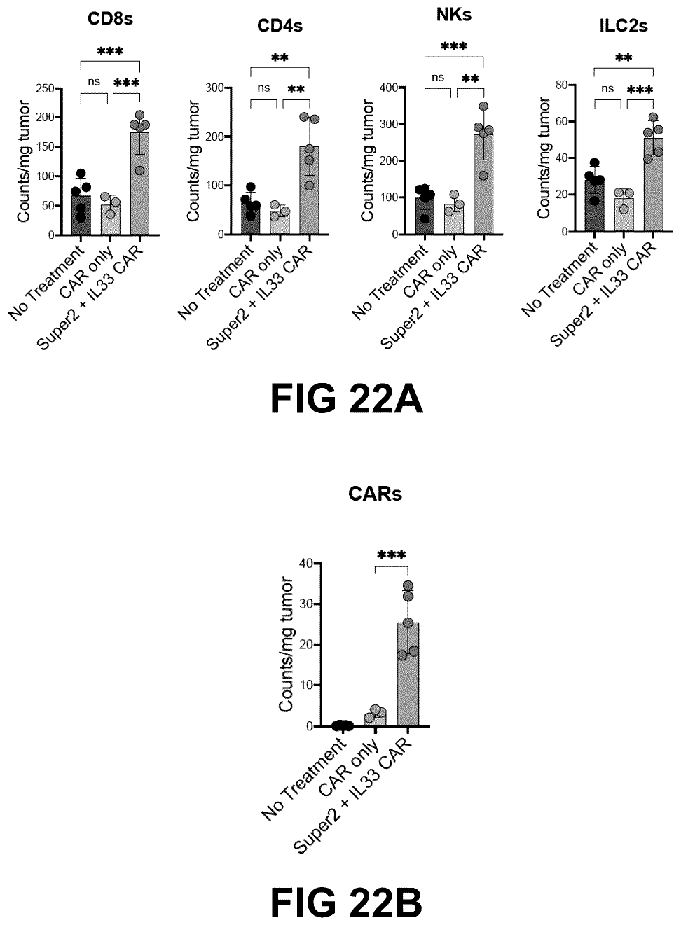

Tumor infiltrating leukocytes (TILs) were characterized by flow cytometry following staining with antibodies specific for CD8 T cells, CD4 T cells, NK cells, ILC2 cells, and CAR T cells. Flow cytometry analysis was performed on an Accuri C6 Cytometer (BD Biosciences) and data was analyzed using FlowJo software to determine TIL count normalized per gram of tumor. Results are shown in FIG. 22.

Results indicate that CAR T expression of Super2 and IL33 recruit and/or expand both endogenous TIL response and CAR T cell infiltration. Few TA99 CAR T cells (<5 CAR T cells/mg tumor) were isolated from TA99 CAR T cell treated mice, and the numbers of endogenous T cells and NK cell did not differ from non-treated mice. By contrast, significant increases in TA99 CAR T cells expressing Super2 and IL-33 observed as a result of increased CAR T cell infiltration, sustained survival, and/or expansion within the tumor. Endogenous CD8 and CD4 T cells, NK cells, and ILC2 cells were also significantly increased in number in TILs isolated from mice treated with TA99 CAR T cells expressing Super2 and IL-33 compared with TA99 CAR T cell treated or non-treated mice. In fact, endogenous tumor-infiltrating CD4 and CD8 T cells each outnumbered TA99 CAR T cells by ˜7-fold, and NK cells outnumbered TA99 CAR T cells ˜10-fold. These results indicate that CAR T cells expressing Super2 and IL-33 increased both CAR T cells as well as endogenous lymphocytes within the tumor, providing support for the proposed mechanism of release of Super2 and IL-33 by CAR T cells into the tumor microenvironment activates both CAR T cells and endogenous immune cells.

### Example 15: In Vivo Effects of Super2+IL33 CAR T Cells in Immune Depleted Mice

To determine which endogenous immune cell types (NK, CD4 T, CD8 and ILC2 cells) contribute to Super2+IL-33 CAR T cell-induced anti-tumor response. Each population of endogenous immune cells was individually depleted with antibody or genetically disrupted to determine their effect on antitumor immunity.

CD8-deficient or RORα-deficient mice were used as models of CD8 T cell and ILC2 cell deficiency, respectively. CD8-deficient (CD8 KO, Jax stock #002665) and RORα-deficient (RORα KO, Jax stock #005047) mice (male and female) were purchased from Jackson Laboratories and bred alongside C57BL/6 mice. NK cells and CD4 cells were depleted from C57BL/6 mice using either anti-NK1.1 or anti-CD4 antibodies, respectively. Depleting antibodies, anti-CD4 (mAb clone GK1.5) and anti-NK1.1 (mAb clone PK136; BioXCell), were administered via i.p. injection to C57BL/6 mice at 250 ug/dose every 3-4 days.

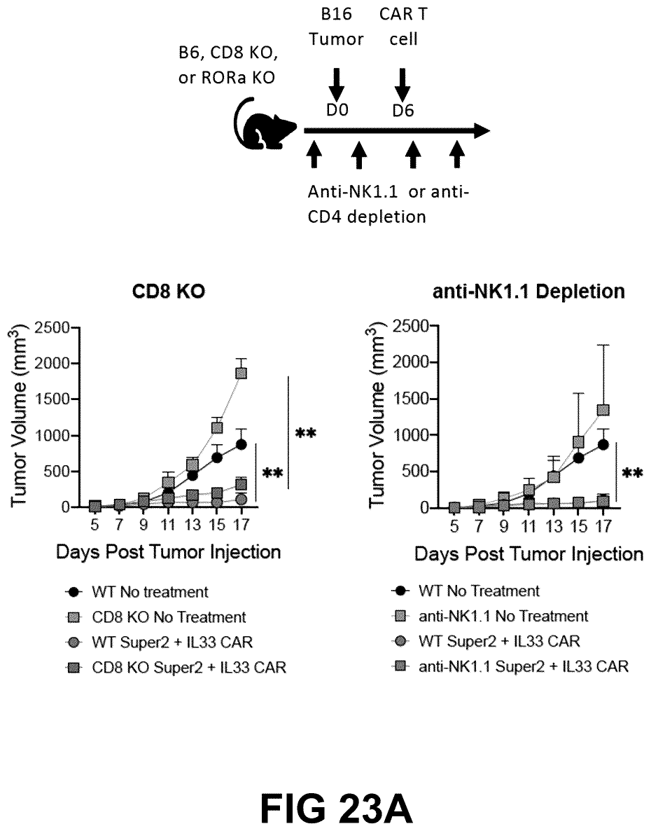

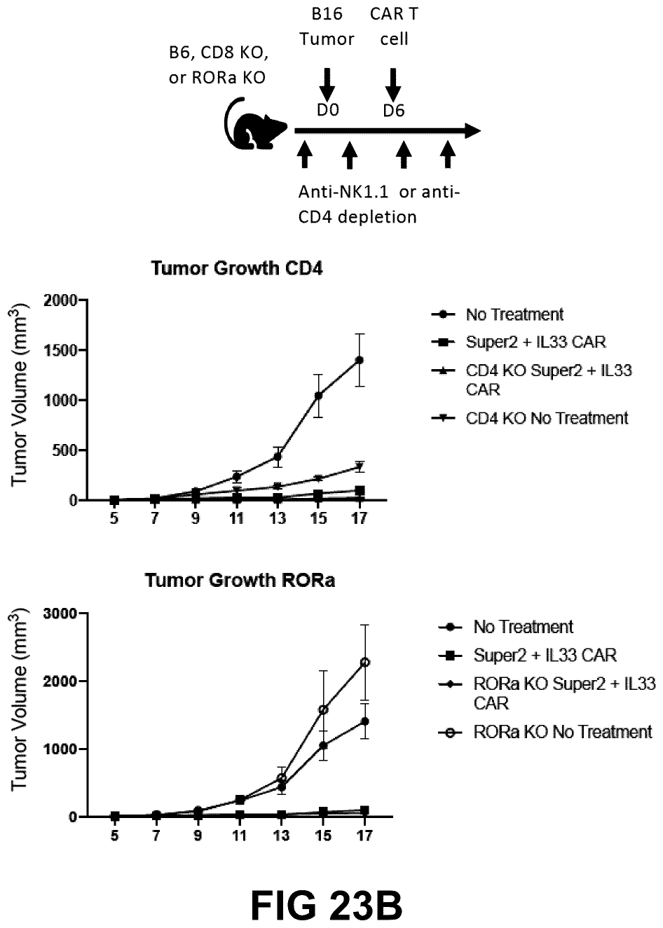

All mice (8-14 weeks old) were inoculated with B16F10 melanoma tumor cells and then treated with ˜7×10\hat{ }6 Super2+IL-33 TA99 CAR T cells as described in Example 11. Tumor volume was monitored every other day. Results of tumor growth over time are shown in FIG. 23.

Results indicate that CD4 depletion led to reduced tumor growth in non-treated mice. By contrast, non-treated tumors grew more quickly in NK-depleted, CD8-deficient, and ILC2-deficient mice compared to WT mice, indicating that these cytotoxic cells contribute to baseline antitumor responses. Across all mouse models, Super2+IL-33 TA99 CAR T cells induced comparably efficient anti-tumor immunity and decreased tumor growth compared to WT mice. Together, these data suggest that CAR T expression of Super2 and IL-33 does not rely on any one immune cell type but rather, the combination of Super2 and IL33 activates compensatory immune populations to decrease tumor burden.

### Example 16: Single Cell RNA Sequencing of B16F10 Tumor Infiltrating Leukocyte Populations Following Treatment with Super2+IL33 TA99 CAR T Cells

To systematically identify changes to the TIL populations within B16F10 tumors, single cell RNA sequencing (scRNAseq) analysis was performed on tumor infiltrating cells purified from non-treated, TA99 CAR T cell treated, and Super2+IL-33 TA99 CAR T cell treated mice. C57BL/6 mice were inoculated with B16F10 melanoma tumor cells and then treated with ˜7×10\hat{ }6 Super2+IL-33 TA99 CAR T cells as described in Example 11. B16F10 tumors were harvested 15 days post tumor inoculation, weighed, and then filtered through a wet 70 μm cell strainer (Falcon) and resuspended in RPMI media at 4° C. for scRNAseq analysis.

For scRNAseq analysis, cells were aggregated and clustered using Seurat nearest neighbor unsupervised clustering and visualized using Uniform Manifold Approximation and Projection (UMAP). Cluster cell identities were determined using the most upregulated genes in each cluster using VAM.

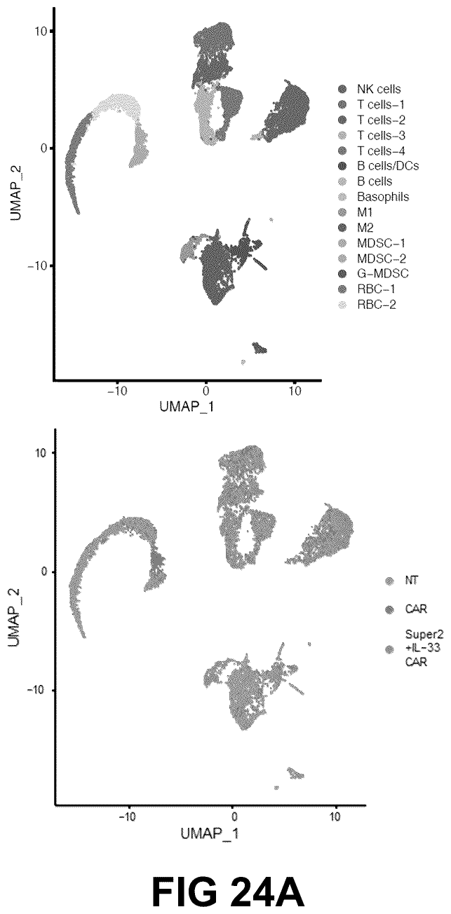

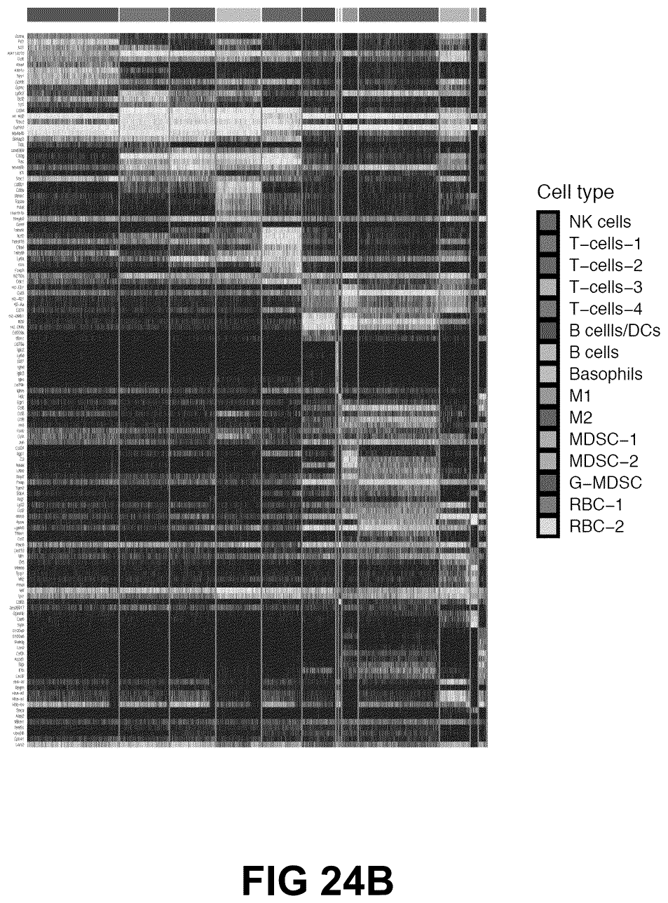

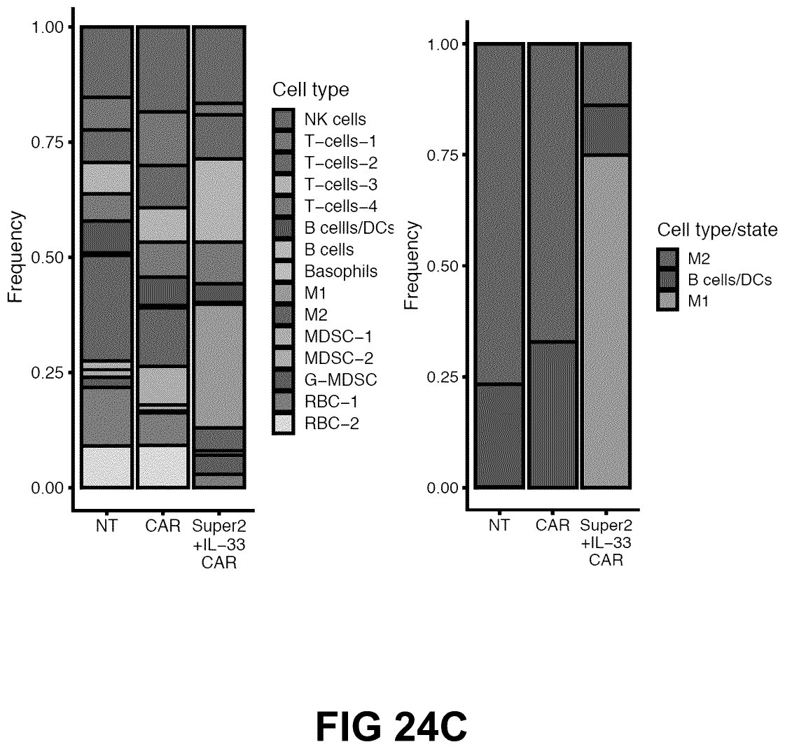

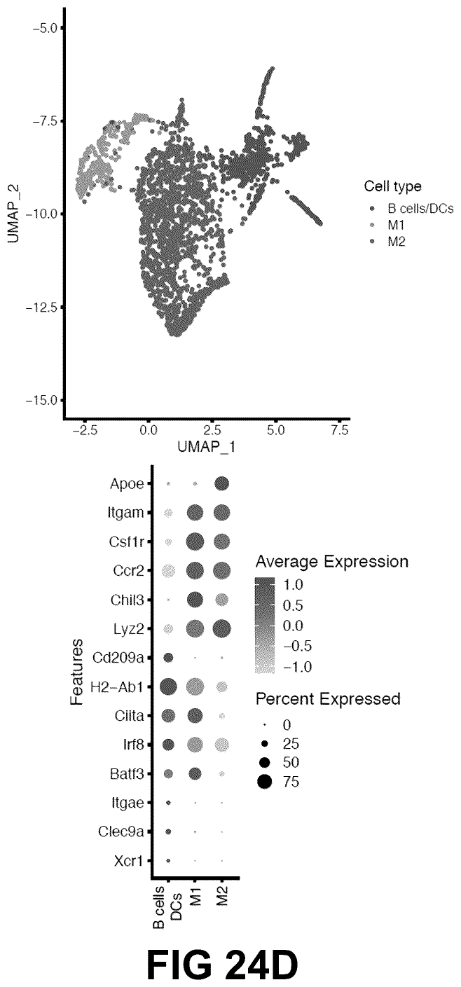

A total of 9767 cells were analyzed: 3151 cells from parental B16F10 mice, 5569 cells from the TA99 CAR T cell treated mice, and 1047 cells from the Super2+IL33 TA99 CAR T cell treated mice, for a total of 15295 genes. The transcript counts for these genes were then analyzed using Seurat v.4.0.1 in R v.4.0.2. Cells where mitochondrial genes made up more than 5% of total reads were removed, as were cells that had positive read counts fewer than 100 genes or greater than 3000 genes. Genes present in fewer than 10 cells were removed from the analysis. The cells and genes that remained were then normalized and transformed using SCTransform. Following transformation, nearest neighbor unsupervised clustering was performed using Seurat with a cluster resolution of 0.25. Visualization of these clusters was carried out using dimensionality reduction with Uniform Manifold Approximation and Projection (UMAP) on the first 30 principal components of the gene expression data. Differential gene expression was performed across the Seurat-determined clusters using a Wilcoxon Rank Sum Test. The most upregulated genes in each cluster were then leveraged to manually determine cluster cell types. The clusters identified as T-cells were isolated and TILPRED v.1.0.2 was performed to identify specific T-cell states. Results of scRNAseq analysis are shown in FIG. 24.

Data was visualized by generating 2-D UMAP plots showing (i) clustering of overall TIL populations differentiated by cell type and (ii) cell clustering differentiated by CAR treated tumors vs untreated tumors (FIG. 24A). A heatmap of gene expression in each TIL cell type cluster was generated (FIG. 24B). Graphs of frequency for each TIL cell type and frequency of monocytes, dendritic cells, and B cells for each treatment condition were generated (FIG. 24C). Graphs of cell clustering and gene expression values for monocytes, dendritic cells, and B cells within the tumor were generated (FIG. 24D).

Results indicate that few CAR T cells were identified in TIL populations compared to endogenous leukocytes, consistent with the flow cytometric TIL analysis. Comparison of endogenous TIL populations showed that similar immune cell types were present in all settings; however, proportional shifts in CD8 T cells, macrophages and MDSCs were observed. T cell state analysis with TILPRED v.1.0.2 identified shifts in CD8 effector, memory and exhausted populations. Macrophages shifted from M2-like to M1-like with decreased expression of ApoE and increased expression of antigen presentation machinery along with reduced expression of VEGF and IL-10.

Taken together, these results demonstrate how the tumor microenvironment shifts from immune suppressive to immune stimulatory to suppress tumor growth following treatment by CAR T cells that express Super2 and IL-33. This supports the hypothesis that Super2+IL-33 TA99 CAR T therapy alters TIL populations by recruiting or expanding several subsets of cells that together aid in the anti-tumor response.

### Example 17: Effects of Super2+IL33 CAR T Therapy on MC38 Colon Tumors and B16F10 Tumors Expressing B7H6

To demonstrate that the cytokine combinations and CAR platform of the present invention can provoke anti-tumor responses in other solid tumors independent of CAR target or tumor type, additional tumor models (e.g., MC38 colon tumors and B16F10 tumors expressing B7H6) were established.

MC38 colon tumor cells lines were purchased from ATCC and cultured in Complete DMEM media with a high glucose concentration (4.5 g/L) and supplemented with Complete RPMI 1640 media with the exception of 50 μM of 2-ME. For the MC38 mouse model, 1×10\hat{ }6 MC38 cells in 200 μL of HBSS were injected into the right flank of shaved C57Bl/6 mice via subcutaneous (s.c.) injection. Tumors were established for 6 days and then treated with 7-8×10\hat{ }6 mouse NKG2D CAR T cells or Super2+IL33 NKG2D CAR T cells via i.v. tail vein injections. The NKG2D CAR targets Rael, a stress ligand that is expressed naturally on MC38 colon tumors.

B16F10 melanoma cells were transduced with a dualtropic virus containing the human B7H6 gene. The cell line underwent Puromycin selection at a concentration of 2 μg/mL for approximately 5 days. Cells were then inoculated into C57Bl/6 mice and tumors cell lines were harvested and preserved post isolation from mice, providing a source of B16F10 cells with ectopic expression of the human tumor antigen B7H6. In this model, B16F10 tumor cells express heterogenous levels of B7H6 and lose B7H6 tumor antigen over time following treatment with T cells expressing TZ47, a B7H6-specific scFv CAR.

For the B7H6 tumor antigen model, 2×10\hat{ }5 B16F10 cells expressing B7H6 in 50 μL of HBSS were injected i.d. into the right flank of shaved C57Bl/6 mice. Tumors were established for 6 days and then treated with 4×10\hat{ }6 mouse TZ47 CAR T cells and Super2+IL33 TZ47 CAR T cells via intravenous tail vein injections.

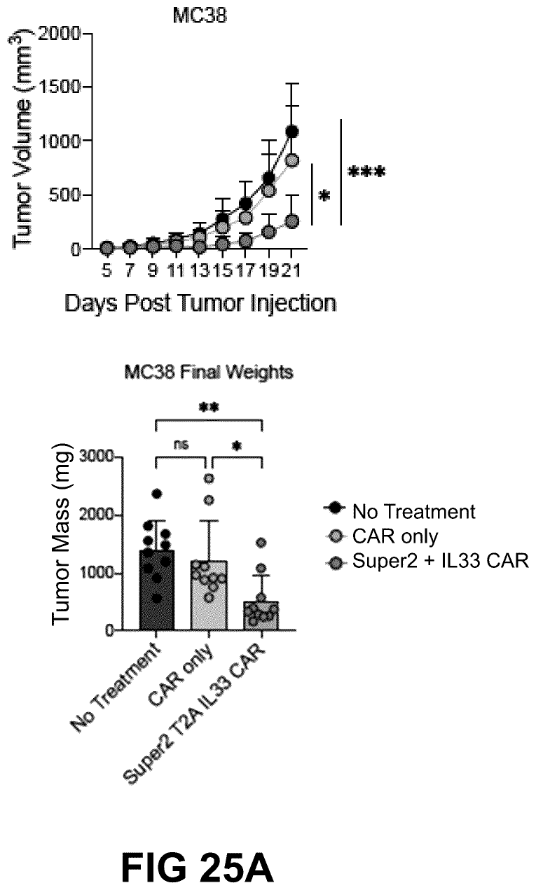

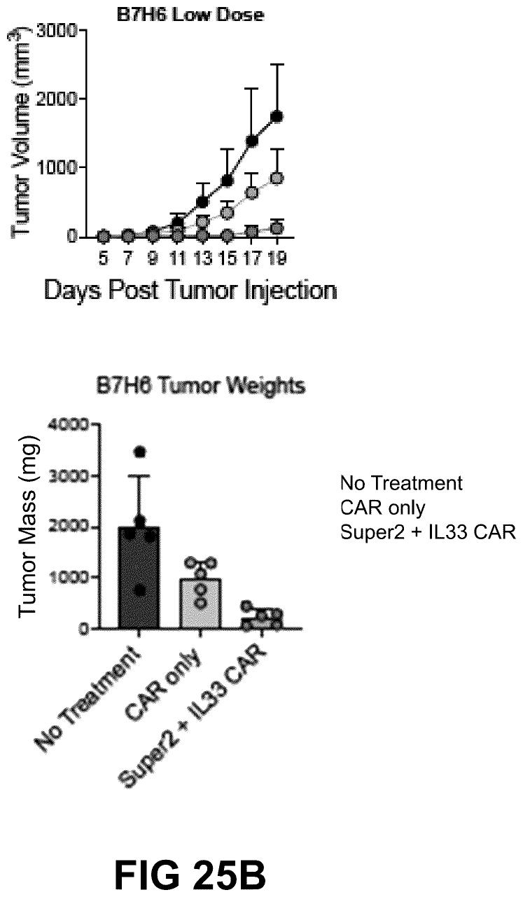

Tumor growth was monitored and final tumor weights of MC38 and B7H6 tumors were determined 21 and 19 days after inoculation, respectively. Results are shown in FIG. 25.

Results indicate a significant decrease in MC38 tumor growth in mice treated with NKG2D CAR T cells expressing Super2 and IL-33 as compared to mice treated with NKG2D CAR T cells alone or non-treated mice. For the B7H6 tumor antigen model, despite heterogenous tumor population and continual tumor antigen loss, TZ47 CAR T cells expressing Super2 and IL33 were able to significantly control tumor growth as compared to other treatment groups.

Materials & Methods for Examples 18-23

All experiments were conducted using the following general materials and methods unless otherwise indicated.

DNA Constructs

pCigar retroviral backbone was used to generate the TA99 CAR, and all cytokine expressing constructs. Combination cytokine constructs expressed the Super2 sequence, a T2A self-cleaving peptide, and the type 2 cytokine. All cytokine constructs were followed by an IRES GFP to determine transduction efficiency. pSFG backbone was used for the TZ47 CAR construct which included the CAR sequence, a T2A, followed by a truncated mouse CD19 sequence in which all signaling components were removed to act as a marker for transduction.

Cell Lines

B16F10 (CRL-6475) and HEK 293T (CRL-1168) cells lines were purchased from ATCC and maintained at low passages. MC38 (HT-29) cell line was purchased from Kerafast. The cell line B16-F10-B7H6 cell line was transduced with a dualtropic virus containing the human B7H6 gene. The cell line underwent puromycin selection at a concentration of 2 μg mL⁻¹ for approximately 5 days. Cells were then inoculated into B6 mice and tumors cells were harvested and preserved post isolation from mice.

Cell Culture

B16F10 melanoma cell lines and primary T cells were cultured in Complete RPMI 1640 media (RPMI-1640 supplemented with 10% heat-inactive fetal bovine serum, 10 mM HEPES, 0.1 mM non-essential amino acids, 1 mM sodium pyruvate, 100 U mL⁻¹ penicillin, 100 μg mL⁻¹ streptomycin and 50 μM of 2-mercaptoethanol). Primary T cells were also cultured in 25 U/mL recombinant human IL-2. HEK293T and MC38 cells were cultured in complete Dulbecco's modified Eagle's media (DMEM) media with a high glucose concentration (4.5g L⁻¹) and supplemented as with Complete RPMI 1640 media with the exception of 50 μM of 2-mercaptoethanol.

Retroviral Packaging

To package retrovirus, 2.5×10⁶ HEK-293T cells were plated on a 10 cm dish in complete DMEM 18 hrs before transfection. To produce ecotropic virus, cells were transfected using the calcium phosphate transfection method with 10 μg of CAR or cytokine plasmid and 10 μg of pCL-Eco packaging plasmid. Media was replaced with fresh media approximately 8 hrs post calcium phosphate treatment. Viral supernatants were harvested 48 hrs post transfection and filtered through a 0.45 μM filter before immediate use or flash freezing and storing at −80° C.

Generation of CAR T cells

Splenocytes from B6 mice or congenically marked CD45.1 B6 mice were transduced 18-24 hrs after Concanavalin A (1 μg mL⁻¹; Sigma) stimulation and cultured in Complete RPMI media plus 25 U mL⁻¹ of IL-2. Mouse T cells were transduced with retrovirally encoded cytokine supernatant by resuspending activated T cells in viral supernatant and polybrene (1 ug mL⁻¹; Sigma) at 8 million cells per well in 24 well plates followed by spinoculation at 1500×g for 90 mins at 37° C. Approximately 24 hours later, cells were transduced a second time to express the CAR receptor as performed on the previous day. Two days post-infection, cells were analyzed by flow cytometry to detect CAR expression before experimental set up or CAR T administration to mice.

Cytotoxicity Assays

Luciferase-based Cytotoxicity: B16F10 tumor cells expressing click beetle green luciferase were plated at 5×10⁴ cells per well in a white 96-well flat-bottom plate. CAR T cells were added at various T cell effector to target ratios (E:T) of 5:1, 1:1, 0.5:1. Cells were co-cultured at 37° C. for 24 hrs followed by addition of 50 μl of luciferin (200 μg mL⁻¹; GoldBio) and incubated at 37° C. for 30 mins before detecting luminescence via a Centro LB960 Berthold Technologies luminometer.

In Vivo Mouse Experiments

Male and female mice were used for in vivo experiments. Tumor volume was monitored three times a week, and mice reaching maximum tumor burden (15 mm in diameter) or exhibiting moribund signs were euthanized. Each in vivo experiment was independently repeated at least twice.

For the B16 primary tumor model, 2×10⁵ B16F10 cells in 50 μl of HBSS were injected via intradermal (i.d.) injection into the right flank of shaved C57Bl/6 mice. Tumors were established in mice for 6, 11 or 14 days and then treated with 7×10⁶ mouse TA99 CAR T cells via intravenous (i.v.) tail vein injections. For the metastatic tumor model, 2×10⁵ B16F10 cells in 200 μl of HBSS were injected i.v. through the tail vein of C57Bl/6 mice. After the tumors established for 6 days, 7×10⁶ mouse TA99 CAR T cells were injected through the tail vein.

### Example 18: Efficacy of Super2+IL-33 CART Cells Against Medium and Large Tumors

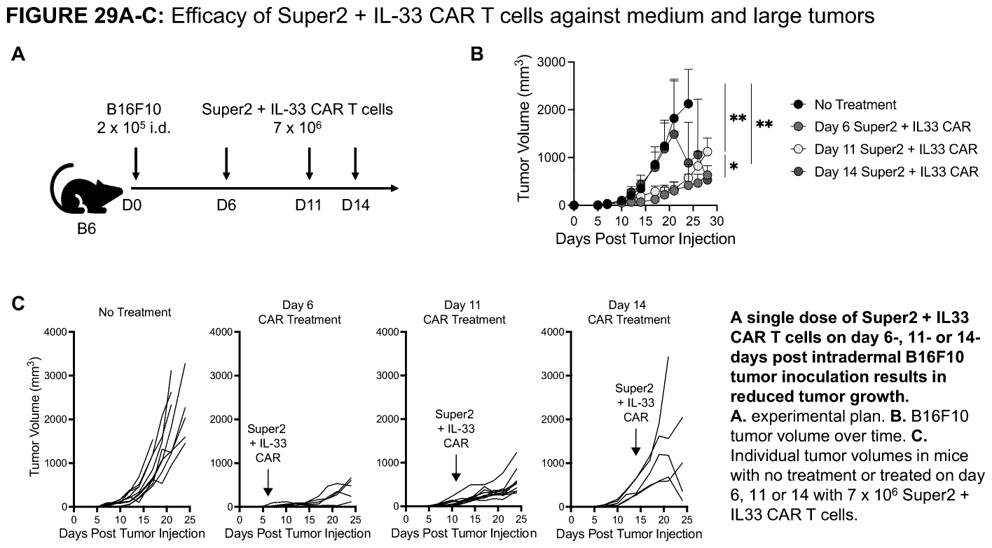

This example relates to the experiments in FIG. 29A-C. In these experiments a single dose of Super2+IL33 CAR T cells on day 6-, 11- or 14-days post intradermal B16F10 tumor inoculation results in reduced tumor growth. The results are shown in FIG. 29A-C. The sequences of full length TA99 CAR and Super2+IL-33 used in the experiments are the same as in the prior examples.

FIG. 29A contains a schematic of the experimental design showing B6 mice challenged with 2×10⁵ B16F10 tumors in the dermis were treated with 7×10⁶ Super2+IL-33 TA99 CAR T cells intravenously in the tail vein 6, 11 or 14 days post tumor inoculation. FIG. 29B shows the B16F10 tumor volume over 28 days. FIG. 29C shows individual tumor volumes in mice with no treatment or treated on day 6, 11 or 14 with 7×10⁶ Super2+IL33 CAR T cells. n=10 (NT), 5 (day 6), 11 (day 11), 5 (day 14).

The results demonstrate the efficacy of the combination of Super2+IL-33 CAR T cells against both different size tumors. In the experiments we conducted treatment at delayed time points to determine the ability of Super2/IL33 CAR T cells to limit larger tumors. We tested CAR T cell treatment on day 11 (average tumor size: 7.5 mm×6 mm) and day 14 (average tumor size: 10.5 mm×8 mm) in addition to our standard treatment on day 6 (average tumor size: 3 mm×2.5 mm). For mice treated with on day 6 or day 11, we observed a statistically significant delay in tumor growth 1 week after CAR T cell administration in all treated mice. For mice treated on day 14, we observed variable delay in tumor growth by day 24, with some mice showing CAR T cell efficacy while others did not. Thus, we found that Super2/IL33 CAR T cells are effective at treating medium size tumors (7.5 mm) but large tumors (10 mm) had variable responsiveness, with a portion of mice reaching a point of no return because of their tumor size.

### Example 19: CAR T Cells Expressing Super2+IL-33 have Improved Expansion and Remain in B16F10 Tumors Longer

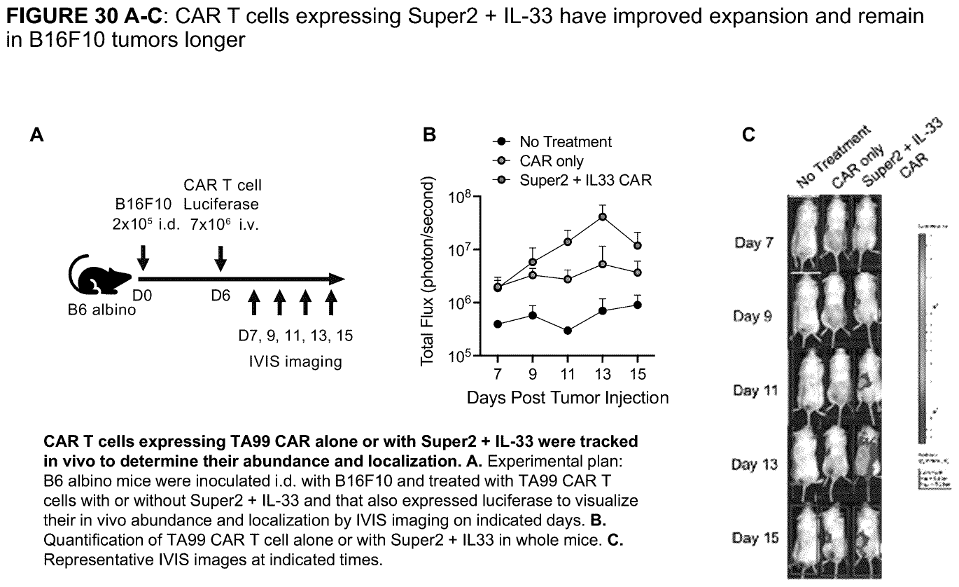

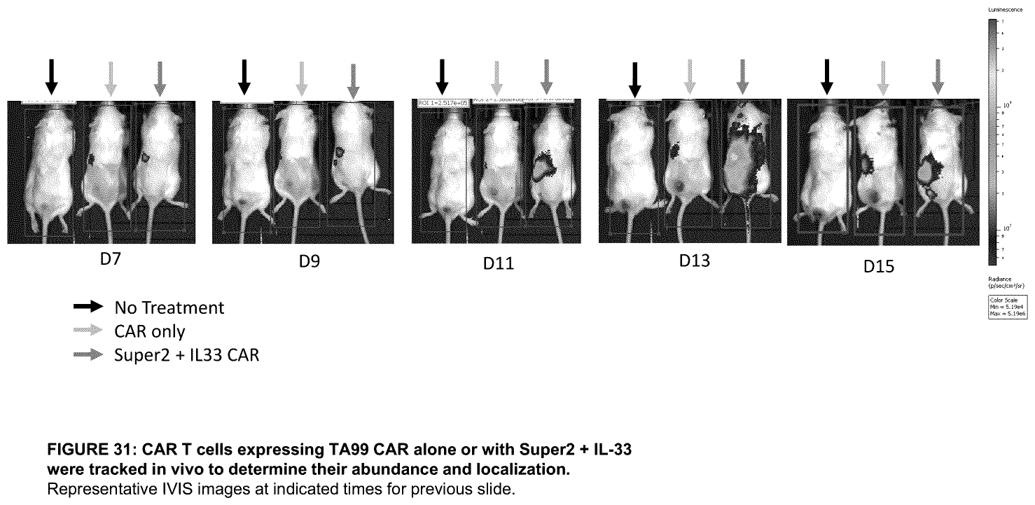

In these experiments CAR T cells expressing TA99 CAR alone or with Super2+IL-33 were tracked in vivo to determine their abundance and localization. The results are in FIG. 30A-C and FIG. 31. The results of the experiments show that single dose of Super2+IL33 CAR T cells on day 6-, 11- or 14-days post intradermal B16F10 tumor inoculation results in reduced tumor growth.

FIG. 30A contains an experimental schematic showing that B6 albino mice were inoculated i.d. with 2×10⁵ B16F10 tumors and treated on day 6 with 7×10⁶ TA99 CAR T cells with or without Super2+IL-33 and that also expressed luciferase to visualize their in vivo abundance and localization by IVIS imaging on indicated days.

FIG. 30B shows the quantification of TA99 CART cell alone or with Super2+IL33 in whole mice. n=4 (NT), 9 (TA99 CAR only), 10 (Super2+IL-33 TA99 CAR). FIG. 30C and FIG. 31 contain representative IVIS images at indicated times.

### Example 20: TA99 CAR Constructs Lacking Function CD28 and CD3 Zeta Signaling Motifs Retain Considerable Efficacy

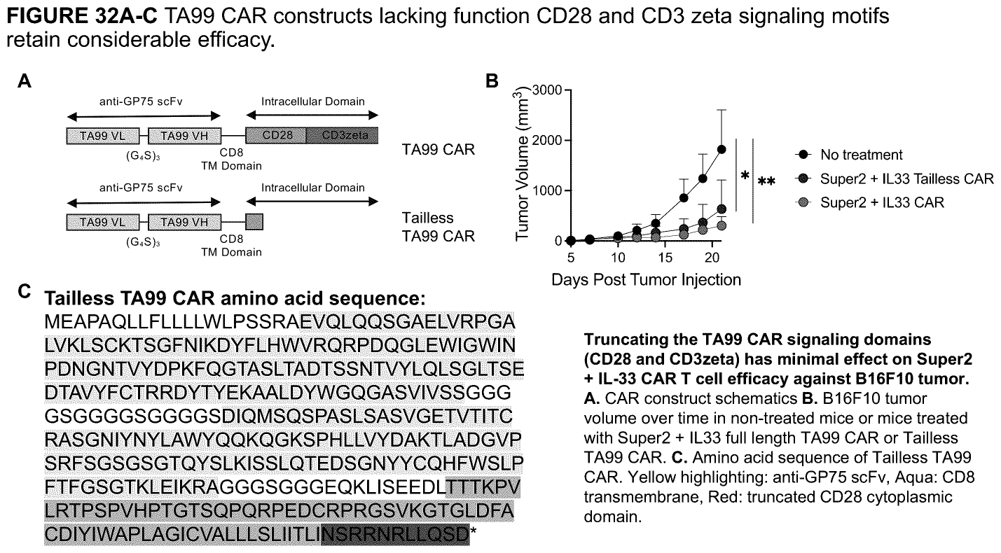

In these experiments the effects of truncating the TA99 CAR signaling domains (CD28 and CD3zeta) was assessed and demonstrated to elicit minimal effect on Super2+IL-33 CAR T cell efficacy against B16F10 tumors. These experimental results are in FIG. 32A-C.

The sequences of the Tailless CAR used in the experiments are set forth below:

A) Sequence Encoding Tailless TA99 CAR
[symbol] Signal sequence is underlined
[symbol] GGSGGS Linker is in italics
[symbol] Myc tag is bolded
[symbol] TA99 scFv is highlighted gray
[symbol] mouse CD8a partial extracellular and entire transmembrane domain, truncated mouse CD28 cytoplasmic domain are highlighted green

Tailless TA99 CAR Nucleotide sequence:

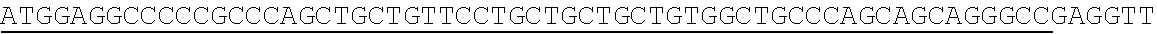

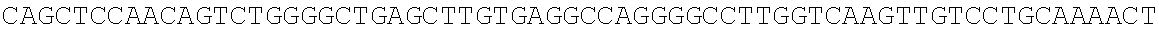

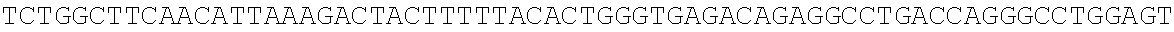

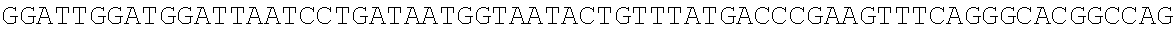

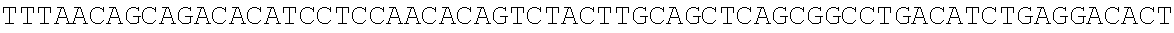

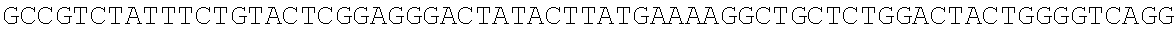

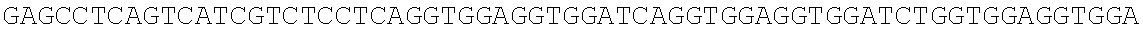

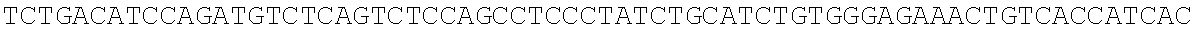

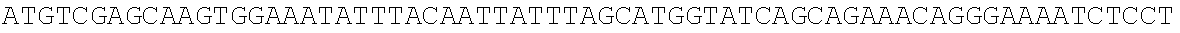

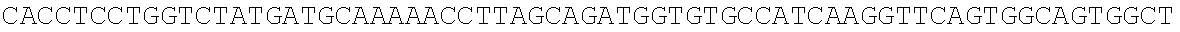

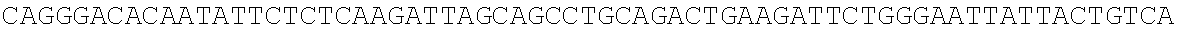

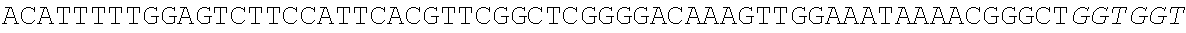

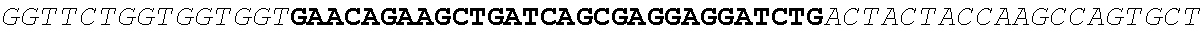

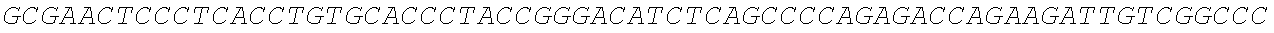

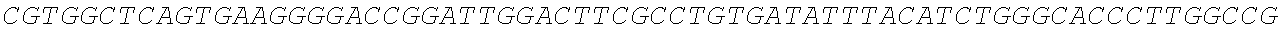

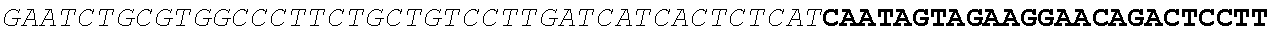

CAAAGTGACT
Tailless TA99 CAR Amino acid sequence:

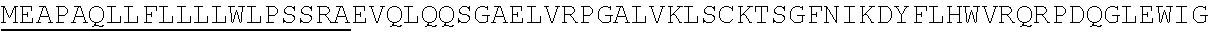

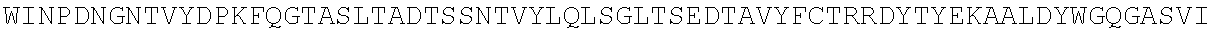

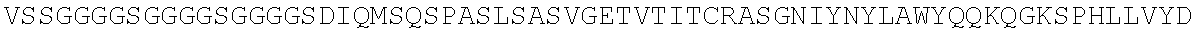

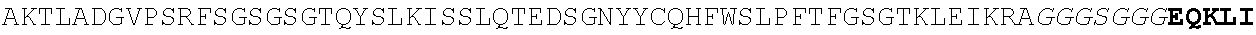

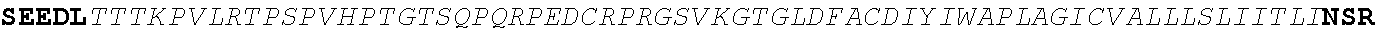

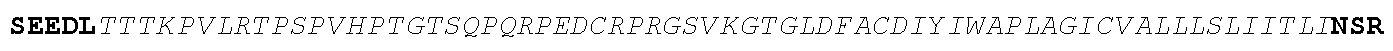

RNRLLQSD*

FIG. 32A contains a schematic of the CAR construct used in the experiment. FIG. 32B shows results wherein B6 mice challenged with 2×10⁵ B16F10 tumors in the dermis were treated with 7×10⁶ Super2+IL-33 full length or Tailless TA99 CAR T cells intravenously in the tail vein 6 days post tumor inoculation. Tumor volume was measured over 21 days. FIG. 32C contains the amino acid sequence of tailless TA99 CAR. Yellow highlighting: anti-GP75 scFv, Aqua: CD8 transmembrane, Red: truncated CD28 cytoplasmic domain.

The results show that truncating the TA99 CAR signaling domains (CD28 and CD3zeta) has minimal effect on Super2+IL-33 CAR T cell efficacy against B16F10 tumor. Similar to data where we demonstrated that CAR-less T cells expressing Super2+IL-33 show good efficacy, we believe that CAR expression is dispensable for activating endogenous tumor immunity. The T cells (or frankly any carrier, cell or non-cell) acts as a delivery vehicle for the cytokines. However it is noted that CAR expression is not irrelevant since CAR recognition of tumor antigen likely contributes to tumor infiltration and residence.

### Example 21 Super2+IL-33 TA99 CAR T Cell Treatment Induces Changes in Regulatory T Cells Gene Expression

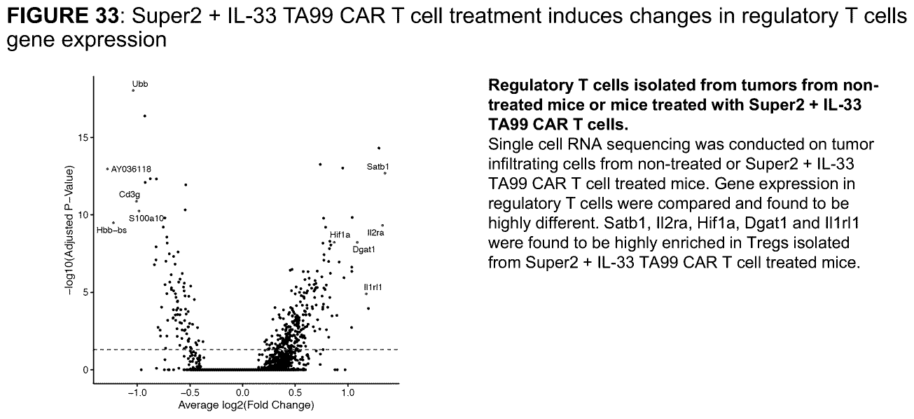

In this example the results of which are in FIG. 33 regulatory T cells were isolated from tumors from non-treated mice or mice treated with Super2+IL-33 TA99 CAR T cells. Tumor infiltrating cells were subjected to scRNA-seq using the 10× Genomics platform according to manufacturer's directions. Transcripts were aligned to the mm10 mouse reference genome; barcode processing and transcript quantification were performed on the Cell Ranger Single-Cell Software Suite (10× Genomics). A total of 9767 cells were analyzed: 3151 cells from parental B16F10 mice, 5569 cells from the TA99 CAR-treated mice, and 1047 cells from the TA99 CAR Super2-IL-33 T2A-treated mice, for a total of 15295 genes. The transcript counts for these genes were then analyzed using Seurat v.4.0.1 in R v.4.0.2. Cells where mitochondrial genes made up more than 5% of total reads were removed, as were cells that had positive read counts fewer than 100 genes or greater than 3000 genes. Genes present in fewer than 10 cells were removed from the analysis. The cells and genes that remained were then normalized and transformed using SCTransform. Following transformation, nearest neighbor unsupervised clustering was performed using Seurat with a cluster resolution of 0.25. Visualization of these clusters was carried out using dimensionality reduction with Uniform Manifold Approximation and Projection (UMAP) on the first 30 principal components of the gene expression data. Differential gene expression was performed across the Seurat-determined clusters using a Wilcoxon Rank Sum Test. The most upregulated genes in each cluster were then leveraged to manually determine cluster cell-types.

FIG. 33 contains the results of said single cell RNA sequencing conducted on tumor infiltrating cells from non-treated or Super2+IL-33 TA99 CAR T cell treated mice. The volcano plot in the Figure displays differential gene expression in regulatory T cells from non-treated vs Super2+IL-33 TA99 CAR T cell treated tumor infiltrating leukocytes (TILs). A total of 339 statistically significant differentially expressed genes (increased or decreased) was observed. As shown Satb1, Il2ra, Hif1a, Dgat1 and Il1rl1 were found to be highly enriched in Tregs isolated from Super2+IL-33 TA99 CAR T cell treated mice.

These results are compelling as Satb1 repression reportedly is required for Treg activity (See Beyer et al., Nat Immunol. 2011 “Repression of the genome organizer SATB1 in regulatory T cells is required for suppressive function and inhibition of effector differentiation”). Moreover, Dgat1 deficiency has been reported to be associated with higher Treg activity (See Graham et al., “DGAT1 inhibits retinol-dependent regulatory T cell formation and mediates autoimmune encephalomyelitis’, Proc Natl Acad Sci USA. 2019 Feb. 19; 116(8):3126-3135). Further, Il1rl1 expressing Tregs reportedly are associated with advanced tumors (see Li et al., “IL-33 Signaling Alters Regulatory T Cell Diversity in Support of Tumor Development”. Cell Rep., 2019 Dec. 3; 29(10):2998-3008.e8).

### Example 22: Human T Cells Expressing TZ47 (Anti-B7H6) CAR and are Equally Effective at Killing K562 Tumors Expressing B7H6 Tumor Antigen with or without Super2+IL-33 Expression

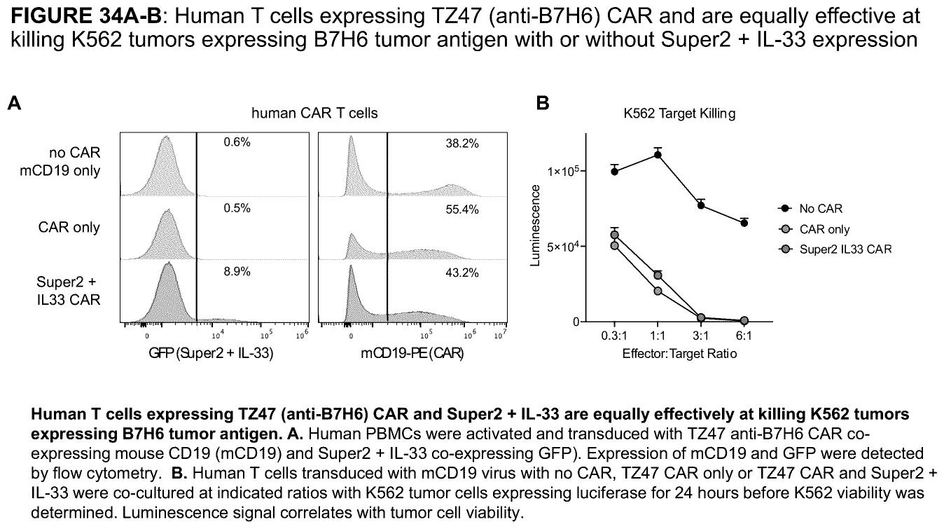

In the experiments in this example we showed that human T cells expressing TZ47 (anti-B7H6) CAR and Super2+IL-33 are equally effectively at killing K562 tumors expressing B7H6 tumor antigen. These results are in FIG. 34A-B.

In the experiment in FIG. 34A human PBMCs were stimulated for two days starting on with anti-CD3 (clone OKT3) and transduced on days 2 and 3 with PG13-packaged viruses (as described in original methods). The TZ47 anti-B7H6 CAR co-expressed mouse CD19 (mCD19) and Super2+IL-33 co-expressed GFP. On day 8, T cells were stained with antibodies specific for mCD19, hCD3, hCD8, mCD19, protein L (for CAR) and GFP and analyzed by flow cytometry, or as shown in FIG. 34B were co-cultured with B7H6-expressing K562-Luc for 24 hours. The cytotoxicity of K562 was measured using a luciferase cytotoxicity assay wherein the intensity of the luminescence signal correlates with tumor cell viability.

The results further demonstrate that human TZ47 CAR T cell with or without Super2+IL-33 co-expression were equally cytotoxic against tumor antigen-expressing targets in vitro, demonstrating that expression of these cytokines does not alter the activity of CAR T cells in the absence of a tumor microenvironment.

### Example 23: Expression of Super2+IL-33 does not Alter the Transcriptional Profile or Subset Distribution of Human TZ47 (Anti-B7H6) CAR T Cells

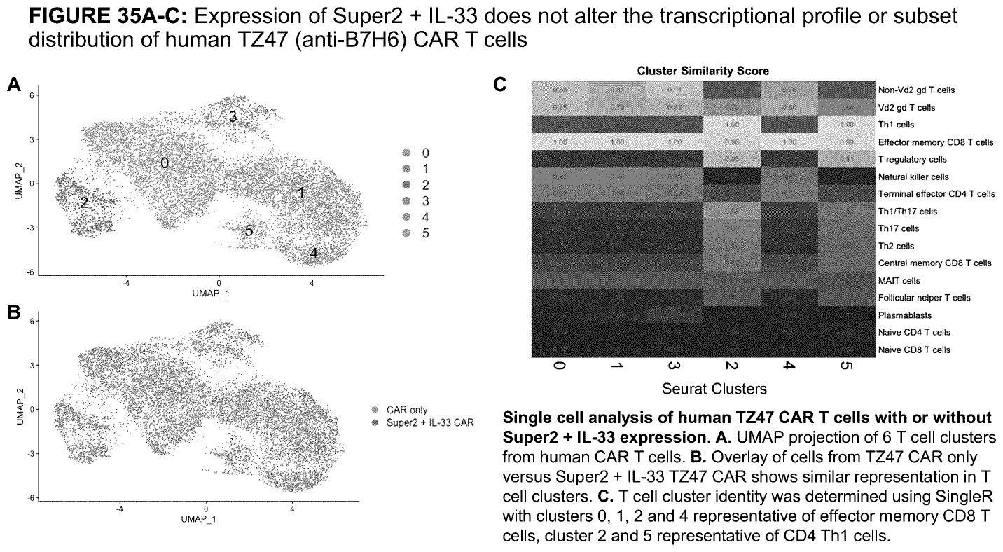

In this example we conducted a single cell analysis of human TZ47 CAR T cells with or without Super2+IL-33 expression. These experimental results are in FIG. 35A-C.

In these experiments the transcripts were aligned to a modified GRCh38 reference genome to include mCD19 and IRES-GFP for the detection of TZ47 CAR and Super2+IL-33 expression respectively. Count matrices for each library were filtered to exclude outliers. Cells with greater than 20% mitochondrial gene percentage, 7500 genes, or 30000 transcript reads were excluded, as were cells with fewer than 350 genes, or fewer than 1 transcript read. After filtering, 12196 cells remained for analysis: 5896 cells from CAR only and 6300 from Super2+IL-33 CAR. Clustering and visualization were performed as in general methods, with a clustering resolution of 0.22 and with 20 principle components. Reference-based cluster annotation was performed using the SingleR package with MonacolmmuneData as a reference for immune cell types (Aran D, Looney A P, Liu L, Wu E, Fong V, Hsu A, Chak S, Naikawadi R P, Wolters P J, Abate A R, Butte A J, Bhattacharya M (2019). “Reference-based analysis of lung single-cell sequencing reveals a transitional profibrotic macrophage.” Nat. Immunol., 20, 163-172. doi: 10.1038/s41590-018-0276-y; Monaco G et al. (2019). RNA-Seq Signatures Normalized by mRNA Abundance Allow Absolute Deconvolution of Human Immune Cell Types”, Cell Rep. 26, 1627-1640.

FIG. 35A shows human TZ47 CAR T cells prepared as described in the previous examples and subjected to single cell RNA sequencing as described above. The UMAP projection of 6 T cell clusters from human CAR T cells is shown therein. FIG. 35B shows an overlay of cells from TZ47 CAR only sample versus Super2+IL-33 TZ47 CAR sample and contains a similar representation in T cell clusters. FIG. 35C shows T cell cluster identity determined using SingleR (see methods above) with clusters 0, 1, 2 and 4 representative of effector memory CD8 T cells, cluster 2 and 5 representative of CD4 Th1 cells.

The expression of Super2 and IL-33 in human CAR T cells does not change the original CAR T cell product (e.g. alter proportions of effector and memory T cells) regardless of the CAR the cytokine platform is paired with. Accordingly the observed therapeutic benefits are likely because of the neutralization of the immunosuppressive tumor microenvironment which benefits will be seen in other tumors.

### CONCLUSIONS

Together, our experimental results demonstrate that a single dose of Super2+IL-33CAR T cells delays tumor growth and can drive complete remission in various models independent of the CAR-specific tumor target and tumor type. Additionally, CAR T cells expressing Super2 and IL-33 can more effectively control heterogenous tumors with the propensity to downregulate tumor antigen, consistent with the ability of Super2 and IL-33 to synergistically induce endogenous immune responses and shift immunosuppressive cells within the tumor microenvironment to an immunostimulatory phenotype. These findings show that engaging both type 1 and type 2 cytokines in addition to CAR T cell-mediated killing is an effective treatment strategy for combating solid cancers. Expression of Super2 and IL-33 with different CAR targeting constructs enabled effective control of multiple tumor types, indicating the potential of this approach as a universal therapeutic platform.

Moreover, our results indicate that the expression of Super2 and IL-33 in human CAR T cells will not change the original CAR T cell product (e.g. alter proportions of effector and memory T cells) regardless of the CAR the cytokine platform is paired with. This is because of our belief that the therapeutic benefits of this combination which were observed in vivo are likely obtained as a result of neutralization of the immunosuppressive tumor microenvironment.

The therapeutic efficacy of CAR T cells is predominantly determined in preclinical studies using immunodeficient mouse models such as NSG mice to allow engraftment of both human CAR T cells and their tumor targets. Yet these models poorly recapitulate the suppressive TME that determines whether CAR T cells ultimately control tumor growth. When immunocompetent models are used, preconditioning the host with irradiation, immunodepleting regimens or immunosuppressive chemotherapies to generate space for CAR T cells and to reduce immunosuppression is often necessary to drive CAR T cell expansion; however, therapeutic responsiveness remains limited. Thus, significant opportunity remains to develop approaches to overcome the daunting hurdle presented by the TME for CAR T cell therapy for solid cancers.

We show that Super2/IL-33 expressing CAR T cells mount a potent anti-tumor response in immunocompetent mice in the absence of pre-conditioning. In fact, general depletion of host immune cells could arguably impede the overall efficacy of Super2/IL-33 CAR T cells given the ability of these cells to activate a robust response from endogenous anti-tumor immune cells.

Indeed, lymphodepleting chemotherapies aimed at generating immunological space, i.e. excess homeostatic cytokines such as IL-2, may no longer be required with Super2 engineered CAR T cells. Future studies that directly evaluate the effects of lymphodepletion on Super2/IL-33 CAR T cell immunotherapy will help to further inform clinical translation.

Loss of tumor antigens due to tumor heterogeneity and immune editing is an additional hurdle limiting the efficacy of CAR T cell immunotherapy and other antigen-specific therapies. To examine the effect of antigen heterogeneity in the context of CAR T cell immunotherapy, we engineered T cells to express a B7H6-specific CAR, TZ47 and then treated mice that had been inoculated with a heterogeneous mix of parental B16F10 and B16F10 expressing the human tumor antigen B7H6. Preferential loss of B7H6-expressing tumor cells following TZ47 CAR T cell treatment effectively mimicked antigenic loss observed with human solid tumors. In addition to enhancing CAR T cell numbers, Super2 and IL-33 expression increased tumor infiltration of endogenous melanoma antigen-specific T cells. Generation of a broad antitumor response is essential for the field of CAR T cell therapy to effectively manage solid tumors. To date, there are limited tumor-specific antigens expressed in solid tumors, and those that are tumor selective are rarely expressed uniformly throughout the entire tumor. Thus, the expression of Super2 and IL-33 in CAR T cells has the potential to address antigen loss by activating innate cytotoxic NK and myeloid cells and priming endogenous T cells. It remains to be determined whether activated endogenous effector T cells can differentiate into long-term memory cells capable of the durable antitumor immunity.

IL-33 is an IL-1 family cytokine with alarmin properties that impact both innate and adaptive immune cells including, but not limited to macrophages, DCs, NK cells, T cells, and ILCs. Although IL-33 is a type 2 cytokine known for its ability to aid in type 2 immunity, its full activity remains unclear. Based on its activity in pancreatic cancer, we anticipated a role for IL-33 in activating, expanding, and mobilizing ILC2 cells, which then recruit CD103+ DCs to license and expand CD8s. However, the normal anti-tumor responses we observed with CD8- and RORα-deficient mice, which lack CD8 T cells and ILC2 cells, respectively led us to consider a broader role for IL-33. It has been reported that IL-33 can skew Th1 and Th2 T cell responses depending on the environmental conditions. Additionally, IL-33 has been shown to activate both macrophages and DC. Moreover, NK and NKT cells have been shown to respond to IL-33 by increasing IFNγ production and release upon receptor engagement. The modest reduction in tumor protection observed following administration of IFNγ-deficient CAR T cells supports further investigation of this mechanism downstream of IL-33. However, single cell RNA sequencing analysis supports a broad effect of IL-33 and Super2 inducing alterations to both T cell and macrophage responses. Future studies of IL-33-dependent mechanisms will assist in better understanding its role in anti-tumor responses.

In the clinic, systemically delivered high dose IL-2 can effectively activate the immune system and drive tumor clearance. While it has effected cures in a small subset of metastatic melanoma patients, toxicity remains a general concern, and coupled with a lack of predictive markers to identify responsive patients, it is not typically used as a single agent therapy today.

However, it is common practice for CART cells to be administered with systemic IL-2 to boost CAR T cell expansion, which often correlates with improved patient responsiveness and outcomes, but the toxicity concerns associated with high dose systemic IL-2 remain. By engineering CAR T cells to express IL-2, systemic toxicity may be ameliorated by direct delivery of IL-2 to tumors by CAR T cells, which utilize the CXCR3-CXCL9/10 axis to infiltrate solid tumors. While toxicity was not observed in our mouse tumor models, the more limited toxicity observed in mice compared to humans in response to systemic IL-2 suggests that careful pharmacodynamic studies need to be conducted unless additional safety measures, such as engineering kill switches or inducible IL-2 expression are used to de-risk the use of IL-2 and its variants.

The synergy that results from combining two diverse cytokines, Super2 and IL-33, is unexpectedly robust. Other type 2 cytokines were unable to synergize with Super2 except IL-25, which demonstrated partial efficacy. The partial overlap between IL-25 and IL-33 could perhaps be attributed to their shared ability to activate TRAF6 signaling, albeit downstream of distinct receptor associated adapter proteins41. In contrast, Super2, TSLP, IL-4 and IL-5 activate JAK/STAT signaling. Whether or not Super2 and IL-33 synergy occurs by activating JAK and TRAF6 pathways in cis, within the same cellular target or in trans within distinct cells remains to be determined. The ability of other type 1 cytokines such as IL-15 and IL-21 to synergize with IL-33 also may be demonstrated.

The high efficacy that Super2 and IL-33 CAR T cells exhibited in four tumor models suggests that Super2 and IL-33-based cell therapies can universally promote clearance of solid tumors independent of tumor type and tumor antigen. Additionally, the ability of the Super2 and IL-33 combination to induce antitumor responses in the absence of CAR expression offer hope that it could be applied to allogeneic therapies, which drastically reduce manufacturing time and costs and can be used off-the-shelf. It is also important to note that the anti-tumor responses observed occurred in the absence of pre-conditioning or combinations with other cancer treatments. We predict that combination with checkpoint blockade, which has dramatically impacted the cancer field due to its universality and efficacy will further increase antitumor immunity. This prediction is supported by our single cell RNA sequencing analysis, which identified increased portions of PD-1+ effector T cells in Super2 IL-33 CAR T cell treated TILs.

Based on the foregoing combining Super2 and IL-33 CAR T cells with checkpoint blockade immunotherapy has the potential to revitalize newly primed but exhausted endogenous CD8 T cells to drive complete tumor remission.

Although various embodiments and examples of the present invention have been described referring to certain molecules, compositions, methods, or protocols, it is to be understood that the present invention is not limited to the particular molecules, compositions, methods, or protocols described herein, as theses may vary. It is also to be understood that the terminology used in the description is for the purpose of describing the particular versions or embodiments only and is not intended to limit the scope of the present invention which will be limited only by the appended claims.

All references cited herein, including patent documents and non-patent documents, are hereby incorporated by reference in their entirety. Nothing herein is to be construed as an admission that the present invention is not entitled to antedate such disclosure by virtue of prior invention.

What is claimed is:

1. A method of treating cancer or infection, the method comprising administering to a subject in need thereof an immunostimulatory cytokine combination comprising a prophylactically or therapeutically effective amount of
(a) at least one Type I cytokine which comprises an interleukin-21 or interleukin-2-like (IL-2-like) cytokine, and
(b) at least one Type II cytokine, optionally IL-33 or IL-25, wherein said at least one cytokine (a) and (b) are administered in the same or different compositions, and preferably at dosages wherein said at least one cytokine (a) and (b) in combination elicit a synergistic or additive effect on immunity, e.g., antitumor or anti-infective immunity, compared to the administration of cytokine (a) or cytokine (b) alone.

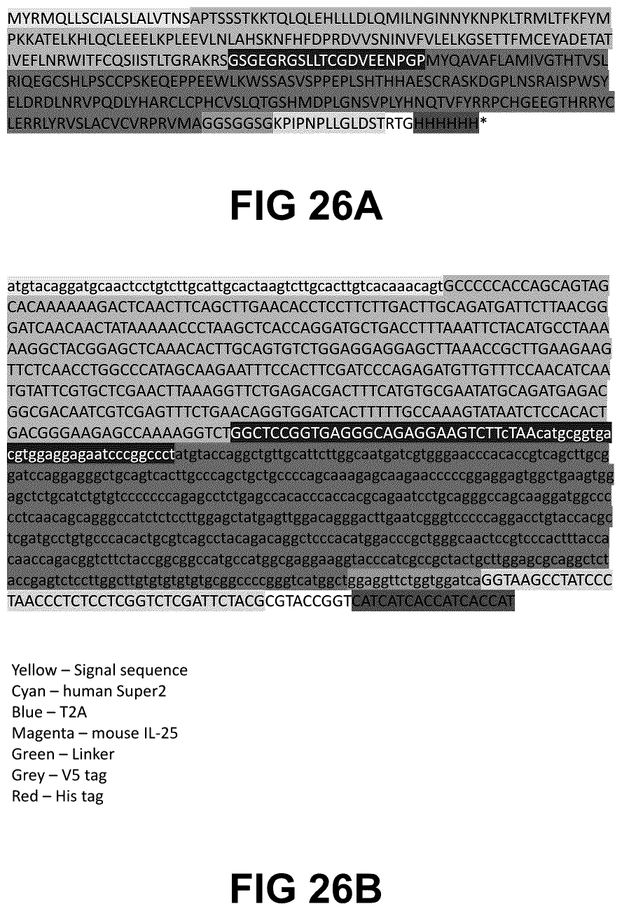

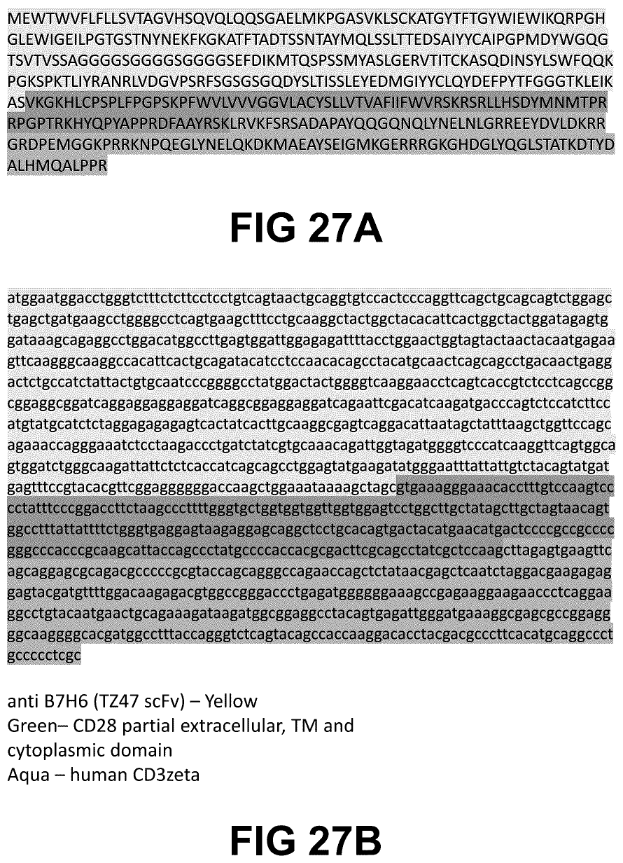

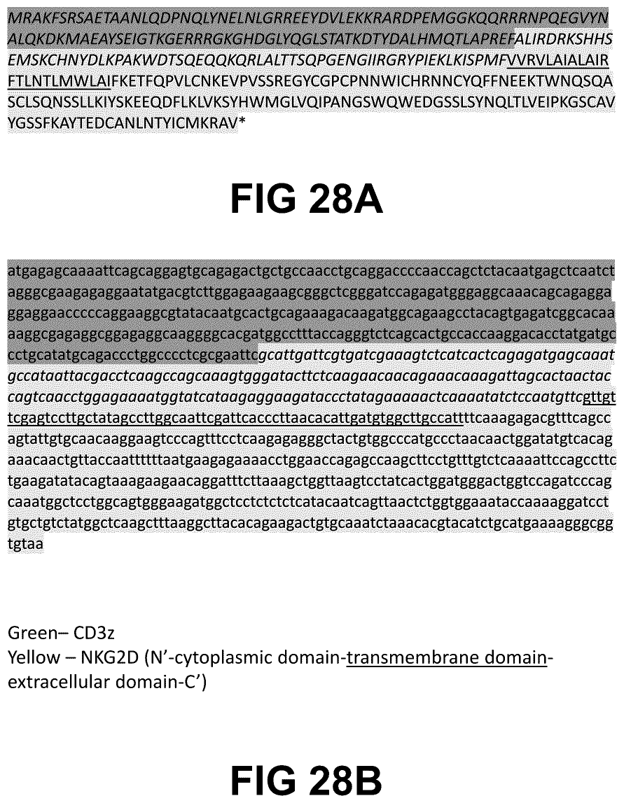

2. The method of claim 1 wherein
(i) the Type 1 cytokine is selected from IL-21, IL-2 (IL2), desleukin (Proleukin), Interking (recombinant IL-2 with a serine at residue 125), Neoleukin 2/15, IL2 fused to serum albumin (e.g., MSA/IL-2), or Super-2 (Super2) and other IL-2 variants and the Type 2 cytokine is selected from TSLP, IL-25 (IL25), IL-33, IL-1(IL1), IL-4 (IL4), IL-5 (IL5), IL-6 (IL6), IL-10 (IL10)4 (IL4), and IL-13 (IL13);
(ii) the immunostimulatory combination comprises Superkine IL-2 (Super-2) and IL-33, optionally wherein the Super-2 (i) has the amino acid sequence in FIG. 26A or it comprises a modified human IL-2 comprising one or more amino acid substitutions wherein numbering is relative to the native or endogenous human IL-2 polypeptide that increase IL-2RR binding affinity, further optionally wherein (ii) the one or more amino acid substitutions that increase IL-2Rβ binding affinity comprise or consist of substitutions selected from the group consisting of: Q74N, Q74H, Q74S, L80F, L8V, R81D, R81T, L85V, I86V, I89V, and I93V; or (iii) the amino acid substitutions comprise or consist of L80F, R81D, L85V, I86V, and I92F; or (iv) the amino acid substitutions comprise or consist of Q74N, L80F, R81D, L85V, I86V, I89V, and I92F; or (v) the amino acid substitutions comprise or consist of Q74N, L80V, R81T, L85V, I86V, and I92F; or (vi) the amino acid substitutions comprise or consist of Q74H, L80F, R81D, L85V, I86V, and I92F; or the amino acid substitutions comprise or consist of Q74S, L80F, R81D, L85V, I86V, and I92F; or (viii) the amino acid substitutions comprise or consist of Q74N, L80F, R81D, L85V, I86V, and I92F;
(iii) the immunostimulatory combination comprises Superkine IL-2 (Super-2) and IL-25 (IL25), optionally wherein the Super-2 has the amino acid sequence in FIG. 26A or it comprises one or more amino acid substitutions that increase IL-2Rβ binding affinity, wherein (ii) the one or more amino acid substitutions that increase IL-2Rβ binding affinity comprise or consist of substitutions selected from the group consisting of: Q74N, Q74H, Q74S, L80F, L8V, R81D, R81T, L85V, I86V, I89V, and I93V; or (iii) the amino acid substitutions comprise or consist of L80F, R81D, L85V, I86V, and I92F; or (iv) the amino acid substitutions comprise or consist of Q74N, L80F, R81D, L85V, I86V, I89V, and I92F; or (v) the amino acid substitutions comprise or consist of Q74N, L80V, R81T, L85V, I86V, and I92F; or (vi) the amino acid substitutions comprise or consist of Q74H, L80F, R81D, L85V, I86V, and I92F; or the amino acid substitutions comprise or consist of Q74S, L80F, R81D, L85V, I86V, and I92F; or (viii) the amino acid substitutions comprise or consist of Q74N, L80F, R81D, L85V, I86V, and I92F;
(iv) is used to treat cancer;
(v) is used to treat a solid tumor;
(vi) is used to treat a solid tumor, wherein the solid tumor is selected from bladder cancer, bone cancer, brain cancer, breast cancer, carcinomas, cervical cancer, colon cancer, colorectal cancer, desmoid tumor, endometrial cancer, esophageal cancer, fibromatosis, glioblastoma, head and neck cancer, liver cancer, lung cancer, lymphoma, leukemias, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, testicular cancer, thyroid cancer, urothelial cancer, or a combination of any of the foregoing;
(vii) said immunostimulatory combination additively or synergistically activates the endogenous immune response providing for enhanced inhibition of tumor growth and/or metastasis;
(viii) either or both of said cytokines are administered in soluble form, optionally wherein either or both of said cytokines are modified to enhance in vivo half-life, optionally by lipidation, conjugation to one or more polyethylene glycols, albumins such as human serum albumin, Fc polypeptides, XTEN protein polymers, or multimerization;
(ix) either or both of said cytokines are expressed by a recombinant cell, optionally an immune cell;
(x) either or both of said cytokines are expressed by a recombinant cell, optionally a cell which expresses an endogenous or chimeric receptor or binding domain, further optionally an antibody binding domain, which binds to a ligand or receptor expressed by target cells, e.g., cancer or infected cells;
(xi) either or both of said cytokines are expressed by a CAR expressing cell, e.g., a CAR-T or CAR-NK cell;
(xii) the method includes administering cells which express either or both of Super-2 and IL-33 and further express a receptor or binding domain that binds to an antigen or ligand expressed on target cells, e.g., a natural or synthetic or chimeric receptor or an antibody or antibody fragment that binds to target (e.g., tumor) cells;
(xiii) the method includes administering cells which express either or both of Super-2 and IL-25 and further express a receptor or binding domain that binds to an antigen or ligand expressed on target cells, optionally a natural or synthetic or chimeric receptor or an antibody or antibody fragment that binds to target, optionally tumor cells;
(xiv) said at least one Type I and Type II cytokines are administered separately;
(xv) said at least one Type I and Type II cytokines are administered in combination;
(xvi) either or both of the Type I and Type II cytokines are administered as a soluble protein, optionally via injection, further optionally by intraperitoneal administration, intravenous infusion, intramuscularly, intratumorally, subcutaneously; sublingually, intranasally, or other conventional route of administration of soluble proteins;
(xvii) said at least one Type I and Type II cytokine in combination elicit a synergistic effect on immunity, optionally on antitumor immunity;
(xviii) either or both of said Type I and Type II cytokines are expressed by a recombinant or isolated cell which expresses either or both of said Type I and Type 11 cytokines under constitutive or inducible conditions, optionally wherein said recombinant or isolated cell optionally comprises an immune cell, further optionally selected from the group consisting of a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH17 cell, a TH9 cell, a TH22 cell, a follicular helper T cells, an a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, a dendritic cell, and an interferon gamma-deficient cell, preferably a CAR-T or CAR-NK cell; and/or wherein the recombinant or isolated cell expresses at least one natural or chimeric antigen receptor (CAR) which binds to an antigen expressed by target cells, optionally tumor, infected or other diseased cells;
(xix) the at least one chimeric antigen receptor (CAR) comprises:
(a) an antigen-binding (AB) domain that binds to a target molecule which is expressed on the surface of a tumor, a virus, or virus infected cell in a patient or which is overexpressed or aberrantly expressed in cancer, viral infection, or other disease associated with expression of the antigen bound by the AB domain,
(b) a transmembrane (TM) domain,
(c) an intracellular signaling (ICS) domain,
(d) optionally a hinge that joins said AB domain and said TM domain, and
(e) optionally one or more additional costimulatory (CS) domains;
(xx) the administered recombinant or isolated cell of (xviii) or (xix) comprises at least one nucleic acid encoding a CAR and at least one nucleic acid encoding said Type I cytokine and/or said Type 11 cytokine;
(xxi) the administered recombinant or isolated cell of (xviii), (xix) or (xx) is (1) activated or stimulated to proliferate when the CAR binds to its target molecule; (2) the cell is activated or stimulated to release or secrete said type 1 and/or said type II cytokine when the CAR binds to its target molecule; (3) the cell exhibits cytotoxicity against cells expressing the target molecule when the CAR binds to the target molecule; (4) administration of the cell ameliorates a disease, e.g., an infectious disease or a neoplastic disease, e.g., a solid tumor associated condition, when the CAR binds to its target molecule; (5) administration of the cell alone or in combination with a type I and/or type II cytokine shifts the disease microenvironment from immune suppressive to immune stimulatory; (6) administration of the cell alone or in combination with a type I and/or type II cytokine increases populations of infiltrating immune cells within the disease microenvironment; and/or (7) administration of the cell alone or in combination with a type I and/or type II cytokine results in enhanced killing of target cells because of the additive or synergistic effects of the combination of the type I and type II cytokine on immunity, optionally antitumor immunity;
(xxii) the method of any one of (i) to (xxi), wherein,
(a) both Type I and Type 11 cytokines are administered as soluble proteins,
(b) both Type I and Type 11 cytokines are expressed by a recombinant or isolated cell, e.g., a CAR-T cell engineered to express said Type I and Type 11 cytokines, or
(c) either the type I cytokine or the type II cytokine is administered as a soluble protein and the other cytokine is expressed by a recombinant or isolated cell, e.g., a CAR-T cell engineered to express said Type I or Type 11 cytokine;
(xxiii) is used for stimulating an immune response in a subject in need thereof, optionally a synergistic immune response, further optionally a synergistic antitumor immune response, optionally against a solid tumor;
(xxiv) is used for stimulating an immune response in a subject in need thereof, optionally a synergistic immune response, further optionally a synergistic antitumor immune response, preferably against a solid tumor is stimulated in subject in need thereof which is immunoreplete and is similarly stimulated in a subject in need thereof which lacks sufficient endogenous response from immune cells selected from the group comprising CD8, CD4, NK, and/or ILC2 cells;
(xxvi) is used is used to treat cancer, wherein the cancer is selected from the group consisting of bladder cancer, bone cancer, brain cancer, breast cancer, carcinomas, cervical cancer, colon cancer, colorectal cancer, desmoid tumor, endometrial cancer, esophageal cancer, fibromatosis, glioblastoma, head and neck cancer, liver cancer, lung cancer, lymphoma, leukemias, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, testicular cancer, thyroid cancer, urothelial cancer, or a combination of any of the foregoing; or
(xxvii) is used is used in the treatment of infectious disease, wherein the infection is selected from the group consisting of viral, bacterial, yeast, fungal, or parasite.

3-29. (canceled)

30. A composition comprising an immunostimulatory combination comprising at least one immune stimulating Type I cytokine, optionally selected from the group consisting of interleukin-21, interleukin-2 (IL-2), and an IL-2 superkine variant (super-2) optionally wherein said super-2 has the amino acid sequence shown in FIG. 26A or it comprises one or more amino acid substitutions wherein numbering of the substitutions is relative to the native or endogenous human IL-2 polypeptide that increase IL-2Rβ binding affinity, optionally wherein (ii) the one or more amino acid substitutions that increase IL-2Rβ binding affinity comprise or consist of substitutions selected from the group consisting of: Q74N, Q74H, Q74S, L80F, L8V, R81D, R81T, L85V, I86V, I89V, and I93V; or (iii) the amino acid substitutions comprise or consist of L80F, R81D, L85V, I86V, and I92F; or (iv) the amino acid substitutions comprise or consist of Q74N, L80F, R81D, L85V, I86V, I89V, and I92F; or (v) the amino acid substitutions comprise or consist of Q74N, L80V, R81T, L85V, I86V, and I92F; or (vi) the amino acid substitutions comprise or consist of Q74H, L80F, R81D, L85V, I86V, and I92F; or the amino acid substitutions comprise or consist of Q74S, L80F, R81D, L85V, I86V, and I92F; or (viii) the amino acid substitutions comprise or consist of Q74N, L80F, R81D, L85V, I86V, and I92F; preferably super-2 and a Type II cytokine optionally selected from the group consisting of interleukin-4 (IL-4), interleukin-5 (IL-5), interleukin-25 (IL-25), interleukin-33 (IL-33), and Thymic stromal lymphopoietin (TSLP), or an immunologically active fragment, variant or fusion protein comprising any of the foregoing, preferably IL-33, an IL-33 fusion protein, IL-25, or an IL-25 fusion protein which Type I and Type II cytokine when administered in combination elicits an additive or synergistic effect on immunity; wherein optionally:
(i) the Type I cytokine comprises or consists of an amino acid sequence at least 80%, at least 85%, at least 90%, at least 92%, at least 94%, at least 95%, at least 96%, at least 97%, at least 98%, at least 99%, or 100% identical to human IL-21, IL-2 or Super-2 immunologically active fragments, variants and fusion proteins comprising any of the foregoing;
(ii) the Type II cytokine is selected from the group consisting of interleukin-4 (IL-4), interleukin-5 (IL-5), interleukin-25 (IL-25), interleukin-33 (IL-33), and Thymic stromal lymphopoietin (TSLP) and immunologically active fragments, variants and fusion proteins comprising any of the foregoing; or
(iii) the Type II cytokine has an amino acid sequence at least 80%, at least 85%, at least 90%, at least 92%, at least 94%, at least 95%, at least 96%, at least 97%, at least 98%, at least 99%, or 100% identical to endogenous IL-4, IL-5, IL-25, IL-33, TSLP, or another Type II cytokine, preferably IL-33 or IL-25.

31-33. (canceled)

34. An isolated or recombinant cell which is engineered to express at least one Type I cytokine which comprises an IL-21 or IL-2 like cytokine, optionally selected from the group consisting of interleukin-2 (IL-2), further optionally human IL-2, an IL-2 superkine variant (super-2) or other IL-2 variant; and at least one Type II cytokine, optionally selected from the group consisting of interleukin-4 (IL-4), interleukin-5 (IL-5), interleukin-25 (IL-25), interleukin-33 (IL-33), and Thymic stromal lymphopoietin (TSLP), preferably IL-33, an IL-33 fusion protein, IL-25, or an IL-25 fusion protein which cytokines when expressed in combination elicit an additive or synergistic effect on immunity, optionally antitumor immunity; which optionally:
(i) comprises an immune cell, optionally selected from the group consisting of a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH17 cell, a TH9 cell, a TH22 cell, a follicular helper T cells, an a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, an interferon gamma-deficient cell, a B cell, a myeloid cell, a monocyte, a macrophage, and a dendritic cell;
(ii) further expresses a chimeric antigen receptor (CAR) comprising:
(a) an antigen-binding (AB) domain that binds to a target molecule which is expressed on the surface of a tumor, a virus, or virus infected cell or which is overexpressed or aberrantly expressed in cancer or viral infection, or other disease,
(b) a transmembrane (TM) domain,
(c) an intracellular signaling (ICS) domain,
(d) optionally a hinge that joins said AB domain and said TM domain, and
(e) optionally one or more costimulatory (CS) domains, which CAR optionally binds to a target molecule selected from an antigen, ligand or receptor expressed by tumor or infected cells, optionally a Tyrosinase-related protein 1 (TYRP1), a stress-inducible natural killer (NK) cell lig and (B7H6 et al.), or an induced-self antigen from the MIC and/or RAET1/ULBP families which are inducible by stress, malignant transformation, or infection (Rael et al.); and/or optionally the AB domain in the CAR comprises an antibody (Ab) or an antigen-binding fragment thereof that binds to said target molecule, wherein said Ab or antigen-binding fragment thereof is optionally selected from a group consisting of a monoclonal Ab, a monospecific Ab, a polyspecific Ab, a humanized Ab, a tetrameric Ab, a tetravalent Ab, a multispecific Ab, a single chain Ab, a domain-specific Ab, a single-domain Ab (dAb), a domain-deleted Ab, an scFc fusion protein, a chimeric Ab, a synthetic Ab, a recombinant Ab, a hybrid Ab, a mutated Ab, CDR-grafted Ab, a fragment antigen-binding (Fab), an F(ab′)2, an Fab′ fragment, a variable fragment (Fv), a single-chain Fv (scFv) fragment, an Fd fragment, a dAb fragment, a diabody, a nanobody, a bivalent nanobody, a shark variable IgNAR domain, a V_{HH} Ab, a camelid Ab, and a minibody; and/or optionally the TM domain in the CAR is derived from the TM region, or a membrane-spanning portion thereof, of a protein selected from the group consisting of CD28, CD3e, CD4, CD5, CD8, CD8α, CD9, CD16, CD22, CD33, CD37, CD45, CD64, CD80, CD86, CD134, CD137, CD154, TCRa, TCRb, and CD3z; and/or optionally the CAR comprises a TM domain is derived from the TM region of CD28, or a membrane-spanning portion thereof; and/or optionally the CAR comprises an ICS domain derived from a cytoplasmic signaling sequence, or a functional fragment thereof, of a protein selected from the group consisting of CD3, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD28, CD66d, CD79a, CD79b, CD278 (ICOS), FceRI, 4-1BB, DAP10, and DAP12; and/or optionally the CAR comprises an ICS domain derived from a cytoplasmic signaling sequence of CD3, or a functional fragment thereof; and/or optionally the CAR comprises a hinge derived from CD28 or other costimulatory protein; and/or optionally the CAR comprises one or more CS domains if present is/are derived from a cytoplasmic signaling sequence, or functional fragment thereof, of a protein selected from the group consisting of CD28, DAP10, 4-1BB (CD137), CD2, CD4, CD5, CD7, CD8α, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAMI, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and CD83 ligand.

35-44. (canceled)

45. A nucleic acid construct which comprises a nucleic acid that encodes for (1) a type I cytokine, optionally selected from the group consisting of interleukin-21, interleukin-2 (IL-2), and (2) an IL-2 superkine variant (super-2); preferably super-2, optionally wherein said encoded super-2 has the amino acid sequence of the super-2 shown in FIG. 26A or the encoded super-2 comprises one or more amino acid substitutions in the native or endogenous human IL-2 sequence wherein numbering of the substitutions is relative to the native or endogenous human IL-2 polypeptide which substitutions increase IL-2Rβ binding affinity, wherein optionally (ii) the one or more amino acid substitutions that increase IL-2Rβ binding affinity comprise or consist of substitutions selected from the group consisting of: Q74N, Q74H, Q74S, L80F, L8V, R81D, R81T, L85V, I86V, I89V, and I93V; or (iii) the amino acid substitutions comprise or consist of L80F, R81D, L85V, I86V, and I92F; or (iv) the amino acid substitutions comprise or consist of Q74N, L80F, R81D, L85V, I86V, I89V, and I92F; or (v) the amino acid substitutions comprise or consist of Q74N, L80V, R81T, L85V, I86V, and I92F; or (vi) the amino acid substitutions comprise or consist of Q74H, L80F, R81D, L85V, I86V, and I92F; or the amino acid substitutions comprise or consist of Q74S, L80F, R81D, L85V, I86V, and I92F; or (viii) the amino acid substitutions comprise or consist of Q74N, L80F, R81D, L85V, I86V, and I92F, and a nucleic acid that encodes for a type II cytokine, optionally selected from the group consisting of interleukin-4 (IL-4), interleukin-5 (IL-5), interleukin-25 (IL-25), interleukin-33 (IL-33), and Thymic stromal lymphopoietin (TSLP), preferably IL-33 or IL-25, which Type I and Type II cytokine when expressed in combination elicit an additive or synergistic effect on immunity; which optionally
(i) further encodes a natural receptor or a CAR which binds to target cells, wherein the CAR optionally comprises
(a) an AB domain or receptor that binds to a ligand, antigen or receptor expressed by target cells, e.g., tumor or infected cells,
(b) a transmembrane (TM) domain,
(c) an intracellular signaling (ICS) domain,
(d) optionally a hinge that joins said AB domain and said TM domain, and
(e) optionally one or more costimulatory (CS) domains;
(ii) further encodes an Ab or Ab fragment, wherein the Ab or Ab fragment binds to an antigen expressed on tumor cells, e.g., bladder cancer, bone cancer, brain cancer, breast cancer, carcinomas, cervical cancer, colon cancer, colorectal cancer, desmoid tumor, endometrial cancer, esophageal cancer, fibromatosis, glioblastoma, head and neck cancer, liver cancer, lung cancer, lymphoma, leukemias, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, testicular cancer, thyroid cancer, urothelial cancer, or a combination of any of the foregoing;
(iii) further encodes a CAR wherein the CAR comprises a TM domain derived from the TM region, or a membrane-spanning portion thereof, of a protein selected from the group consisting of CD28, CD3e, CD4, CD5, CD8, CD8α, CD9, CD16, CD22, CD33, CD37, CD45, CD64, CD80, CD86, CD134, CD137, CD154, TCRa, TCRb, and CD3z;
(iv) further encodes a CAR wherein the CAR comprises a TM domain derived from the TM region of CD28, or a membrane-spanning portion thereof;
(v) further encodes a CAR wherein the CAR comprises a ICS domain derived from a cytoplasmic signaling sequence, or a functional fragment thereof, of a protein selected from the group consisting of CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD28, CD66d, CD79a, CD79b, CD278 (ICOS), FceRI, 4-1BB, DAP10, and DAP12; or
(vi) further encodes a CAR wherein the CAR comprises at least one or more CS domains is derived from a cytoplasmic signaling sequence, or functional fragment thereof, of a protein selected from the group consisting of CD28, DAP10, 4-1BB (CD137), CD2, CD4, CD5, CD7, CD8α, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAMI, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and CD83 ligand.

46-51. (canceled)

52. A vector comprising the nucleic acid of claim 45, wherein the vector is selected from a DNA, an RNA, a plasmid, a cosmid, a viral vector, a lentiviral vector, an adenoviral vector, or a retroviral vector.

53. (canceled)

54. A recombinant or isolated cell comprising a nucleic acid according to claim 45; optionally
(i) a non-mammalian cell, further optionally selected from the group consisting of a plant cell, a bacterial cell, a fungal cell, a yeast cell, a protozoa cell, and an insect cell, or
(ii) a mammalian cell, optionally selected from the group consisting of a human cell, a rat cell, and a mouse cell;
(iii) a stem cell;
(iv) a primary cell, optionally a human primary cell or derived therefrom;
(v) a cell line, optionally a hybridoma cell line;
(vi) an immune cell; is MHC+ or MHC−;
(vii) the cell is selected from the group consisting of a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH17 cell, a TH9 cell, a TH22 cell, a follicular helper T cells, an a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, an interferon gamma-deficient cell, a B cell, a myeloid cell, a monocyte, a macrophage, and a dendritic cell;
(viii) is a T cell or T cell progenitor cell; (ix) the cell is a T cell which has been modified such that its endogenous T cell receptor (TCR) is
(a) not expressed,
(b) not functionally expressed, or
(c) expressed at reduced levels compared to a wild-type T cell;
(ix) the cell is activated or stimulated to proliferate and/or to release at least one cytokine, and/or to shift the disease microenvironment from immune suppressive to immune stimulatory, and/or to increase populations of infiltrating immune cells within the disease microenvironment, and/or to increase expression of cytokines and/or chemokines and/or to elicit killing of target cells when it binds to its target molecule; or
(x) administration of the cell ameliorates a disease, preferably cancer or infectious disease, optionally a solid tumor.

55-66. (canceled)

67. A population of cells comprising at least one recombinant or isolated cell according to claim 54.

68. A pharmaceutical composition comprising (a) a cytokine combination and/or at least one recombinant cell which expresses a cytokine combination according to claim 1 and (b) a pharmaceutically acceptable excipient or carrier.


SEQUENCE LISTING (12 sequences)

SEQ ID NO: 1 - DNA, 1078 bp, synthetic construct
  misc_feature 1..1078: note = Tailless TA99 CAR sequence
  source 1..1078: mol_type = other DNA
atggaggcccccgcccagctgctgttcctgctgctgctgtggctgcccagcagcagggccgaggttcagctccaacagtctggggctgagcttgtgaggccaggggccttggtcaagttgtcctgcaaaacttctggcttcaacattaaagactactttttacactgggtgagacagaggcctgaccagggcctggagtggattggatggattaatcctgataatggtaatactgtttatgacccgaagtttcagggcacggccagtttaacagcagacacatcctccaacacagtctacttgcagctcagcggcctgacatctgaggacactgccgtctatttctgtactcggagggactatacttatgaaaaggctgctctggactactggggtcagggagcctcagtcatcgtctcctcaggtggaggtggatcaggtggaggtggatctggtggaggtggatctgacatccagatgtctcagtctccagcctccctatctgcatctgtgggagaaactgtcaccatcacatgtcgagcaagtggaaatatttacaattatttagcatggtatcagcagaaacagggaaaatctcctcacctcctggtctatgatgcaaaaaccttagcagatggtgtgccatcaaggttcagtggcagtggctcagggacacaatattctctcaagattagcagcctgcagactgaagattctgggaattattactgtcaacatttttggagtcttccattcacgttcggctcggggacaaagttggaaataaaacgggctggtggtggttctggtggtggtgaacagaagctgatcagcgaggaggatctgactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatcaatagtagaaggaacagactccttcaaagtgact

SEQ ID NO: 2 - protein, 359 aa, synthetic construct
  REGION 1..359: note = Tailless TA99 CAR sequence
MEAPAQLLFLLLLWLPSSRAEVQLQQSGAELVRPGALVKLSCKTSGFNIKDYFLHWVRQRPDQGLEWIGWINPDNGNTVYDPKFQGTASLTADTSSNTVYLQLSGLTSEDTAVYFCTRRDYTYEKAALDYWGQGASVIVSSGGGGSGGGGSGGGGSDIQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIKRAGGGSGGGEQKLISEEDLTTTKPVLRTPSPVHPTGTSQPQRPEDCRPRGSVKGTGLDFACDIYIWAPLAGICVALLLSLIITLINSRRNRLLQSD

SEQ ID NO: 3 - protein, 502 aa, synthetic construct
  REGION 1..502: note = anti-TYRP1 CAR sequence
MEAPAQLLFLLLLWLPSSRAEVQLQQSGAELVRPGALVKLSCKTSGFNIKDYFLHWVRQRPDQGLEWIGWINPDNGNTVYDPKFQGTASLTADTSSNTVYLQLSGLTSEDTAVYFCTRRDYTYEKAALDYWGQGASVIVSSGGGGSGGGGSGGGGSDIQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIKRAGGGSGGGEQKLISEEDLTTTKPVLRTPSPVHPTGTSQPQRPEDCRPRGSVKGTGLDFACDIYIWAPLAGICVALLLSLIITLINSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 4 - DNA, 1509 bp, synthetic construct
  misc_feature 1..1509: note = schematic of a CAR sequence
  source 1..1509: mol_type = other DNA
atggaggcccccgcccagctgctgttcctgctgctgctgtggctgcccagcagcagggccgaggttcagctccaacagtctggggctgagcttgtgaggccaggggccttggtcaagttgtcctgcaaaacttctggcttcaacattaaagactactttttacactgggtgagacagaggcctgaccagggcctggagtggattggatggattaatcctgataatggtaatactgtttatgacccgaagtttcagggcacggccagtttaacagcagacacatcctccaacacagtctacttgcagctcagcggcctgacatctgaggacactgccgtctatttctgtactcggagggactatacttatgaaaaggctgctctggactactggggtcagggagcctcagtcatcgtctcctcaggtggaggtggatcaggtggaggtggatctggtggaggtggatctgacatccagatgtctcagtctccagcctccctatctgcatctgtgggagaaactgtcaccatcacatgtcgagcaagtggaaatatttacaattatttagcatggtatcagcagaaacagggaaaatctcctcacctcctggtctatgatgcaaaaaccttagcagatggtgtgccatcaaggttcagtggcagtggctcagggacacaatattctctcaagattagcagcctgcagactgaagattctgggaattattactgtcaacatttttggagtcttccattcacgttcggctcggggacaaagttggaaataaaacgggctggtggtggttctggtggtggtgaacagaagctgatcagcgaggaggatctgactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatcaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggacccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctga

SEQ ID NO: 5 - protein, 392 aa, synthetic construct
  REGION 1..392: note = cytokine construct
MYRMQLLSCIALSLALVTNSAPTSSSTKKTQLQLEHLLLDLQMILNGINNYKNPKLTRMLTFKFYMPKKATELKHLQCLEEELKPLEEVLNLAHSKNFHFDPRDVVSNINVFVLELKGSETTFMCEYADETATIVEFLNRWITFCQSIISTLTGRAKRSGSGEGRGSLITCGDVEENPGPTIGAGKQEQQTDKSDPFHAASVDTLSIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKIGGSGGSGKPIPNPLLGLDSTRTGHHHHHH

SEQ ID NO: 6 - DNA, 1176 bp, synthetic construct
  misc_feature 1..1176: note = schematic of a cytokine sequence
  source 1..1176: mol_type = other DNA
atgtacaggatgcaactcctgtcttgcattgcactaagtcttgcacttgtcacaaacagtgcccccaccagcagtagcacaaaaaagactcaacttcagcttgaacacctccttcttgacttgcagatgattcttaacgggatcaacaactataaaaaccctaagctcaccaggatgctgacctttaaattctacatgcctaaaaaggctacggagctcaaacacttgcagtgtctggaggaggagcttaaaccgcttgaagaagttctcaacctggcccatagcaagaatttccacttcgatcccagagatgttgtttccaacatcaatgtattcgtgctcgaacttaaaggttctgagacgactttcatgtgcgaatatgcagatgagacggcgacaatcgtcgagtttctgaacaggtggatcactttttgccaaagtataatctccacactgacgggaagagccaaaaggtctggctccggtgagggcagaggaagtcttataacatgcggtgacgtggaggagaatcccggccctactataggcgctggaaaacaagaacaacaaactgataaaagtgacccatttcatgctgcgtctgttgacacattgagcatccaaggaacttcacttttaacacagtctcctgcctccctgagtacatacaatgaccaatctgttagttttgttttggagaatggatgttatgtgatcaatgttgacgactctggaaaagaccaagagcaagaccaggtgctactacgctactatgagtctccctgtcctgcaagtcaatcaggcgacggtgtggatgggaagaagctgatggtgaacatgagtcccatcaaagacacagacatctggctgcatgccaacgacaaggactactccgtggagcttcaaaggggtgacgtctcgcctccggaacaggccttcttcgtccttcacaaaaagtcctcggactttgtttcatttgaatgcaagaatcttcctggcacttacataggagtaaaagataaccagctggctctagtggaggagaaagatgagagctgcaacaatattatgtttaagctctcgaaaatcggaggttctggtggatcaggtaagcctatccctaaccctctcctcggtctcgattctacgcgtaccggtcatcatcaccatcaccat

SEQ ID NO: 7 - protein, 378 aa, synthetic construct
  REGION 1..378: note = immunostimulatory cytokine construct comprising Super2 and IL25
MYRMQLLSCIALSLALVTNSAPTSSSTKKTQLQLEHLLLDLQMILNGINNYKNPKLTRMLTFKFYMPKKATELKHLQCLEEELKPLEEVLNLAHSKNFHFDPRDVVSNINVFVLELKGSETTFMCEYADETATIVEFLNRWITFCQSIISTLTGRAKRSGSGEGRGSLLTCGDVEENPGPMYQAVAFLAMIVGTHTVSLRIQEGCSHLPSCCPSKEQEPPEEWLKWSSASVSPPEPLSHTHHAESCRASKDGPLNSRAISPWSYELDRDLNRVPQDLYHARCLCPHCVSLQTGSHMDPLGNSVPLYHNQTVFYRRPCHGEEGTHRRYCLERRLYRVSLACVCVRPRVMAGGSGGSGKPIPNPLLGLDSTRTGHHHHHH

SEQ ID NO: 8 - DNA, 1134 bp, synthetic construct
  misc_feature 1..1134: note = immunostimulatory cytokine construct comprising Super2 and IL25
  source 1..1134: mol_type = other DNA
atgtacaggatgcaactcctgtcttgcattgcactaagtcttgcacttgtcacaaacagtgcccccaccagcagtagcacaaaaaagactcaacttcagcttgaacacctccttcttgacttgcagatgattcttaacgggatcaacaactataaaaaccctaagctcaccaggatgctgacctttaaattctacatgcctaaaaaggctacggagctcaaacacttgcagtgtctggaggaggagcttaaaccgcttgaagaagttctcaacctggcccatagcaagaatttccacttcgatcccagagatgttgtttccaacatcaatgtattcgtgctcgaacttaaaggttctgagacgactttcatgtgcgaatatgcagatgagacggcgacaatcgtcgagtttctgaacaggtggatcactttttgccaaagtataatctccacactgacgggaagagccaaaaggtctggctccggtgagggcagaggaagtcttctaacatgcggtgacgtggaggagaatcccggccctatgtaccaggctgttgcattcttggcaatgatcgtgggaacccacaccgtcagcttgcggatccaggagggctgcagtcacttgcccagctgctgccccagcaaagagcaagaacccccggaggagtggctgaagtggagctctgcatctgtgtcccccccagagcctctgagccacacccaccacgcagaatcctgcagggccagcaaggatggccccctcaacagcagggccatctctccttggagctatgagttggacagggacttgaatcgggtcccccaggacctgtaccacgctcgatgcctgtgcccacactgcgtcagcctacagacaggctcccacatggacccgctgggcaactccgtcccactttaccacaaccagacggtcttctaccggcggccatgccatggcgaggaaggtacccatcgccgctactgcttggagcgcaggctctaccgagtctccttggcttgtgtgtgtgtgcggccccgggtcatggctggaggttctggtggatcaggtaagcctatccctaaccctctcctcggtctcgattctacgcgtaccggtcatcatcaccatcaccat

SEQ ID NO: 9 - protein, 461 aa, synthetic construct
  REGION 1..461: note = exemplary anti-B7H6 (TZ47) CAR construct
MEWTWVFLFLLSVTAGVHSQVQLQQSGAELMKPGASVKLSCKATGYTFTGYWIEWIKQRPGHGLEWIGEILPGTGSTNYNEKFKGKATFTADTSSNTAYMQLSSLTTEDSAIYYCAIPGPMDYWGQGTSVTVSSAGGGGSGGGGSGGGGSEFDIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIKASVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 1383 bp, synthetic construct
  misc_feature 1..1383: note = exemplary anti-B7H6 (TZ47) CAR construct
  source 1..1383: mol_type = other DNA
atggaatggacctgggtctttctcttcctcctgtcagtaactgcaggtgtccactcccaggttcagctgcagcagtctggagctgagctgatgaagcctggggcctcagtgaagctttcctgcaaggctactggctacacattcactggctactggatagagtggataaagcagaggcctggacatggccttgagtggattggagagattttacctggaactggtagtactaactacaatgagaagttcaagggcaaggccacattcactgcagatacatcctccaacacagcctacatgcaactcagcagcctgacaactgaggactctgccatctattactgtgcaatcccggggcctatggactactggggtcaaggaacctcagtcaccgtctcctcagccggcggaggcggatcaggaggaggaggatcaggcggaggaggatcagaattcgacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgtacacgttcggaggggggaccaagctggaaataaaagctagcgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 11 - protein, 347 aa, synthetic construct
  REGION 1..347: note = exemplary anti-Rae1 CAR construct comprising mouse NKG2D full length type 2 membrane protein
MRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPREFALIRDRKSHHSEMSKCHNYDLKPAKWDTSQEQQKQRLALTTSQPGENGIIRGRYPIEKLKISPMFVVRVLAIALAIRFTLNTLMWLAIFKETFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 12 - DNA, 1044 bp, synthetic construct
  misc_feature 1..1044: note = exemplary anti-Rae1 CAR construct
  source 1..1044: mol_type = other DNA
atgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgcgaattcgcattgattcgtgatcgaaagtctcatcactcagagatgagcaaatgccataattacgacctcaagccagcaaagtgggatacttctcaagaacaacagaaacaaagattagcactaactaccagtcaacctggagaaaatggtatcataagaggaagataccctatagaaaaactcaaaatatctccaatgttcgttgttcgagtccttgctatagccttggcaattcgattcacccttaacacattgatgtggcttgccattttcaaagagacgtttcagccagtattgtgcaacaaggaagtcccagtttcctcaagagagggctactgtggcccatgccctaacaactggatatgtcacagaaacaactgttaccaattttttaatgaagagaaaacctggaaccagagccaagcttcctgtttgtctcaaaattccagccttctgaagatatacagtaaagaagaacaggatttcttaaagctggttaagtcctatcactggatgggactggtccagatcccagcaaatggctcctggcagtgggaagatggctcctctctctcatacaatcagttaactctggtggaaataccaaaaggatcctgtgctgtctatggctcaagctttaaggcttacacagaagactgtgcaaatctaaacacgtacatctgcatgaaaagggcggtgtaa